(19) United States
(12) Patent Application Publication
(10) Pub. No.: US 2026/0021248 A1
(43) Pub. Date: Jan. 22, 2026

(54) SYSTEMS AND METHODS FOR ADMINISTRATION OF DRUGS

(71) Applicant: MEDPHLOW, LLC, Richmond, VA (US)

(72) Inventors: Eric S. EDWARDS, Moseley, VA (US); David JOHNSTON, Moseley, VA (US); John MCCURLEY, Powhatan, VA (US); Cristian DE LA COTERA, Minneapolis, MN (US); Siraaj DHRU, Minneapolis, MN (US); Dallas ERDAHL, Minneapolis, MN (US); Emma FAVILL, Minneapolis, MN (US); Dan JOHNSON, Minneapolis, MN (US); Greg JOHNSON, Minneapolis, MN (US); Jonathan SOLLIE, Minneapolis, MN (US); Victor STIVALA, Bethlehem, PA (US); Brian JAMES, Newton, MA (US); Andrew KING, King of Prussia, PA (US); Stephen WATT, Charleston, SC (US)

(73) Assignee: MEDPHLOW, LLC, Richmond, VA (US)

(21) Appl. No.: 19/108,270

(22) PCT Filed: Sep. 7, 2023
(86) PCT No.: PCT/US2023/032183

§ 371 Date: Mar. 3, 2025

Related U.S. Application Data
(60) Provisional application No. 63/405,055, filed on Sep. 9, 2022.

(51) Int. Cl. A61M 5/142; A61M 5/168; A61M 5/172

(52) U.S. Cl. CPC A61M 5/1422; A61M 5/16827; A61M 5/1723

(57) ABSTRACT

An automated medication administration system that is employed with an integrated cartridge assembly in response to the acute medical condition. The pump assembly is configured to engage the cartridge assembly having at least a first container containing saline and a second container containing a medicament. The engagement of the cartridge assembly transitions a gate member of the cartridge assembly from a locked position to an unlocked position. The pump includes a drive assembly that is configured to convey repeated doses of the medicament in accordance with a treatment protocol when the gate member is in the unlocked position. Each dose of the medicament is followed by a dose of the saline.

### CROSS-REFERENCE TO RELATED APPLICATIONS

This application claims priority to and the filing date benefit of U.S. Provisional Patent Application No. 63/405,055, filed Sep. 9, 2022, and entitled “Systems and Methods for Administration of Drugs,” the disclosure of which is incorporated herein by reference in its entirety.

### BACKGROUND

The embodiments described herein relate to systems, apparatuses, applications, methodologies and other tools to administer medications and other treatments to patients experiencing an acute medical condition (e.g., cardiac arrest and trauma-induced hemorrhaging) as well as other prolonged acute and critical care management situations (e.g., trauma-related pain and burn relief, sedation, and ani-anxiety). In particular this disclosure relates to an automated medication administration system that includes an integrated cartridge assembly that is coupled to a pump assembly. The automated medication administration system is employed in response to the acute medical condition.

Heart disease is the leading cause of death worldwide with 17.3 million deaths each year. That number is expected to rise further by 2030 to 23.6 million. Conditions such as hypertension, hyperlipidemia, electrolyte imbalances, and trauma may lead to cardiac arrest where the patient's heart cannot provide adequate blood supply to vital organs, leading to severe injury or death. Cardiac arrest also compromises blood flow to the heart itself, leading to ischemia. Providing proper medical intervention soon after the onset of cardiac arrest is vital. In the United States, only about 10% of people who sustain a cardiac arrest survive. The survival rate triples when an arrest is witnessed by a bystander who can provide immediate assistance by administering cardiopulmonary resuscitation CPR and/or by summoning aid.

A “code” is a medical term used to describe a situation where a patient requires resuscitative efforts by a team of medical professionals, usually because the patient is experiencing cardiopulmonary arrest. Cardiopulmonary arrest may be due to various underlying causes resulting in an abnormal heartbeat or the absence of a heartbeat. Generally, treatments provided during a code focus on resuscitative efforts to restore a normal or near normal heartbeat to maintain blood flow throughout the body. Because vital organs and the central nervous system can be injured by interruption of blood flow for even short amounts of time, medical treatment during a code needs to be performed quickly. Often decisions to administer treatment during a code are complex and depend on the patient's medical condition, which may change from moment to moment.

Common interventions performed during a code include chest compressions (to compensate for a patient's heart not beating normally on its own), rescue breathing (to increase the level of oxygen in circulating blood), electrical shocks (to stimulate a patient's heart to beat normally), and delivery of various medications (to stimulate the heart or change the rhythm in which the heart is beating). Non-medical rescuers may learn skills focused on the first two of these interventions as part of Basic Life Support (BLS). Additionally, people trained in BLS may learn how to use an Automated External Defibrillator (AED), a device that interprets a patient's heart rhythm and potentially delivers an electrical shock based on that interpretation. BLS and use of an AED are based on established protocols. For example, BLS includes determining the patient's condition and administering chest compressions, rescue breathing, and/or electrical stimulation with an AED.

Medical professionals (e.g., emergency responders, nurses, pharmacists, physicians, nurse practitioners, physician assistants, etc.) may learn a more advanced form of intervention that, like BLS, is also largely protocol-based. For adult patients, this set of protocols or algorithms is known as Advanced Cardiac Life Support (ACLS). For children, this is known as Pediatric Advanced Life Support (PALS). ACLS and PALS focus on additional medication delivery and rescuer-selected electrical intervention based on a rescuer's interpretation of various cardiac rhythms. PALS is similar to ACLS but it utilizes weight-based medication dosages.

Code situations are often chaotic, with life-or-death decisions being made by a code leader and communicated to other professionals verbally and under time pressure. These circumstances can lead to errors in treatment. For example, the code leader's instructions regarding the medication, dosage, or route of administration may be misunderstood by team members, e.g., instructions to administer 1 mg of 1:1,000 concentration epinephrine instead of the appropriate 1:10,000 concentration. In addition, there may be errors in how medications are prepared or labeled, or which medication is actually administered because packaging or labeling of different medications look similar.

In accordance with the ACLS and PALS algorithms, specific doses of particular medications must be given at prescribed time intervals based upon the characteristics of the patient and patient's response to interventions. For example, under the ACLS protocol, epinephrine is administered in 1 mg doses every three to five minutes. Similarly, a first 300 mg dose of amiodarone is administered with a second 150 mg dose following, after a prescribed interval. Accordingly, it is desirable that the full prescribed dosage of the correct medication be administered to the patient at the prescribed intervals and that the full prescribed dose reach the patient, even under the chaos of the code situation. However, the time-constrained and rapidly evolving nature of a code situation may present challenges related to rapidly locating appropriate medications, manual dose calculations, therapeutic preparations, complete dose delivery, manual administration into an access port, proper adherence to a protocol sequence.

The time stress of a code situation also makes it difficult to create a record of what treatments were administered, at what time, and in the context of the patient's condition. In the case of cardiac arrest, the patient's condition may change rapidly. Decisions by the code leader and other code team members are made in response to the patient's condition and there may not be time to record what was done. Poor recordkeeping during the code may make it difficult for team members and other professionals to learn how to improve or to assess whether proper treatment was given. Additionally, poor recordkeeping can negatively impact the hand off of the patient between health care providers as such a hand off includes information about recently administered drugs, dosages, and timings.

Accordingly, there is a need for an automated medication administration system that is employed with to deliver drugs in response to the acute medical condition.

### SUMMARY

This summary introduces certain aspects of the embodiments described herein to provide a basic understanding. This summary is not an extensive overview of the inventive subject matter, and it is not intended to identify key or critical elements or to delineate the scope of the inventive subject matter.

In some embodiments, the present disclosure is directed to a pump assembly for delivering a medicament. The pump assembly includes a housing that has a wall. The wall includes an inner face that defines an internal volume. The wall includes a coupling interface positioned to engage a coupling member of a cartridge assembly. The cartridge assembly has a first container containing saline and a second container containing a medicament. The coupling interface includes a coupling protrusion positioned to engage an actuation portion of a gate member of the cartridge assembly. The engagement of the actuation portion by the coupling protrusion transitions the gate member from a locked position to an unlocked position. The pump assembly also includes a drive assembly positioned within the internal volume. The drive assembly includes a first drive member and a second drive member. The first drive member is operably coupled to the first container and configured to produce a first delivery force to convey a dose of saline from the first container when the gate member is in the unlocked position. The second drive member is operably coupled to the second container and configured to produce a second delivery force to convey a dose of the medicament from the second container when the gate member is in the unlocked position. The first drive member is actuatable independently of the second drive member.

In some embodiments, the first drive member is a first piston configured to exert the first delivery force on a first elastomeric member of the first container to convey the dose of saline, and the second drive member is a second piston configured to exert a second delivery force on a second elastomeric member of the second container to convey a dose of medicament.

In some embodiments, the cartridge assembly includes a manifold assembly in fluid communications with a tube set. The tube set is configured to be coupled to a medical port of a patient. The second delivery force exerted on the second elastomeric member is configured to convey the dose of the medicament from the second container to the patient via the manifold assembly and the tube set. The first delivery force exerted on the first elastomeric member is configured to flush the dose of the medicament from the manifold assembly, the tube set, and the medical port via the dose of saline from the first container.

In some embodiments, a delivery volume of the cartridge assembly is defined by a fluid volume of the manifold assembly, a fluid volume of the tube set, and the fluid volume of the medical port. The pump assembly includes a controller operably coupled to the drive assembly that includes at least one processor configured to perform a set of operations. The set of operations includes controlling the first delivery force such that a magnitude of the dose of saline is greater than or equal to 1.0 and less than or equal to 1.1 times the delivery volume.

In some embodiments, the drive assembly includes a first actuation member surrounding the first drive member and a second actuation member surrounding the second drive member, the first actuation member having at least one engagement lug positioned to be received by one of a set of actuation slots of the cartridge assembly. The engagement lug of the first actuation member is positioned to exert a first actuation force on a first container body of the first container in response to the pump assembly being actuated. The first actuation force causes the first container to be placed in fluid communication with a manifold assembly of the cartridge assembly. The second actuation member has at least one engagement lug positioned to be received by one of the set of actuation slots of the cartridge assembly. The engagement lug of the second actuation member is positioned to exert a second actuation force on a second container body of the second container in response to the pump assembly being actuated the second actuation force causing the second container to be placed in fluid communication with the manifold assembly.

In some embodiments, the first actuation force causes the first container to move into contact with a first puncturer of the cartridge assembly; and the second actuation force causes the second container to move into contact with a second puncturer of the cartridge assembly.

In some embodiments, a first energy storage device surrounds the first drive member. The first energy storage device is positioned between the wall and the first actuation member to generate the first actuation force in a distal direction when actuated. A second energy storage device surrounds the second drive member. The second energy storage device is positioned between the wall and the second actuation member to generate the second actuation force in the distal direction when actuated.

In some embodiments, the first energy storage device and the second energy storage device are positioned at least partially within corresponding recesses defined by the wall.

In some embodiments, the first drive member includes a first retention portion positioned to engage the first actuation member when the first drive member is in a ready state to establish a separation distance between the engagement lug of the first actuation member and the first container body. The second drive member includes a second retention portion positioned to engage the second actuation member when the second drive member is in the ready state to establish the separation distance between the engagement lug of the second actuation member and the second container body.

In some embodiments, the first drive member is configured to disengage from the first actuation member and is positioned to exert the first delivery force on a first elastomeric member of the first container when in an actuated state. The engagement lug of the first actuation member is configured to exert the first actuation force on the first container body when in the actuated state. The second drive member is configured to disengage from the second actuation member and is positioned to exert the second delivery force on a second elastomeric member of the second container when in the actuated state. The engagement lug of the second actuation member is configured to exert the second actuation force on the second container body when in the actuated state.

In some embodiments, the first energy storage device and the second energy storage device are positioned to bias the first container body and the second container body against the manifold assembly when in the actuated state.

In some embodiments, the cartridge assembly includes a manifold assembly configured to move in a proximal direction and into fluid communication with the first container and the second container in response to the gate member being moved to the unlocked position.

In some embodiments, the pump assembly includes a cap member sized to selectively occlude the coupling interface when the pump assembly is in a stored state.

In some embodiments, the coupling interface includes a latch assembly positioned to selectively couple the cartridge assembly to the pump assembly.

In some embodiments, the drive assembly includes a drive assembly chassis defining a set of guide slots extending parallel to a longitudinal axis of the pump assembly, a first motor, and a first lead screw supported by the drive assembly chassis and operably coupled to the first drive member. The first drive member includes a first piston guide block configured to be received by one of the set of guide slots. The drive assembly also includes a second motor and a second lead screw supported by the drive assembly chassis and operably coupled to the second drive member. The second drive member includes a second piston guide block configured to be received by one of the set of guide slots defined by the drive assembly chassis. Each of the first motor and the second motor define a motor axis that is parallel to the longitudinal axis. Each of the first lead screw and the second lead screw define a lead-screw axis that is parallel to the longitudinal axis and is displaced from the motor axis. Each guide slot of the set of guide slots is displaced from each motor axis and each lead screw axis.

In some embodiments, the pump assembly includes a set of longitudinal-position sensors coupled to the drive assembly chassis. The set of longitudinal-position sensors are positioned to monitor a longitudinal position of the first drive member and the second drive member.

In some embodiments, the pump assembly includes a load cell assembly positioned to monitor an axial load of the first lead screw and the second lead screw in a proximal direction.

In some embodiments, the pump assembly includes an output assembly configured to deliver a set of outputs to an operator of the pump assembly. The pump assembly also includes an input assembly configured to receive a set of inputs from the operator. Further, the pump assembly includes a controller operably coupled to the output assembly, the input assembly, and the drive assembly. The controller includes at least one processor configured to perform a set of operations. The set of operations includes determining a cartridge assembly classification of the cartridge assembly based on an input from a sensing element. The set of operations includes implementing a treatment protocol based on the cartridge assembly classification. The treatment protocol includes a medicament dosage and delivery schedule. The set of operations also includes generating at least one output of the set of outputs that corresponds to a dose-delivery prompt in accordance with the treatment protocol. The set of operations includes receiving a dose-delivery input from the operator. In response to the dose-delivery input, the set of operations includes delivering a first command signal to the drive assembly. The first command signal causes the second drive member to exert the second delivery force on a second elastomeric member of the second container to convey the dose of the medicament. Following the conveying of the dose of medicament, the set of operations includes delivering a second command signal to the drive assembly. The second command signal causes the first drive member to exert the first delivery force on a first elastomeric member of the first container to convey the dose of saline. Additionally, the set of operations includes delivering additional doses of the medicament in accordance with the treatment protocol followed by additional doses of saline.

In some embodiments, the set of operations includes recording a delivery time and dosage volume for each dose of the medicament delivered and generating an automated treatment record.

In some embodiments, the set of operations includes receiving a cartridge-status input from the sensing element. The set of operations also includes determining a deviation of the cartridge-status input from a cartridge-status design value. Further, the set of operations includes delivering an error signal to the output assembly in response to the deviation between the cartridge-status input and the cartridge-status design value.

In some embodiments, the set of operations includes delivering a third command signal to the drive assembly to implement a priming of the cartridge assembly.

In some embodiments, the pump housing has a ratio of a lateral length to a longitudinal length that is between 1:2.40 and 1:2.60.

In some embodiments, the present disclosure is directed to a method for administering a medication to a patient experiencing an acute medical condition. The method includes coupling a cartridge assembly to a pump assembly. The cartridge assembly has a gate member that maintains a separation between a manifold assembly and a first container and a second container when the gate member is in a locked position. The coupling of the cartridge assembly to the pump assembly causes the gate member to transition to an unlocked position that facilitates a fluid coupling between the manifold assembly and the first container and the second container. The method also includes removing a portion of a tube set from a coil recess defined by the cartridge assembly, the tube set including a tube coupled between the manifold assembly and a T-connector. The T-connector is coupled to a medical port, and the pump assembly is actuated to cause a first drive member of a drive assembly to exert a first delivery force on the first container to convey a dose of saline and a second drive member of the drive assembly to exert a second delivery force on the second container to convey a dose of a medicament. The dose of saline is delivered following the dose of the medicament.

In some embodiments, the present disclosure is directed to a latch assembly for selectively coupling a cartridge to a pump assembly. The latch assembly includes a latch arm movably coupled to a housing of the pump assembly. The latch arm includes an engagement portion configured to selectively engage a coupling member of the cartridge. The latch assembly also includes an energy storage device operably coupled to the latch arm and configured to produce a first movement of the engagement portion. The latch assembly includes a lock member movably coupled to the housing of the pump assembly. The lock member is movable between a locked configuration and an unlocked configuration. The lock member limits a second movement of the engagement portion when the lock member is in the locked configuration.

In some embodiments, the latch arm includes a support structure. The engagement portion is coupled to the support structure. The support structure defines a receiving portion. The receiving portion is coupled to the energy storage device and configured to receive a motive force from the energy storage device. The latch arm is movably coupled to the housing via a coupling portion of the support structure.

In some embodiments, the coupling portion defines a pivot axis.

In some embodiments, the support structure includes a first end portion and a second end portion. The engagement portion is coupled to the first end portion. The receiving portion is at the second end portion. The coupling portion is positioned along a length of the support structure between the engagement portion and the receiving portion.

In some embodiments, the support structure defines a first lever arm extending between the coupling portion and the first end portion. The support structure defines a second lever arm extending between the coupling portion and the second end portion. A length of the first lever arm is greater than a length of the second lever arm.

In some embodiments, the support structure includes a first end portion and a second end portion. The engagement portion is coupled to the first end portion. The coupling portion is at the second end portion. The receiving portion is positioned along a length of the support structure between the engagement portion and the coupling portion.

In some embodiments, the support structure includes a stop protrusion extending from the support structure. The stop protrusion is positioned to establish a maximal lateral position of the engagement portion via contact with the housing.

In some embodiments, the engagement portion includes a first planar surface and a second planar surface. A normal vector of the first planar surface intersects with a normal vector of the second planar surface. The first planar surface is positioned to engage a corresponding surface of the coupling member of the cartridge.

In some embodiments, the engagement portion is positioned at least partially between the housing and a portion of a coupling protrusion of the pump assembly.

In some embodiments, the energy storage device is coupled between the latch arm and the housing.

In some embodiments, on a condition in which the cartridge is separated from the pump assembly, the engagement portion is at a first lateral distance relative to a longitudinal axis of the pump assembly. On a condition in which the cartridge is partially received within a coupling interface of the pump assembly, the engagement portion is at a second lateral distance relative to the longitudinal axis of the pump assembly. The second lateral distance is less than the first lateral distance. On a condition in which the lock member is in the locked configuration, the engagement portion is at a third lateral distance relative to the longitudinal axis of the pump. The third lateral distance being greater than the second lateral distance.

In some embodiments, on a condition in which the engagement portion is in contact with a receiver structure of the coupling member of the cartridge and the lock member is in the unlocked configuration, the engagement portion is at a fourth lateral distance relative to the longitudinal axis of the pump assembly. The fourth lateral distance is less than the third lateral distance.

In some embodiments, on a condition that the engagement portion is at the fourth lateral distance, a surface of the engagement portion is in contact with an angular surface of the receiver structure to develop a retention force. The retention force has a magnitude that is greater than a weight of the cartridge.

In some embodiments, a transition from the fourth lateral distance to the third lateral distance corresponds to a longitudinal movement of the cartridge within the coupling interface.

In some embodiments, the lock member is a helical wedge. The movement of the lock member between the locked configuration in the unlocked configuration includes a rotation of the helical wedge.

In some embodiments, the helical wedge rotates around an axis of rotation that is parallel to one of a lateral axis or a longitudinal axis of the pump assembly.

In some embodiments, the latch assembly includes a motor operably coupled to the lock member. The motor is configured to move the lock member between the locked configuration and the unlocked configuration.

In some embodiments, the lock member is separated from the cartridge when the lock member is in the locked configuration, the unlocked configuration, and a transition therebetween.

In some embodiments, the latch arm is a first latch arm, the engagement portion is a first engagement portion, and the movement is in a first lateral direction. The latch assembly includes a second latch arm movably coupled to the housing of the pump assembly. The second latch arm includes a second engagement portion configured to selectively engage the coupling member of the cartridge. The second latch arm is movable in a second lateral direction via the energy storage device. The second lateral direction being opposite the first lateral direction. The lock member limiting the movement of the second engagement portion when the lock member is in the locked configuration.

In some embodiments, the energy storage device is coupled between the first latch arm and the second latch arm.

In some embodiments, a displacement of the first engagement portion in the first lateral direction is substantially equal to a displacement of the second engagement portion in the second lateral direction.

In some embodiments, an angular separation between the first engagement portion and the second engagement portion has a first angular magnitude on a condition in which the cartridge is separated from the pump assembly. On a condition in which the cartridge is partially received within a coupling interface of the pump assembly, the angular separation has a second angular magnitude that is less than the first angular magnitude. Additionally, on a condition in which the lock member is in the locked configuration, the angular separation has a third angular magnitude that is greater than the second angular magnitude.

In some embodiments, on a condition in which the engagement portion is in contact with a receiver structure of the coupling member of the cartridge and the lock member is in the unlocked configuration, the angular separation has a fourth angular magnitude that is less than the third angular magnitude.

In some embodiments, on a condition that the angular separation has the fourth angular magnitude, the first engagement portion and the second engagement portion are in contact with the receiver structure to develop a retention force; and the retention force has a magnitude that is greater than a weight of the cartridge.

In some embodiments, an increase in the angular separation from the fourth angular magnitude to the third angular magnitude corresponds to a longitudinal movement of the cartridge within the coupling interface in a proximal direction.

In some embodiments, the present disclosure is directed to a pump assembly for delivering a medicament. The pump assembly includes a pump housing that has a coupling interface configured to receive a cartridge assembly and a drive assembly positioned within the pump housing and configured to cause delivery of a dose of a medicament from the cartridge assembly positioned at a specified longitudinal location relative to the pump housing. Additionally, the pump assembly includes a latch assembly positioned to selectively secure the cartridge assembly within the coupling interface. The latch assembly has a passive lock mechanism and an active lock mechanism. The passive lock mechanism is configured to retain the cartridge assembly within the coupling interface while permitting a removal of the cartridge assembly in response to an external force applied to the cartridge assembly. The active lock mechanism is configured to secure the cartridge assembly at the specified longitudinal location and preclude removal of the cartridge assembly from the coupling interface.

In some embodiments, the passive lock mechanism includes an energy storage device operably coupled to a latch arm that has an engagement portion. The energy storage device is configured to establish the engagement portion in contact with a receiver structure of the cartridge assembly to develop a retention force. The retention force has a magnitude that is greater than a weight of the cartridge assembly.

In some embodiments, the latch assembly includes a latch arm that has an engagement portion and a lock member that is movably coupled to the pump housing. The lock member is movable between a locked configuration and an unlocked configuration. The lock member is separated from the cartridge assembly in the locked configuration, the unlocked configuration, and a transition therebetween. On a condition that the lock member is in the unlocked configuration, the cartridge assembly is retained within the coupling interface via the passive lock mechanism. The lock member maintains the engagement portion in contact with a receiver structure of the cartridge assembly on a condition that the lock member is in the locked configuration.

In some embodiments, a transition of the lock member from the unlocked configuration to the locked configuration develops a longitudinal movement of the cartridge assembly within the coupling interface in a proximal direction. The longitudinal movement positions the cartridge assembly at the specified longitudinal location.

### BRIEF DESCRIPTION OF THE DRAWINGS

FIG. 1 is a perspective view of an acute medical condition response system in an assembled configuration according to an embodiment.

FIG. 2 is a perspective view of the system of FIG. 1 with the cartridge assembly separated from the pump assembly and a removable cap installed.

FIG. 3 is a perspective view of the system of FIG. 1 with the cartridge assembly separated from the pump assembly.

FIG. 4 is a side view of the system of FIG. 1.

FIG. 5 is a perspective view of the pump assembly of FIG. 3.

FIG. 6 is a cross-sectional view of the pump assembly of FIG. 3.

FIG. 7 is a perspective view of the cartridge assembly of FIG. 2.

FIG. 8A is a front view of the cartridge assembly of FIG. 2.

FIG. 8B is a rear view of the cartridge assembly of FIG. 2.

FIG. 9 is an exploded perspective view of the cartridge assembly of FIG. 2.

FIG. 10 is a perspective view of a set of cartridges of the cartridge assembly according to an embodiment.

FIG. 11 is a side view of the cartridge assembly of FIG. 2 in a storage configuration (i.e., prior to being coupled to the pump), with a portion of the cartridge housing removed.

FIG. 12 is a cross-sectional view of a portion of the system of FIG. 1, showing the cartridge assembly in a first (i.e., loaded) configuration (coupled to the pump, but prior to actuation).

FIG. 13 is a side view of the cartridge assembly of FIG. 2 in a second configuration (i.e., an actuated configuration, after the cartridges are in fluid communication with the manifold assembly), with a portion of the cartridge housing removed.

FIG. 14A is a diagrammatic illustration of a portion of the system of FIG. 1 showing the cartridge assembly in a first (i.e., loaded) configuration (coupled to the pump, but prior to actuation).

FIG. 14B is a diagrammatic illustration of a portion of the system of FIG. 1 showing the cartridge assembly in a second configuration (i.e., an actuated configuration, after the cartridges are in fluid communication with the manifold assembly).

FIG. 14C is a diagrammatic illustration of a portion of the system of FIG. 1 showing the cartridge assembly in a third configuration (i.e., a dispensed configuration).

FIG. 15 is a perspective view of a drive assembly of pump assembly of FIG. 5.

FIG. 16 is a perspective view of a drive assembly of pump assembly of FIG. 5 with a gear cover removed.

FIG. 17 is a perspective view of a drive assembly of pump assembly of FIG. 5 with a portion of the drive members removed.

FIG. 18 is a perspective view of a drive assembly chassis of the drive assembly of FIG. 15.

FIG. 19 is a perspective view of a drive member of the drive assembly of FIG. 15.

FIG. 20 is an end view of an actuation member of the drive assembly of FIG. 15.

FIG. 21 is a perspective view of a tube set of the cartridge assembly of FIG. 2 according to an embodiment.

FIG. 22 is a perspective view of the tube set of FIG. 21 illustrating the tube in an uncoiled orientation.

FIG. 23 is a system diagram of the system of FIG. 1.

FIG. 24 is a flow chart of operations implemented by controller of the system of FIG. 1.

FIGS. 25-40 are graphical depictions of multiple outputs delivered to an operator of the system of FIG. 1.

FIG. 41 is a perspective view of an acute medical condition response system in an assembled configuration according to an embodiment.

FIG. 42 is a perspective view of the pump assembly of the system of FIG. 41.

FIG. 43 is a perspective view of the pump assembly FIG. 41 with a protrusion of the coupling interface removed to show underlying features.

FIG. 44 is a perspective view of the cartridge assembly of the system of FIG. 41.

FIG. 45 is a top view of the cartridge assembly of FIG. 44 in a storage configuration (i.e., prior to being coupled to the pump), with a portion of the cartridge housing removed.

FIG. 46 is a perspective view of the cartridge assembly of FIG. 44 in an actuated configuration, after the cartridges are in fluid communication with the manifold assembly), with a portion of the cartridge housing removed.

FIG. 47 is a schematic illustration of a controller for use with an acute medical condition response system according to an embodiment.

FIG. 48 is a flow chart of a method for administering a medication to a patient experiencing an acute medical condition.

FIG. 49 is a schematic illustration of a pump assembly with a latch assembly according to an embodiment, in a first configuration.

FIG. 50 is a schematic illustration of the pump assembly with the latch assembly of FIG. 49 in a second configuration.

FIG. 51 is a schematic illustration of the pump assembly with the latch assembly of FIG. 49 in a fourth configuration.

FIG. 52 is a schematic illustration of the pump assembly with the latch assembly of FIG. 49 in a third configuration.

FIG. 53 is an exploded top view of a latch assembly of the pump assembly, according to an embodiment.

FIG. 54 is a top view of latch arms of the latch assembly of FIG. 53 depicting a first angular magnitude and a second angular magnitude.

FIGS. 55A and 55B depict a lock member of the latch assembly of FIG. 53 in an unlocked configuration and a locked configuration, respectively.

FIG. 56 is a top view of a cartridge separated from a pump assembly according to an embodiment. A portion of the pump assembly is removed for clarity.

FIG. 57 is a top view of the cartridge partially received within the pump assembly of FIG. 56.

FIG. 58 is a top view of the cartridge partially received within the pump assembly of FIG. 56 and retained via a passive lock mechanism according to an embodiment.

FIG. 59 is a top view of the cartridge assembly at a designated longitudinal position with the lock mechanism of the latch assembly of FIG. 53 in a locked configuration according to an embodiment.

### DETAILED DESCRIPTION

Generally, the present disclosure is directed to systems and methods for administering medication in response to an acute medical condition. As disclosed herein, the acute medical condition response system (also referred to as an Automated Medication Administration System (AMAS)) is a drug-device combination product designed to automatically deliver at least one drug followed by a portion of saline from an on-board supply or another drug in response to specified acute medical conditions. For example, the system can be configured to automatically deliver epinephrine and/or amiodarone during cardiopulmonary resuscitation (CPR) of adults and pediatric victims of cardiac arrest. The system will deliver the drugs according to the dosing and timing recommended in established treatment protocols (e.g., the American Heart Association's algorithms for treatment of adult and pediatric cardiac arrest). The system is intended to be used in both out-of-hospital and in-hospital settings, to enable a shorter time to administer of the drugs, ensure accurate dosing, keep automated records of the dose, and eliminate the human delay and medication error that may result from drawing up from a vial, and/or use of multiple syringes.

The system can be of particular benefit in pediatric cases where drugs are dosed based on the patient's weight. For example, the target dose for pediatric use can be weight-based at 5 mg/kg, with patients weighing between 3 kg and 30 kg, inclusive. In such a case, the target volume of an amiodarone hydrochloride injection to be delivered over this pediatric weight range ranges from 0.3 mL to 3 mL, based on the weight-based pediatric 5 mg/kg dose. As such, the system described herein is configured to facilitate the accurate dispensing of relatively small volumes (e.g., 3 mL). The system also employs an on-board saline flush to ensure that the entirety of the dispensed volume is delivered to the patient.

As described herein, the system includes a cartridge assembly (also referred to as a “cartridge”) that is coupled to a pump assembly and is also coupled to a medical port (e.g., an intravenous (IV) catheter, an IV cannula, a central venous access device, an intraosseous (IO) access device, an implanted port, a peripherally inserted central catheter (a PICC line), or other medical implement for facilitating access to the circulatory system of the patient). The cartridge assembly contains at least two color-coded cartridges. One cartridge contains a medicament, such as epinephrine or amiodarone hydrochloride, while the other cartridge contains a sodium chloride (saline) solution. Each of the cartridges are sealed with a frangible seal when the cartridge assembly is in a stored state. When actuated, the frangible seals are punctured by a number of puncturers (e.g., needles) of a manifold assembly within the cartridge assembly. This actuation places the cartridges in fluid communication with the medical port (e.g., a vascular access device) via a tube set that has a specified volume. The pump assembly can then deliver a dose of the medicament in accordance with the protocol being implemented. This is followed by delivering a portion of the saline from the cartridge assembly. By following the medicament with the portion of saline, the system ensures that the entirety of the prescribed dose is flushed from the tube set and delivered accurately to the patient. This process can be repeated in accordance with the protocol.

As further described herein, the cartridge assembly is a sealed system in which the cartridges are not accessible to the user. Thus, the cartridges are prefilled with multiple doses at the desired concentration and formulation of medicament and are ready for automated delivery without the need for the user to prepare a delivery syringe with a desired type and amount of medicament. A viewing port in the housing allows intended users to visually inspect the cartridges (e.g., glass cartridges) within the cartridge assembly. In some embodiments, the cartridge assembly contains three individual glass cartridges that contain epinephrine, amiodarone HCl, and sodium chloride, respectively. The cartridges utilize colored aluminum crimp seals for each cartridge to ensure differentiation and inspection. The cartridge assembly also includes a sensor interface (e.g., an RFID tag) that is readable by a sensor of the pump assembly and indicates whether the cartridge is configured for an adult patient or a pediatric patient. The sensor interface can also indicate other information about the cartridge, such as an expiration date, a manufacture date, lot information, a storage compliance indication, a recall status, and/or a prior actuation of the cartridge. For example, the sensor interface may indicate that a cartridge has been previously used and the system may generate an error.

The utilization of the acute medical condition response system reduces the cognitive load of the care providers when responding to an acute medical condition by automating aspects of medication selection, dosage delivery, and adherence to a dosage schedule. This, in turn, facilitates the focus of the care providers on treating reversible causes. For example, each cartridge assembly contains sufficient dosages of the prescribed drug to comply with the dosage schedule of the implemented protocol and the pump assembly facilitates the delivery of the prescribed dose. Additionally, the utilization of the tube set having a prescribed volume in conjunction with the bolus of saline from the saline cartridge ensures the accuracy of the prescribed dose that is delivered to the patient. This may be especially beneficial to ensure accuracy for the relatively small volumes corresponding to pediatric patients.

As used herein, the term “about” when used in connection with a referenced numeric indication means the referenced numeric indication plus or minus up to 10 percent of that referenced numeric indication. For example, the language “about 50” covers the range of 45 to 55. Similarly, the language “about 5” covers the range of 4.5 to 5.5.

As used herein, the terms “proximal” and “distal” refer to direction closer to and away from, respectively, an operator of the system. Thus, for example, the end of the cartridge assembly closest to or contacting the patient's body would be the distal end of the cartridge assembly, while the end opposite the distal end (i.e., the end closer to the operator) would be the proximal end of the system.

Further, specific words chosen to describe one or more embodiments and optional elements or features are not intended to limit the invention. For example, spatially relative terms—such as “beneath”, “below”, “lower”, “above”, “upper”, “proximal”, “distal”, and the like—may be used to describe the relationship of one element or feature to another element or feature as illustrated in the figures. These spatially relative terms are intended to encompass different positions (i.e., translational placements) and orientations (i.e., rotational placements) of a device in use or operation in addition to the position and orientation shown in the figures. For example, if a device in the figures is turned over, elements described as “below” or “beneath” other elements or features would then be “above” or “over” the other elements or features. Thus, the term “below” can encompass both positions and orientations of above and below: A device may be otherwise oriented (e.g., rotated 90 degrees or at other orientations) and the spatially relative descriptors used herein interpreted accordingly. Likewise, descriptions of movement along (translation) and around (rotation) various axes includes various spatial device positions and orientations.

Similarly, geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

In addition, the singular forms “a”, “an”, and “the” are intended to include the plural forms as well, unless the context indicates otherwise. The terms “comprises”, “includes”, “has”, and the like specify the presence of stated features, steps, operations, elements, components, etc. but do not preclude the presence or addition of one or more other features, steps, operations, elements, components, or groups.

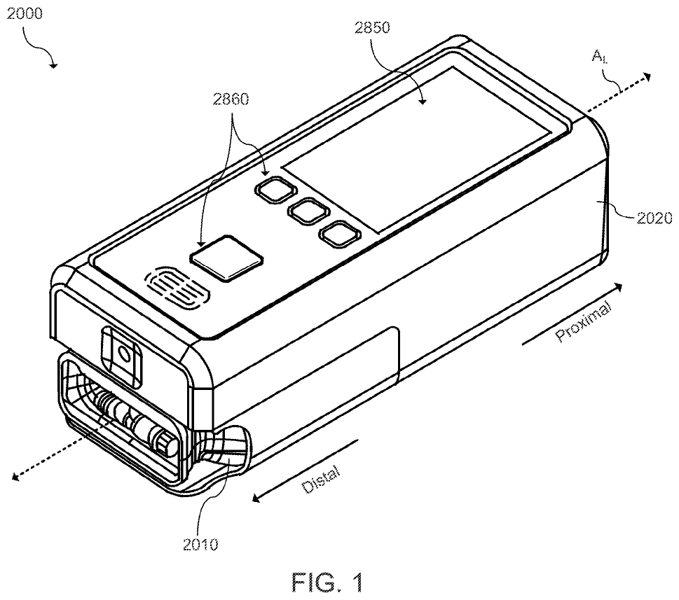

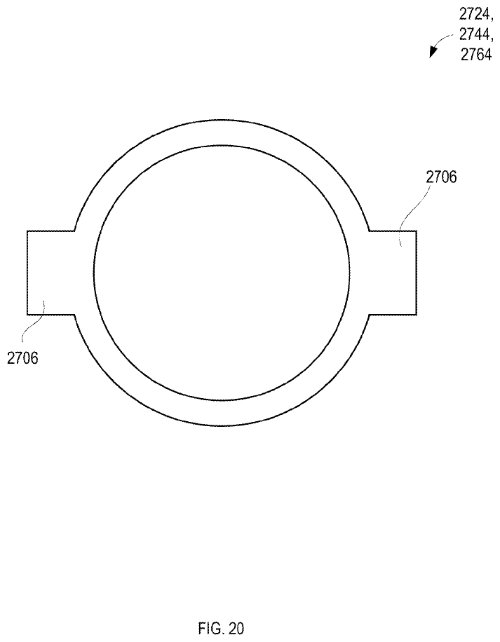

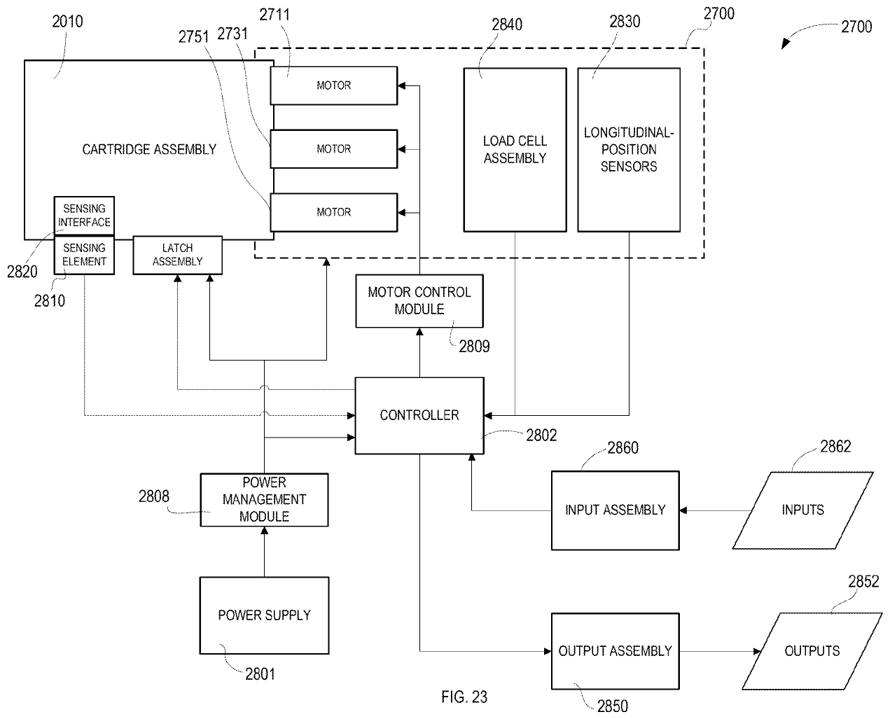

FIGS. 1-40 depict various aspects of an acute medical condition response system (system) (also referred to as an Automated Medication Administration System (AMAS) or “system”) 2000. As described herein, the system 2000 is configured to automate certain aspects of a response to an acute medical condition, such as cardiac arrest. Accordingly, the system 2000 utilizes a sealed cartridge assembly 2010 containing more than one dose of a drug (e.g., epinephrine, amiodarone hydrochloride, or other desired medicament) and more than one dose of a separate saline solution. The cartridge assembly 2010 is configured to be operably coupled between a pump assembly 2020 and a medical port (not shown) that is inserted into or attached to a patient. In this manner, the system 2000 can be used with a conventional medical port.

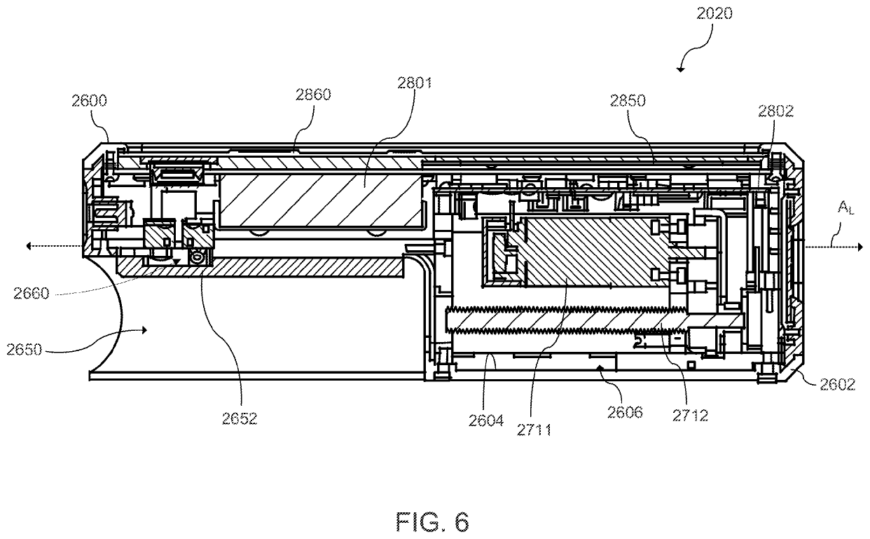

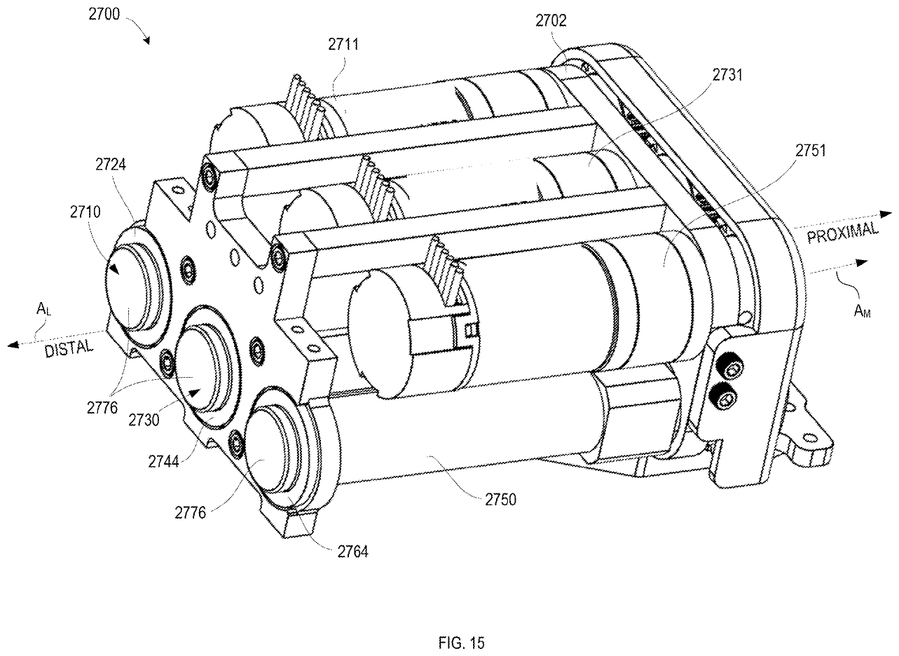

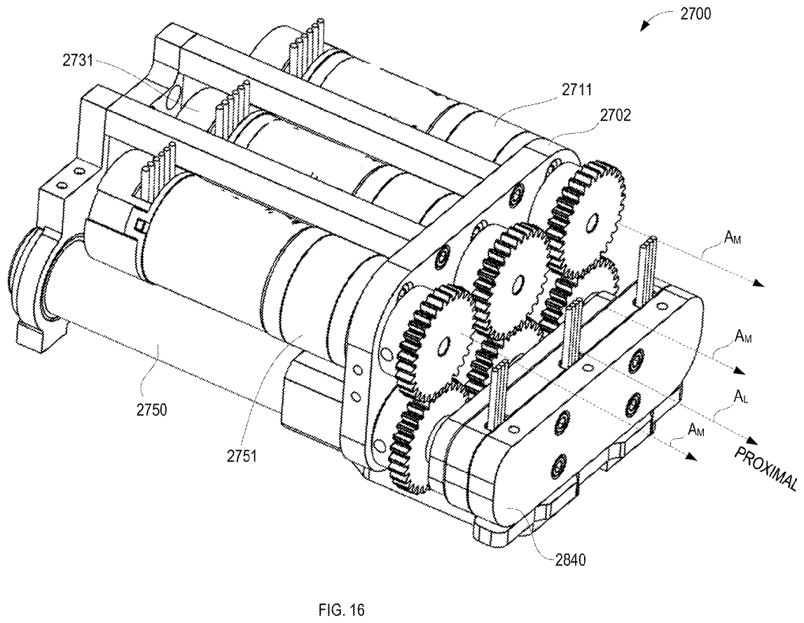

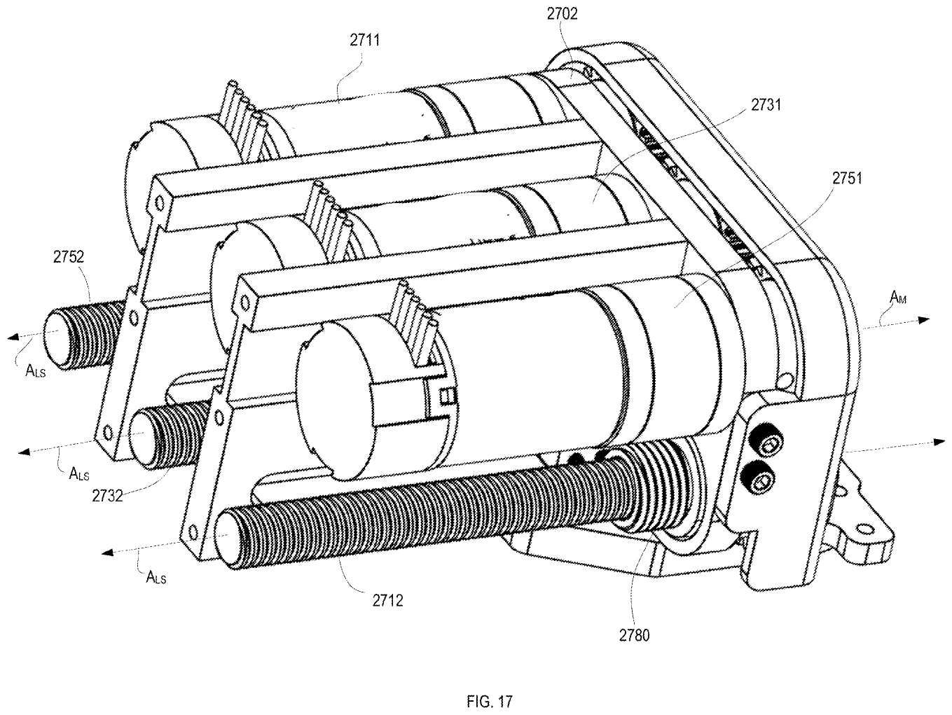

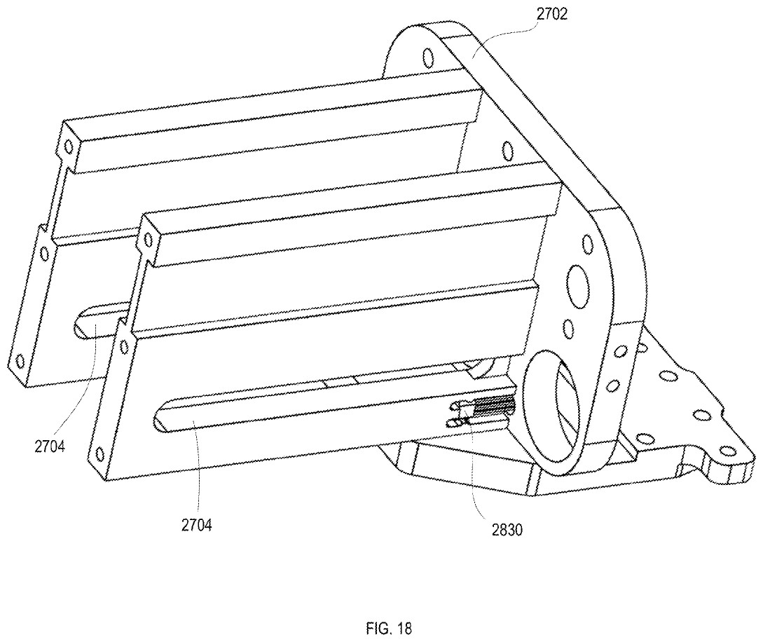

As depicted in FIG. 6, the pump assembly 2020 includes a pump housing 2600 having a wall 2602. The wall 2602 includes an inner face 2604 that defines an internal volume 2606. The wall 2602 includes a coupling interface 2650. The coupling interface 2650 is positioned to engage a coupling member 2130 of the cartridge assembly 2010. The coupling interface 2650 includes a coupling protrusion 2652 that is positioned to engage and actuation portion 2510 of the gate member 2500 of the cartridge assembly 2010. The engagement of the actuation portion 2510 by the coupling protrusion 2652 transitions the gate member 2500 from a locked position P_{L} to an unlocked position P_{U}. The transition of the gate member 2500 facilitates the actuation of the cartridge assembly 2010 and the subsequent delivery of at least one dose of medicament in response to the acute medical condition. Accordingly, the pump assembly 2020 also includes a drive assembly 2700 (FIGS. 15-20) that is positioned within the internal volume 2606. The drive assembly 2700 includes at least a first drive member 2710 and a second drive member 2730. The first drive member 2710 is operably coupled to the first container 2210 and configured to produce a first delivery force F_{D} (FIGS. 14B and 14C) to convey a dose of saline from the first container 2210 when the gate member 2500 is in the unlocked position P_{U}. Similarly, the second drive member 2730 is operably coupled to the second container 2230 and configured produce a second delivery force F_{D} to convey a dose of the medicament from the second container 2230 when the gate member 2500 is in the unlock position Up. The first drive member 2710 is actuatable independently of the second drive member 2730.

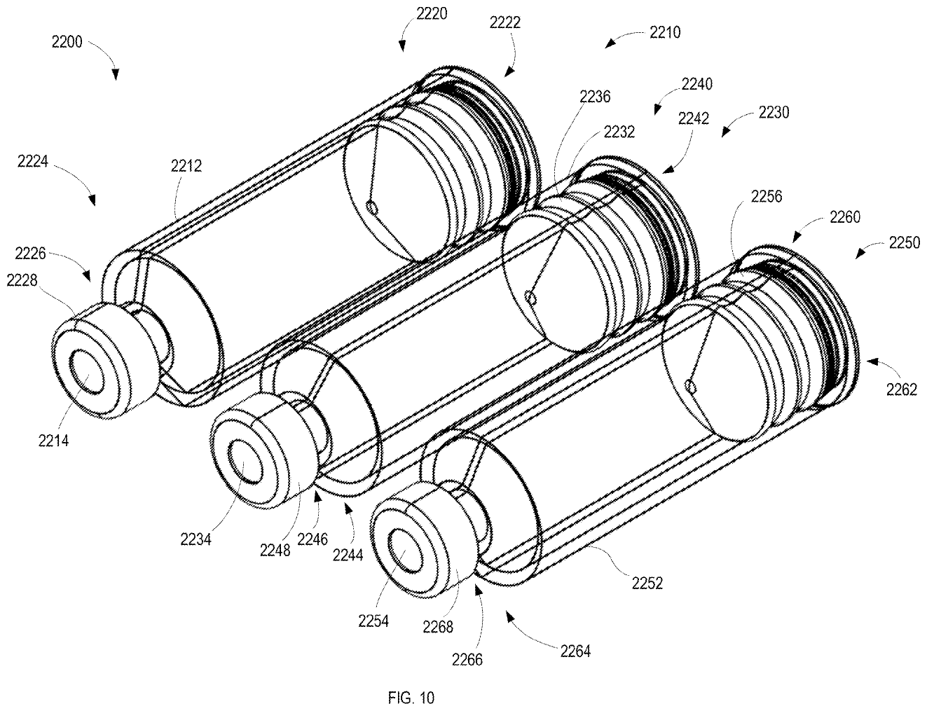

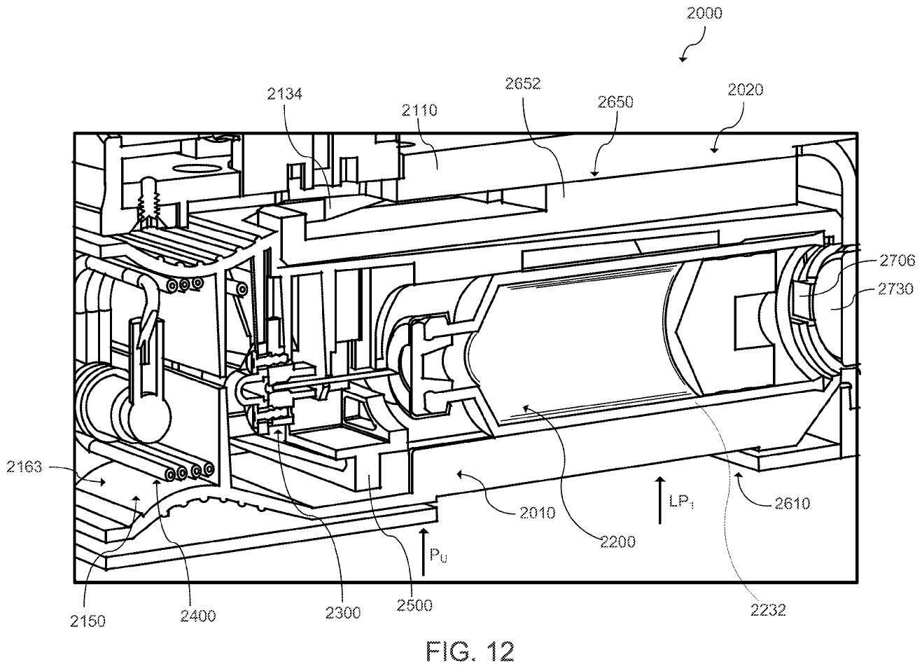

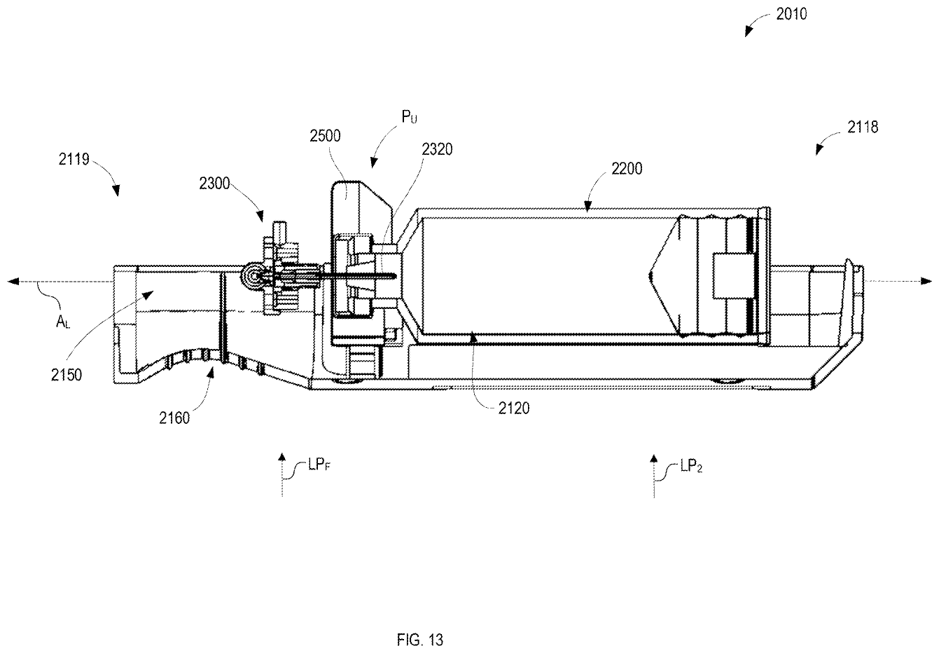

The cartridge assembly 2010 includes a housing 2100 having a wall that defines an internal volume 2120 (see FIG. 9) and includes a coupling member 2130. The coupling member 2130 is positioned to engage a coupling interface 2650 of the pump assembly 2020. A set of cartridges 2200 is positioned within the internal volume 2120. In this embodiment, the cartridge assembly 2010 includes a first container 2210 that contains a portion of saline, a second container 2230 that contains a first medicament, and a third container 2250 that contains a second medicament. A manifold assembly 2300 is also positioned within the internal volume 2120. As depicted in FIGS. 9 and 11-14, the manifold assembly 2300 includes a set of puncturers 2320 oriented to fluidically couple each of the cartridges 2200 to a tube set 2400 when the cartridge assembly 2010 is actuated. However, a gate member 2500 is also positioned within the internal volume 2120 to maintain a separation distance SD (see FIG. 11) between the set of puncturers 2320 and the set of cartridges 2200 when the cartridge assembly 2010 is in either a storage configuration as depicted in FIG. 11 or in a first (or loaded) configuration as depicted in FIG. 12. In this manner, the gate member 2500 functions as a safety to prevent the undesired or premature actuation of the cartridge assembly 2010.

In some embodiments, the cartridge assembly 2010 can be stored in a sterile package (not shown) until an acute medical condition is encountered. The sterile package can include a peel-away seal or a perforation for opening. In some embodiments, the sterile package can be constructed from a porous polymer that is formulated to allow a sterilant gas or substance to pass therethrough, while substantially preventing pathogens or microbes from passing therethrough. In this manner, the cartridge assembly 2010 can be sterilized after manufacture. In some embodiments, the sterile package can be constructed from any one of polyethylene, high-density polyethylene (i.e., Tyvek®), polypropylene, polytetrafluoroethylene, or thermoplastic polyurethane. The sterile package can be used with any suitable sterilization technique or medium, including ethylene oxide, gamma radiation, e-beam radiation, ultraviolet radiation, steam, plasma, or hydrogen peroxide. In some embodiments, the peel-away seal or the perforated region can include tamper indicating features (e.g., a feature that changes color if opened, a non-resealable feature, or the like). Markings may be applied to the packaging to indicate whether the cartridge assembly 2010 is an adult cartridge assembly or a pediatric cartridge assembly. The markings may also provide additional information related to the manufacture and/or usage of the cartridge assembly 2010. In some embodiments, a desiccant or other additives can be included in the packaging.

Referring to FIG. 9, the cartridge assembly 2010 includes a first wall piece 2110 and a second wall piece 2111 that are coupled together to form a wall of the housing 2100. In some embodiments, the first wall piece 2110 is formed as an upper wall segment extending between a proximal portion 2118 and a distal end portion 2119 of the housing 2100. Similarly, the second wall piece 2111 can be formed as a lower wall segment extending between the proximal portion 2118 and the distal end portion 2119 of the housing 2100. The wall formed by the first wall piece 2110 and a second wall piece 2111 includes an inner face 2112. The inner face 2112 defines the internal volume 2120. In some embodiments, the internal volume 2120 can include a first internal volume portion configured to receive the cartridges 2200. Additionally, the internal volume 2120 can include a second internal volume portion that may be positioned proximally of the first internal volume portion to receive the manifold assembly 2300 and/or the gate member 2500. The wall also defines a coil recess 2150 that contains the tube set 2400 when the cartridge assembly 2010 is in a stored state.

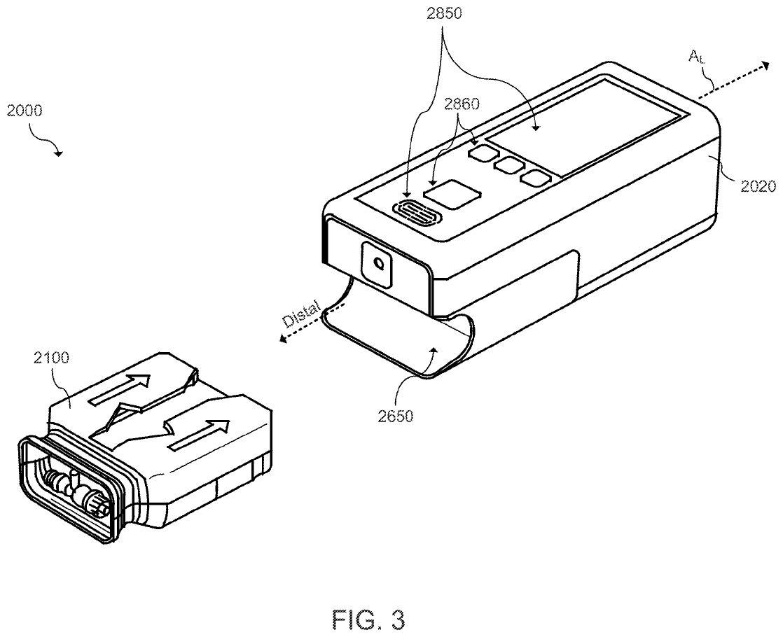

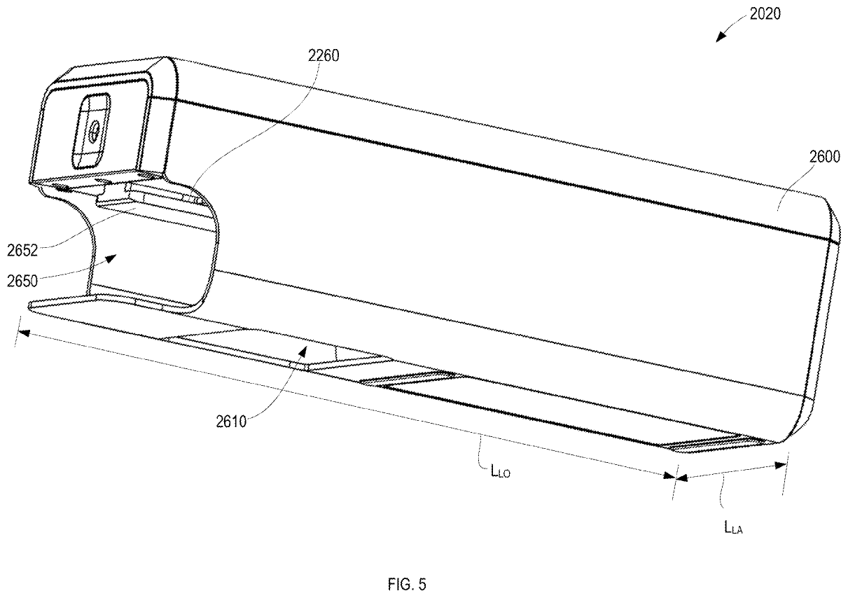

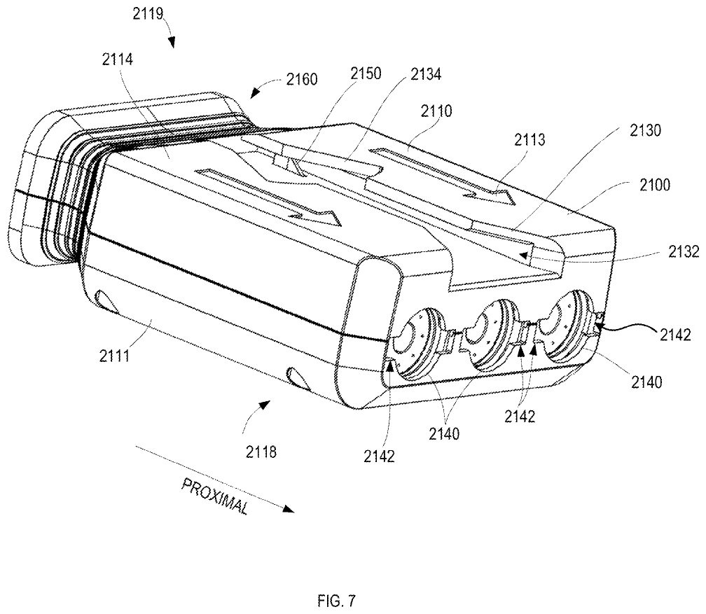

As depicted in FIG. 7 for example, an outer face 2114 of the wall (e.g., of the first wall piece 2110) includes the coupling member (or feature) 2130. The coupling member 2130 includes a keyway 2132 and a receiver structure 2134. The keyway 2132 is configured to receive a mating coupling protrusion 2652 (see, e.g., the coupling protrusion 2652 shown in in the pump of FIG. 5 configured as a T-track) of the pump assembly 2020. The keyway 2132 includes a tapered opening (at the proximal end portion 2118 of the housing 2100) to facilitate easy coupling to the pump assembly 2020. The keyway 2132 also includes shoulders that are coupled within corresponding channels defined by the coupling protrusion 2652 of the coupling interface 2650 to limit movement of the cartridge assembly 2010 relative to the pump assembly 2020 at least in any direction normal to the longitudinal axis A_{L} of the system 2000 (FIG. 3). Similarly stated, the keyway 2132 can ensure that access orifices 2140 are aligned with corresponding drive members 2710, 2730, 2750 (FIG. 15) of the pump assembly 2020. The keyway 2132 also ensures alignment of actuation slots 2142 with the corresponding actuation members 2724, 2744, 2764 (FIG. 15) of the pump assembly 2020. In this manner, when the cartridge assembly 2010 is coupled to the pump assembly 2020, it will be aligned to facilitate accurate and repeatable actuation and delivery of medicament. The receiver structure 2134 includes two angular features having intersecting shoulders that are configured to mate with a latch member 2660 (FIG. 6) of the pump assembly 2020. Thus, when the cartridge assembly 2010 is coupled to the pump assembly 2020, the latch member 2660 of the pump assembly 2020 will move and engage the receiver structure 2134. This engagement limits movement of the cartridge assembly 2010 relative to the pump assembly 2020 in a direction parallel to the longitudinal axis A_{L} of the system 2000. In this manner, the axial position of the cartridge assembly 2010 can be fixed relative to the pump assembly 2020 during use, which enhances the accuracy of the dose delivered. In some embodiments, the outer face 2114 can include various markings 2113, such as arrows indicating a direction in which the cartridge assembly 2010 is moved to couple the cartridge assembly 2010 to the pump assembly 2020.

The wall formed by the first wall piece 2110 and the second wall piece 2111 defines a maximal circumference of a cross-section that is perpendicular to the longitudinal axis A_{L}. The distal end portion 2119 of the wall defines a neck region 2160. The neck region 2160 has a cross-sectional circumference that is less than the maximal circumference. Said another way, the neck region 2160 is concave relative to the proximal portion 2118 of the housing 2100. The neck region 2160 includes a set of grip enhancement features that can include ridges, textures, bumps, depressions configured to facilitate handling of the cartridge assembly 2010 by an operator.

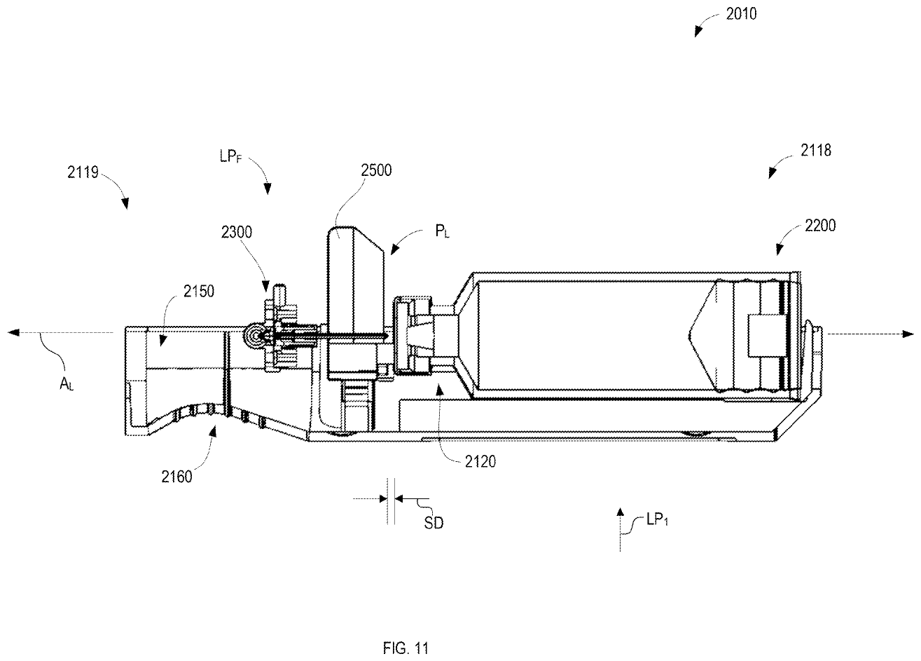

In some embodiments, the cartridge housing 2100 is formed with multiple sets of cradles sized and positioned to at least partially surround the plurality of cartridges 2200. As such, the cradles can align and/or secure the plurality of cartridges 2200. Moreover, the set of cradles at the proximal end portion 2118 define a portion of a boundary of the access orifices 2140. Thus, when the first wall piece 2110 is coupled to the second wall piece 2111, the housing 2100 defines the set of access orifices 2140 that are aligned with the elastomeric members of each cartridge (e.g., the elastomeric members 2216, 2236, 2256 as shown in FIG. 10). Additionally, the first wall piece 2110 can include such other tabs, protrusions, rails and/or guides as are required to support any other components of the cartridge assembly 2010 positioned within the internal volume 2120.

In some embodiments, the second wall piece 2111 is formed with a plurality of tapered ribs. Each of the tapered ribs is deformable. Accordingly, the plurality of tapered ribs is deflected from a neutral position by the cartridges 2200 when the second wall piece 2111 is joined to the first wall piece 2110 during assembly of the cartridge assembly 2010. This deformation of the plurality of tapered ribs results in a force applied to each of the cartridges 2200 thereby securing the cartridges 2200 during transport and storage of the cartridge assembly 2010. The proximal end portion 2118 of the second wall piece 2111 also defines a portion of a boundary of the access orifices 2140 and the actuation slots 2142. Thus, when the first wall piece 2110 is coupled to the second wall piece 2111, the housing 2100 defines the set of access orifices 2140 that are aligned with the elastomeric members of each cartridge 2200. Additionally, the second wall piece 2111 can be formed with a status opening that can be utilized by a user of the cartridge assembly 2010 to verify the contents of the cartridge assembly 2010 before or after employment.

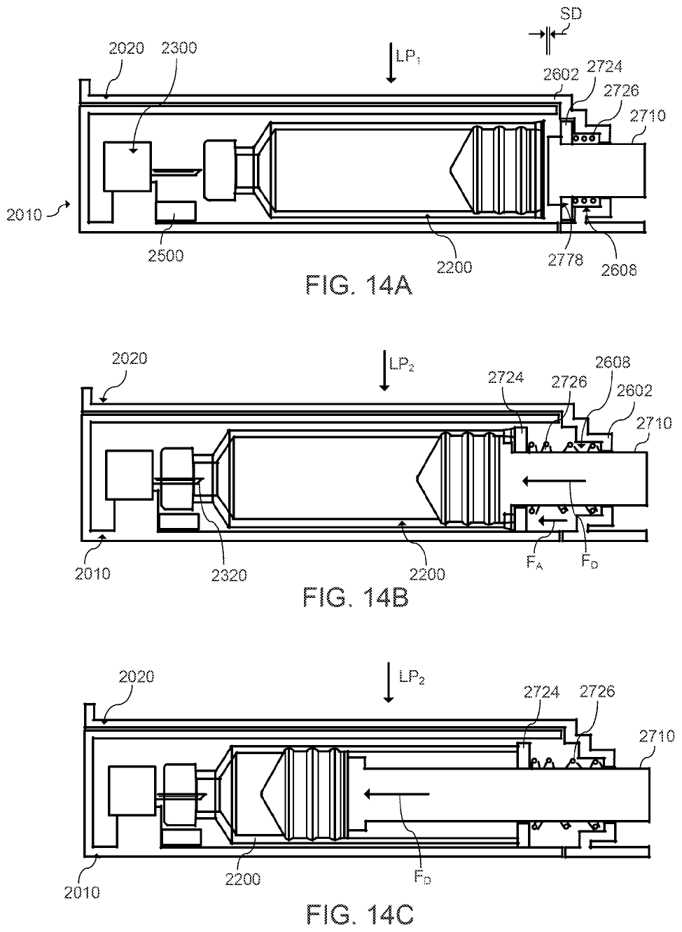

The housing 2100 also defines the actuation slots 2142 that can allow the actuation members 2724, 2744, 2764 of the pump assembly 2020 to exert a force on the container body (e.g., the first container body 2212, the second container body 2232, and/or the third container body 2252) of each cartridge 2200. In this manner, in some embodiments, a drive assembly 2700 of the pump assembly 2020 can exert an actuation force to move the cartridges 2200 within the housing 2100 (to place the cartridges in fluid communication with the manifold assembly 2300 that is independent from a delivery force that is exerted on the elastomeric member of each cartridge 2200 to cause delivery of the medicament therefrom. In some embodiments, the actuation force transition the cartridges 2200 from a first longitudinal position LP₁ (See, e.g., FIG. 11) to a second longitudinal position LP₂ (See, e.g., FIG. 13) that is distal of the first longitudinal position LP₁ when the cartridge assembly 2010 is in an actuated state (also referred to as a second configuration).

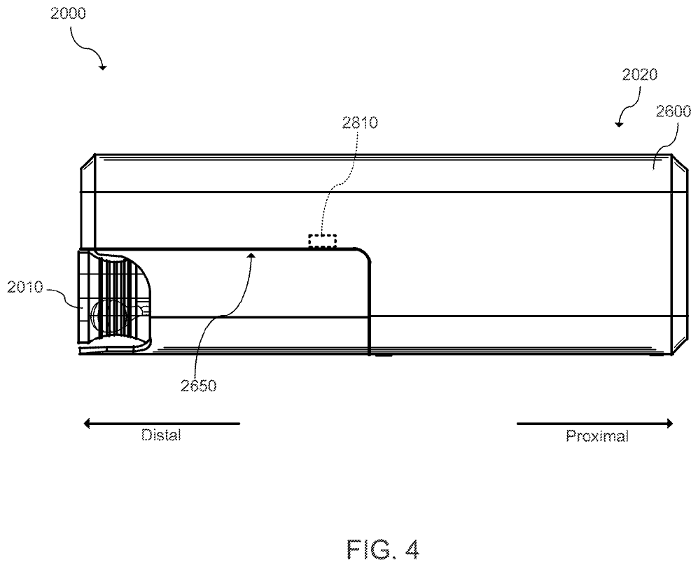

In some embodiments, the first wall piece 2110 can also define and/or support a sensor interface 2820 (FIG. 9) for engaging with a sensing element 2810 (FIG. 4) of the pump assembly 2020. For example, the first wall piece 2110 can be formed with a recess into which the sensor interface 2820 may be inserted. The sensor interface 2820 can be any suitable feature that can operably engage with the sensing element, such as, for example, an RFID tag, an opening, a transparent region of the first wall piece 2110, a magnetic portion, a reflective region of the outer face, a colored region of the outer face 2114, and/or at least one protrusion, depression, or combination thereof.

Referring now to FIGS. 9 and 10, a first container 2210 is positioned within the internal volume 2120. The first container 2210 includes a first container body 2212. The first container body 2212 extends between a first container proximal end portion 2220 and a first container distal end portion 2224. The first container proximal end portion 2220 defines a first container opening 2222. The first container distal end portion 2224 includes a first container neck 2226. The first container neck 2226 is occluded by a first frangible seal 2214. The first frangible seal 2214 is coupled to the first container neck 2226 via a first aluminum crimp 2228. In some embodiments, the first aluminum crimp 2228 is color-coded or otherwise marked to be indicative of the contents of the first container 2210. A first elastomeric member 2216 is disposed within the first container body 2212 to retain a portion of saline (e.g., a sodium chloride solution) within the first container 2210.

As depicted, a second container 2230 is positioned within the internal volume 2120. The second container 2230 includes a second container body 2232. The second container body 2232 extends between a second container proximal end portion 2240 and a second container distal end portion 2244. The second container proximal end portion 2240 defines a second container opening 2242. The second container distal end portion 2244 includes a second container neck 2246. The second container neck 2246 is occluded by a second frangible seal 2234. The second frangible seal 2234 is coupled to the second container neck 2246 via a second aluminum crimp 2248. In some embodiments, the second aluminum crimp 2248 is color-coded or otherwise marked to be indicative of the contents of the second container 2230. A second elastomeric member 2236 is disposed within the second container body 2232 to retain a portion of a first medicament within the second container 2230).

In some embodiments, a third container 2250 is positioned within the internal volume 2120. The third container 2250 includes a third container body 2252. The third container body 2252 extends between a third container proximal end portion 2260 and a third container distal end portion 2264. The third container proximal end portion 2260 defines a container opening 2262. The third container distal end portion 2264 includes a third container neck 2266. The third container neck 2266 is occluded by a third frangible seal 2254. The third frangible seal 2254 is coupled to the third container neck 2266 via a third aluminum crimp 2268. In some embodiments, the third aluminum crimp 2268 is color-coded or otherwise marked to be indicative of the contents of the third container 2250. A third elastomeric member 2256 is disposed within the third container body 2252 to retain a portion of a second medicament within the third container 2250. In some embodiments, the second medicament contained by the third container 2250 can be different from the first medicament contained by the second container 2030.

Referring now to FIGS. 9 and 11-14, in some embodiments, the cartridge assembly 2010 includes the manifold assembly 2300. As described herein, the manifold assembly 2300 facilitates fluidically coupling each of the cartridges to a single volume (i.e., the receiving volume) for delivery to the tube set 2400. The manifold assembly includes a manifold housing 2310, a manifold base, a set of puncturers 2320, and a set of valves. The manifold housing 2310 defines a receiving volume that has a sealed end portion and an output end portion. The receiving volume also includes an input portion through which the contents of each of the cartridges can be conveyed into the receiving volume. The output end portion is coupled to a tube 2410 of the tube set at 2400. In some embodiments, the tube 2410 can be permanently fixed (e.g., glued) to the manifold housing 2310. As described in more detail below, this arrangement produces a direction of flow, with the first container 2210 (containing saline) being coupled via the input portion at the upstream end of the receiving volume and the other cartridges being coupled via the input portion downstream from the entry of the first (i.e., saline) cartridge. In this manner, the saline flush from the first container 2210 can flow through substantially the entire receiving volume.

The plurality of puncturers 2320 can be placed in fluid communication with the receiving volume via the set of valves. The puncturers 2320 are oriented to puncture the frangible seals (e.g., the first frangible seal 2214, the second frangible seal 2234, and/or the third frangible seal 2254) occluding the distal end portions of each cartridge 2200. The puncturers 2320 can, for example, be needles or other similar structures able to convey a liquid therethrough. More specifically, the puncturers 2320 (or puncture assembly) can include a straight needle and at least one bent needle. The size and curvature of the needles within the puncturer assembly is based on the desired size and spacing of the cartridges, and also the desired volume of the receiving volume of the manifold housing 2310. As described herein, to facilitate being able to accurately deliver a series of medicament doses in a timely manner and through a lengthy tube set, it can be desirable to minimize the overall volume of the system. Accordingly, by having a smaller (i.e., shorter length) receiving volume, the manifold assembly 2300 includes different needles having various curvatures.

In some embodiments, the manifold assembly 2300 includes a set of one-way valves disposed between the puncturers 2320 and the receiving volume. The one-way valves can be an elastomeric valve that permits forward flow while preventing backflow. For example, the one-way valves can be duckbill valves in which the sealing function is an integral part of the one-piece elastomeric valve. The one-way valves default to a closed (e.g., sealed) state in the absence of a forward flow. The utilization of the one-way valves precludes the contents of any of the cartridges 2200 from being introduced to another cartridge 2200 during delivery. Said another way, the one-way valves permit a fluid flow from the corresponding puncturers 2320 only in the direction of the receiving volume and do not permit a flow from the receiving volume to any of the puncturers 2320 and on to the cartridges 2200. The one-way valves can be disposed along the input portion of the manifold housing 2310. Accordingly, the sealed end portion is upstream of the one-way valves.

The arrangement of the puncturers 2320, determines a coupling location for each cartridge 2200 relative to the manifold housing 2310. The first container 2210 is fluidically coupled to the receiving volume at coupling location, which is upstream of all other coupling locations. In so far as the first container 2210 contains the portion of saline, the portion of saline can be introduced to the receiving volume at a point that is upstream of any medicament. Accordingly, saline introduced to the receiving volume will flush any medicament within the receiving volume toward the tube set 2400 and on to the patient. Said another way, in such an orientation, a bolus of saline dispensed from the first container 2210 upstream of a dose of the first medicament and/or the second medicament ensures that the entirety of the dose of the first medicament or the second medicament is cleared from the manifold assembly 2300 and the tube set 2400 and delivered to the patient. Therefore, the introduction of the saline upstream of the medicament can ensure that the entirety of the prescribed dose is accurately delivered to the patient rather than being retained in a volume of the manifold assembly 2300 and/or the tube set 2400.

As depicted in FIGS. 11 and 14A, prior to the actuation of the cartridge assembly 2010, a separation distance SD is maintained between the puncturers 2320 and the plurality of cartridges 2200. In other words, the proximal end of each of the puncturers 2320 is separated from a corresponding axially-aligned frangible seal (e.g., the first frangible seal 2214, the second frangible seal 2234, and/or the third frangible seal 2254) prior to the actuation of the cartridge assembly 2010. However, as depicted in FIGS. 13 and 14B, when the cartridge assembly 2010 is in an actuated state, the plurality of cartridges 2200 are fluidically coupled to the manifold assembly 2300 via the plurality of puncturers 2320. In the actuated state, the proximal end of each of the puncturers 2320 penetrates the corresponding frangible seal and enters a volume defined by container body of the corresponding cartridge 2200. In other words, the first container 2210 and the second container 2230 are fluidically coupled to the receiving volume of the manifold assembly 2300 via the plurality of puncturers 2320 when the cartridge assembly 2010 is in the actuated state. In some embodiments, the first container 2210, the second container 2230, and the third container 2250 are fluidically coupled to the receiving volume of the manifold assembly 2300 via the plurality of puncturers 2320 when the cartridge assembly 2010 is in the actuated state. In other embodiments, the puncturers 2320 and/or cartridges can be moved sequentially so that one of the cartridges is fluidically coupled to the receiving volume while another of the cartridges remains fluidically isolated from the receiving volume. Similarly stated, in some embodiments, the cartridge assembly 2010 can have multiple different actuated states, each actuation state corresponding to a different cartridge being fluidically coupled to the receiving volume.

Referring now to FIGS. 11-14C, the housing 2100 defines the longitudinal axis A_{L}. The longitudinal axis A_{L} extends along a long axis of the housing 2100 in the proximal direction and in the distal direction. In some embodiments, the longitudinal axis A_{L} corresponds to a central axis of the cartridge assembly 2010. The manifold assembly 2300 is at the fixed longitudinal position LP_{F} when the cartridge assembly 2010 is in a stored state, as depicted in FIG. 11. The manifold assembly 2300 is at the fixed longitudinal position LP_{F} when the cartridge assembly 2010 is in the first (i.e., loaded) configuration (FIGS. 12 and 14A) and in the actuated state, as depicted in FIGS. 13 and 14B-C. Said another way, the manifold assembly 2300 is at the same fixed longitudinal position LP_{F} when the cartridge assembly 2010 is in both the stored state Ss and the actuated state S_{A}. With this arrangement, the cartridges (and not the manifold assembly) move within the housing 2100, as described below, to transition the cartridge assembly 2010 from the stored state (or a first configuration, in which the cartridge assembly 2010 is within the pump assembly 2020 but is not actuated) to the actuated state (or a second configuration, in which the cartridge assembly is both coupled to the pump assembly 2020 and one or more cartridges is fluidically coupled to the manifold assembly 2300).

In some embodiments, the plurality of cartridges 2200 are movable within the internal volume 2120. As such, the plurality of cartridges 2200 are at a first longitudinal position LP₁ when the cartridge assembly 2010 is in the stored state. The plurality of cartridges 2200 are at a second longitudinal position LP₂ when the cartridge assembly 2010 is in the actuated state. The second longitudinal position LP₂ is distal to the first longitudinal position LP₁. Said another way, the first container 2210, the second container 2230, and the third container 2250 are movable within the internal volume 2120. The first container 2210, the second container 2230, and the third container 2250 are at the first longitudinal position LP₁ when the cartridge assembly 2010 is in the stored state and at the second longitudinal position LP₂ that is distal to the first longitudinal position LP₁ when the cartridge assembly 2010 is in the actuated state.

As depicted in FIGS. 11 and 13, the gate member 2500 is positioned within the internal volume 2120. When in a locked position P_{L} (e.g., a locked orientation, an extended orientation, an initial orientation, or a design position), the gate member 2500 maintains the separation distance SD between the plurality of puncturers 2320 and the frangible seals of the plurality of cartridges 2200, as described in more detail below. The gate member 2500 is in the locked position P_{L}, when the cartridge assembly 2010 is in the stored state.

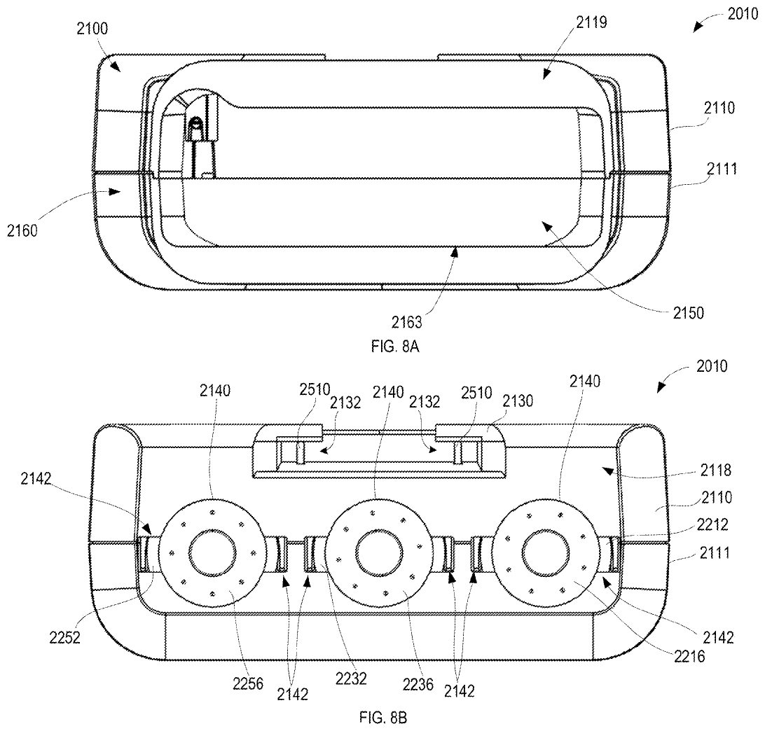

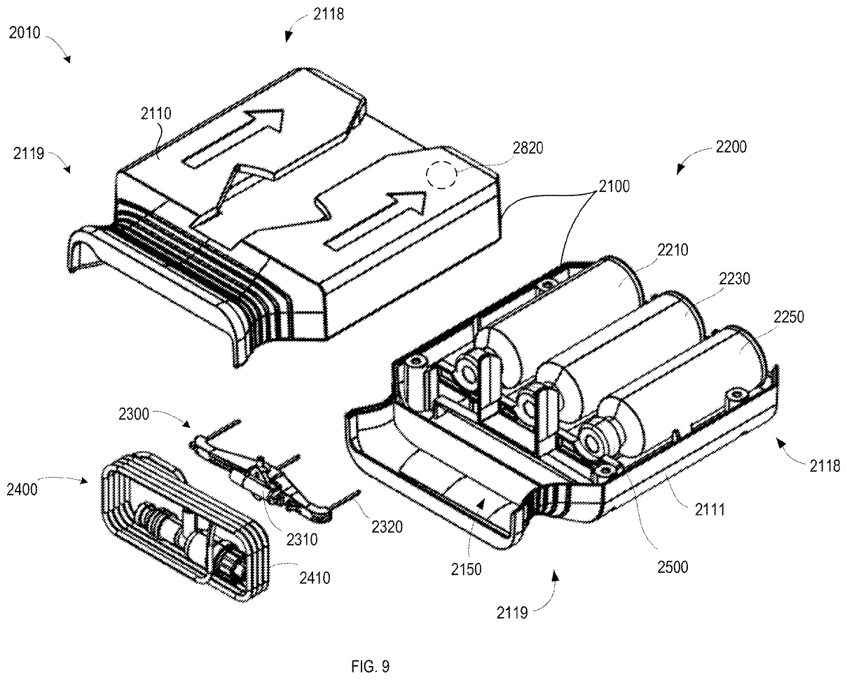

In the lock position P_{L}, the gate member 2500 has an actuation portion 2510 that is at a position between the outer face 2114 and the coupling member 2130 as depicted in FIGS. 7 and 8B. In other words, the actuation portion 2510 extends from the internal volume 2120 through the first wall piece 2110. The actuation portion 2510 is positioned such that when the cartridge assembly 2010 is coupled to the pump assembly 2020, the actuation portion 2510 is engaged by a coupling protrusion 2652 of the coupling interface 2650 of the pump assembly 2020. The engagement of the actuation portion 2510 by the coupling protrusion 2652 transitions the gate member 2500) from the locked position P_{L} to an unlocked position P_{U}. For example, as depicted in FIGS. 12 and 13, the transition to the unlocked position P_{U} corresponds to a movement of the gate member 2500 toward the second wall piece 2111 (e.g., a movement away from the coupling interface 2650). Although the actuation portion 2510 is shown as including two protrusions that extend from the internal volume 2120, in other embodiments, an actuation portion can include any structure that extends from the housing 2100 to be actuated when the cartridge assembly 2010 is coupled to the pump assembly 2020.

In some embodiments, the actuation portion 2510 extends from within the internal volume 2120 to the position between the outer face 2114 and the coupling member 2130. Said another way, in some embodiments, the actuation portion 2510 of the gate member 2500 is positioned within the keyway 2132 as depicted in FIGS. 7 and 8B. In some embodiments, the gate member 2500 includes more than one actuation portion 2510. For example, as depicted in FIG. 8B, the gate member 2500 includes two actuation portions 2510 that extend perpendicularly from the engagement member 2520 in parallel. In some embodiments wherein the gate member 2500 is a unitary structure, the actuation portion 2510 is formed during the manufacture of the gate member 2500 from the same material.

In some embodiments, a portion of the gate member 2500 is oriented to engage the first container 2210, the second container 2230, and the third container 2250. When the gate member 2500 is in the locked position P_{L}, the portion limits a movement of the plurality of cartridges 2200 from the first longitudinal position LP₁ in a distal direction. Said another way, the interaction of the proximal face of gate member 2500 with the first aluminum crimp 2228, the second aluminum crimp 2248, and/or the third aluminum crimp 2268 precludes movement of the first container 2210, the second container 2230, and/or the third container 2250 from the first longitudinal position LP₁ to the second longitudinal position LP₂. Similarly stated, when the gate member 2500 is in the locked position P_{L}, the portion of the gate member is between the proximal-most tip of the puncturers 2320 and the distal most portion of the aluminum crimps of the cartridges.

As depicted in FIGS. 1-8, the pump assembly 2020 and the cartridge assembly 2010 are configured to be coupled via an engagement between the coupling member 2130 of the cartridge assembly 2010 and the coupling interface 2650 of the pump assembly 2020. Accordingly, the coupling interface 2650 includes a coupling protrusion 2652. The coupling protrusion 2652 can be aligned parallel to the longitudinal axis A_{L}. The coupling protrusion 2652 can be formed as a T-track, as depicted in FIG. 5. The coupling protrusion 2652 can be integrally formed with the wall 2602 of the pump housing 2600. However, in some embodiments, the coupling protrusion 2652 can be coupled to the wall 2602 via fasteners, welding, an adhesive, and/or other suitable means.

In some embodiments, the pump housing 2600 as a ratio of a lateral length L_{LA} (e.g., width) to a longitudinal length L_{LO} that is between 1:2.40 and 1:2.60. For example, in some embodiments the longitudinal length L_{LO} of the housing can be 250 mm, while the lateral length L_{LA} is 100 mm. With such a ratio, the pump housing 2600 is sized to be retained in a single hand of an operator. This one-handed support of the system 2000 ensures portability of the system 2000 and facilitates the response to the acute medical condition. The portability of the system 2000 is desirable and that it facilitates treatment of the patient at the patient's location rather than requiring the patient.

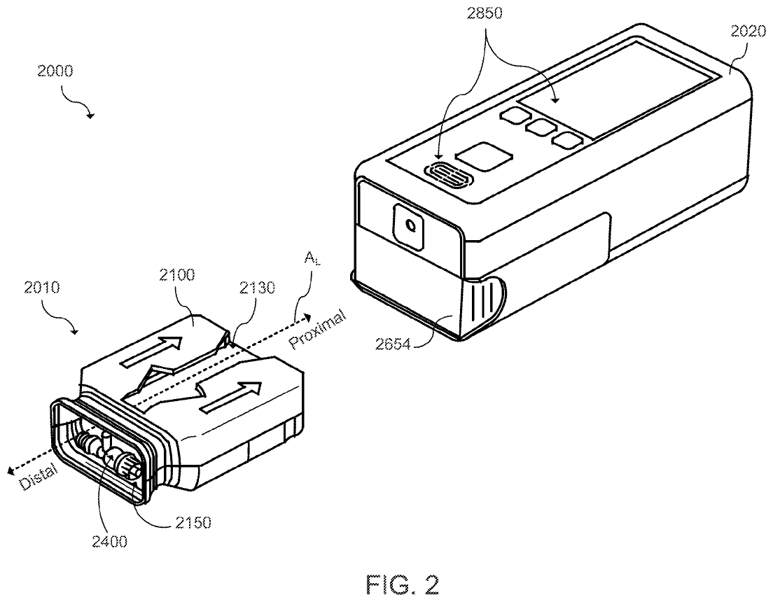

As depicted in FIGS. 1-6, in some embodiments the coupling interface 2650 includes a recess formed by a portion of the wall 2602. In such embodiments, the coupling interface 2650 is configured to receive and at least partially surround the cartridge assembly 2010. The portion of the wall 2602 that defines the recess can eliminate or mitigate potential pinch points between the cartridge assembly 2010 and the pump housing 2600 during coupling of the cartridge assembly 2010 to the pump assembly 2020. As depicted in FIG. 2, in some embodiments, the pump assembly 2020 includes a removable cap member 2654 that is sized to selectively occlude the coupling interface 2650 when the pump assembly 2020 is in a stored state. The cap member 2654 can be formed with instructions and/or surface features to facilitate removal of the cap member 2654 from the coupling interface 2650 prior to the insertion of the cartridge assembly 2010. Insofar as the cap member 2654 includes the coupling interface 2651 installed, the cap member 2654 prevents intrusion of foreign objects into the recess when the pump assembly 2020 is in a stored state. In some embodiments, the cap member 2654 can include a retention member that is fixedly coupled to the pump housing 2600 to prevent loss of the cap member 2654 when from the coupling interface 2651.

The coupling interface 2650 can, as depicted in FIG. 5, include an inspection port 2610. Inspection port 2610 can be an opening defined by the pump housing 2600. In some embodiments, inspection port 2610 and be occluded by a transparent element (e.g., window) or cover to prevent intrusion of foreign objects into the recess when the pump assembly 2020 is stored. The inspection port 2610 can be aligned with a status opening (not shown) (e.g., window) of the cartridge assembly 2010. When aligned, the inspection port 2610 and the status opening can be utilized by a user of the system 2000 to verify the contents of the cartridge assembly 2010 before or after employment.

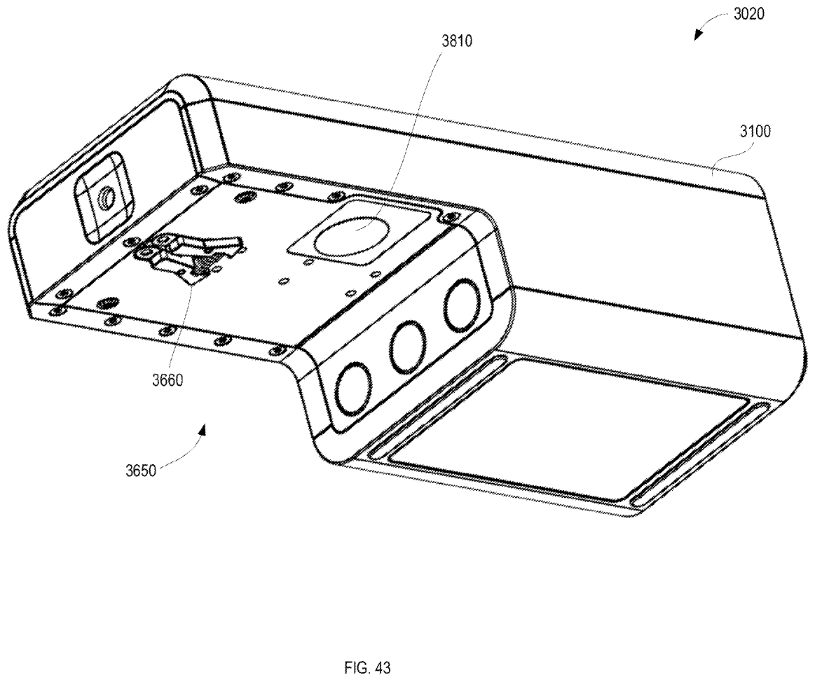

As depicted in FIGS. 5 and 6, the coupling interface 2650 includes a latch member 2660 (See e.g., latch member 3660 in FIG. 43). When the cartridge assembly 2010 is coupled to the pump assembly 2020, the latch member 2660 transitions to a lock position and engages the receiver structure 2134 of the cartridge assembly 2010. For example, the latch member 2660 can be aligned with the coupling protrusion 2652 when in an unlock position and may rotate laterally to the lock position upon actuation of the pump assembly 2020. In other embodiments, the latch member 2660 can be recessed within the pump housing 2600 in the lock position and can extend from the wall 2602 into the coupling interface 2650 in the lock position. In some embodiments, the latch member 2660 can be biased toward the lock position by a biasing element, such as a spring, a gear, a weight, and/or an elastomeric member. When in the lock position, the engagement between the latch member 2660 and the receiver structure 2134 limits movement of the cartridge assembly 2010 relative to the pump assembly 2020 in a direction parallel to the longitudinal axis A_{L} of the system 2000 (FIG. 1). In this manner, the axial position of the cartridge assembly 2010 can be fixed relative to the pump assembly 2020 during use, which enhances the accuracy of the dose delivered.

Referring now to FIGS. 15-20, in some embodiments, the drive assembly 2700 includes a plurality of drive members (e.g., the first drive member 2710, the second drive member 2730, and/or a third drive member 2750), a plurality of motors, and a plurality of lead screws all supported by a drive assembly chassis 2702. Accordingly, the drive assembly 2700 is configured to produce delivery forces F_{D} to convey doses of medicament and saline from the cartridge assembly 2010. For example, upon receiving a command from the controller 2802, a motor of the plurality of motors may cause a corresponding lead screw of the plurality of lead screws to rotate to move a corresponding drive member longitudinally in a distal direction to exert the delivery force F_{D} on an axially aligned elastomeric member to dispense a dose of the contents of the corresponding container via the tube set 2400 of the cartridge assembly 2010.

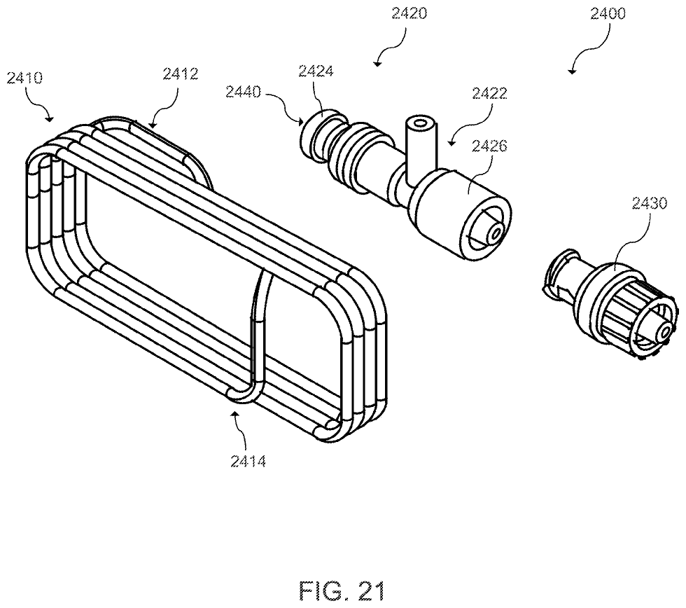

The drive assembly chassis 2702 is a rigid structure formed to support, position, and align the various components of the drive assembly 2700 discussed herein. The drive assembly chassis 2702 can, in some embodiments, be monolithically formed as a unitary component. However, in some embodiments, drive assembly chassis 2702 can be assembled by coupling together separately formed sub elements. The drive assembly chassis 2702 defines a plurality of guide slots 2704 (FIG. 18). The plurality of guide slots 2704 extend parallel to the longitudinal axis A_{L} of the pump assembly 2020.

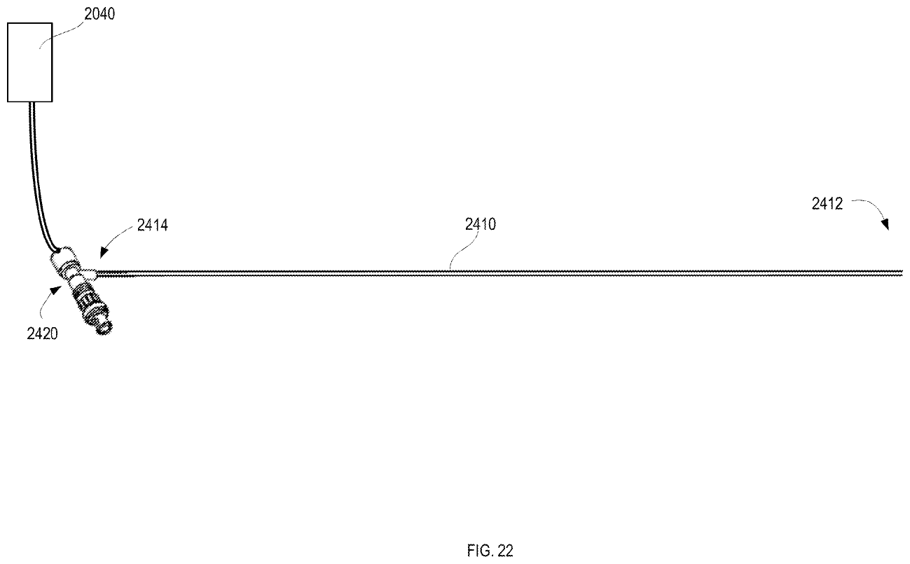

The plurality of guide slots 2704 establish, at least in part, a path of travel and/or a range of motion of the drive members. Accordingly, each of the first drive member 2710, the second drive member 2730, and the third drive member 2750 includes a piston guide block 2770 (FIG. 19). Each piston guide block 2770 can be integrally formed with each of the drive members or coupled thereto. The piston guide block 2770 is sized and oriented to be received by one of the plurality of guide slots 2704 of the drive assembly chassis 2702. The piston guide block 2770 is configured to guide the longitudinal travel of the drive members in the distal direction and in the proximal direction while maintaining a design alignment of the drive members. Additionally, the piston guide block 2770 can include elements that are sensed by a longitudinal position sensor(s) 2830 coupled to the drive assembly chassis 2702. For example, each piston guide block 2770 can include a magnetic element that is detectable by a hall-effect sensor positioned adjacent to each guide slot 2704. In such an embodiment, the interaction of the piston guide block 2770 and the longitudinal position sensor 2830 provides an indication of a longitudinal position of the corresponding drive member.

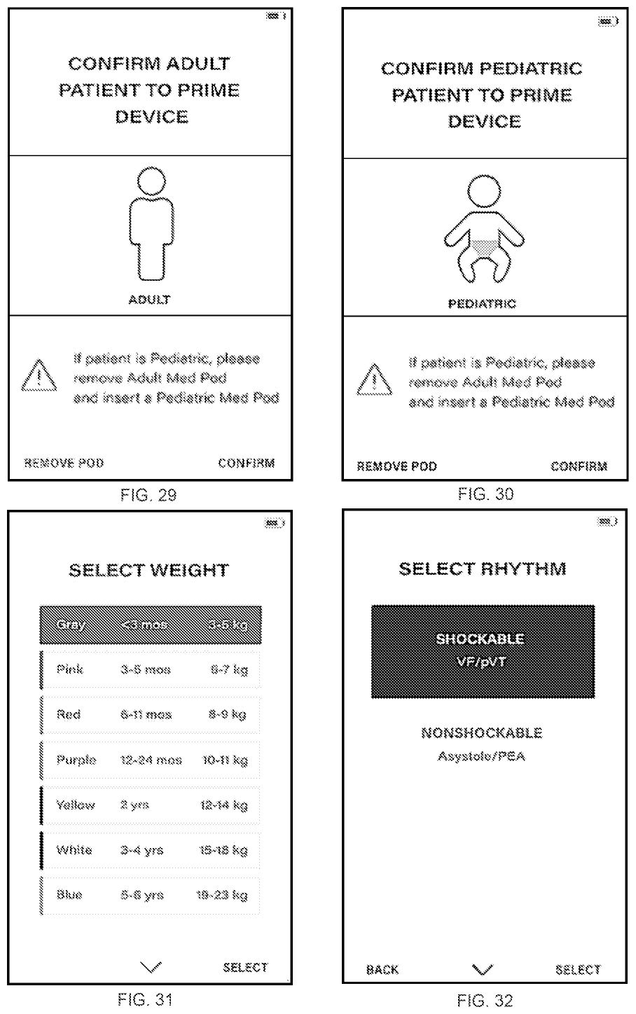

As depicted in FIGS. 15-17, in some embodiments the drive assembly 2700 includes a plurality of motors, such as a first motor 2711, a second motor 2731, and/or a third motor 2751. As depicted in FIG. 23, each of the motors is operably coupled to the controller 2802 and to a power supply 2801 (e.g., a battery). The plurality of motors can include a plurality of stepper motors, pneumatic motors, or other similar motors. Each of the motors is actuatable independently of each other motor of the plurality of motors. Each of motor of the plurality of motors defines a motor axis A_{M}. The motor axis A_{M} for each motor is parallel to the longitudinal axis A_{L} of the pump assembly 2020. In some embodiments, the plurality of motors is arranged to be coplanar in a single plane defined by the motor axis A_{M} for each motor. In some embodiments, the second motor 2731 is positioned equidistant between the first motor 2711 and the third motor 2751. Said another way, the plurality of motors can be distributed across a lateral width of the pump assembly 2020 within the pump housing 2600.

In some embodiments, each of the motors is operably coupled to one of a plurality of lead screws. For example, the first motor 2711 can be operably coupled to a first lead screw 2712 via a set of gears supported by a proximal portion of the drive assembly chassis 2702. Similarly, the second motor 2731 can be operably coupled to a second lead screw 2732 via a set of gears supported by the proximal portion of the drive assembly chassis 2702. Additionally, the third motor 2751 can be operably coupled to a third lead screw 2752 via a set of gears supported by the proximal portion of the drive assembly chassis 2702. Each lead screw defines a lead-screw axis A_{LS}. The lead-screw axis A_{LS} for each lead screw is parallel to the longitudinal axis A_{L} of the pump assembly 2020. The lead-screw axis A_{LS} for each lead screw is parallel to and displaced from the motor axis A_{M} for each motor of the plurality of motors. Additionally, each guide slot of the plurality of guide slots 2704 is displaced from In some embodiments, the plurality of lead screws is arranged to be coplanar in a single plane defined by the lead-screw axis A_{LS} for each lead screw. In some embodiments, the second lead screw 2732 is positioned equidistant between the first lead screw 2712 and the third lead screw 2752. Said another way, the plurality of lead screws can be distributed across the lateral width of the pump assembly 2020 within the pump housing 2600. Each lead screw can be coupled to a corresponding drive member via a drive nut 2780. The drive nut 2780 being configured to translate a rotational motion of the drive screw into a longitudinal motion of the drive member coupled thereto.

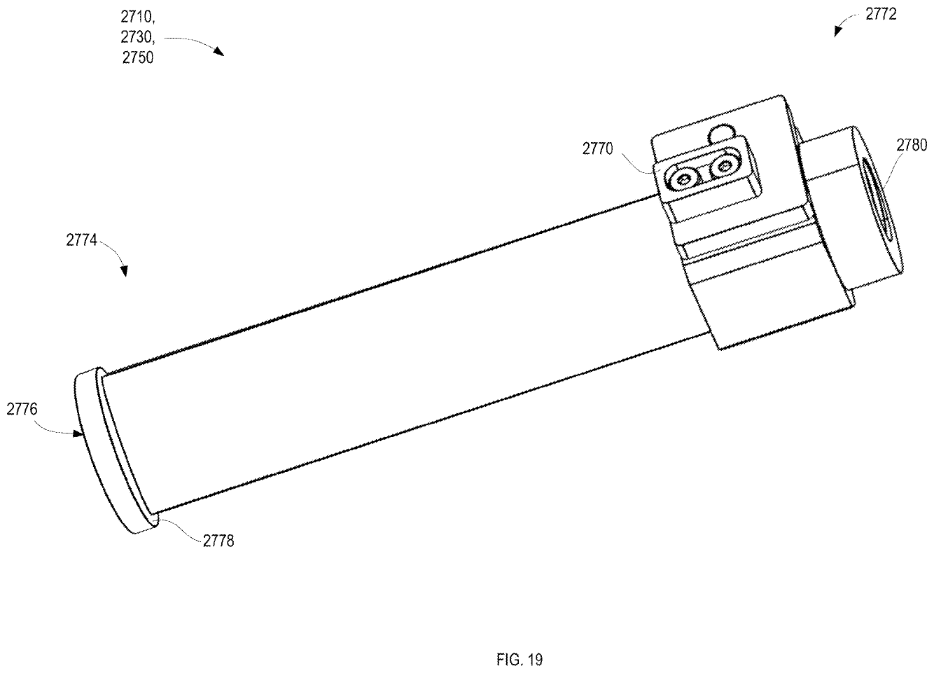

As depicted in FIG. 16, in some embodiments, a load cell assembly 2840 is coupled to a proximal end portion of the drive assembly chassis 2702. The load cell assembly 2840 can include a plurality of sensing elements positioned to measure an axial load experienced by each of the lead screws in response to a rotational input from the corresponding motor. For example, under design operations, the load placed on the lead screw corresponds to a force magnitude required to dispense a dose from the cartridge 2200. However, deviations from the design operations (e.g., a clog, a jam, or a previously used cartridge 2200) can result in an increased load and can be indicative of a fault condition. The increased load can correspond to an increased axial load in the proximal direction.

As depicted in FIG. 19, in some embodiments each drive member of the plurality of drive members is configured as a piston. Each drive member has a generally hollow cylindrical body extending between an open proximal end portion 2772 and a distal end portion 2774. The proximal end portion can surround and secure the drive nut 2780. The distal end portion 2774 can include a contact face 2776 configured to engage an elastomeric member of the cartridges 2200 in order to exert the delivery force F_{D} on the elastomeric member. For example, in some embodiments, the second drive member 2730 can be a second piston configured to exert the second delivery force F_{D} on a second elastomeric member 236 of the second container 2230 to convey a dose of medicament. Similarly, the first drive member 2710 can be a first piston configured to exert the first delivery force on a first elastomeric member 2216 of the first container 2210 to convey a dose of saline after the conveying of the dose of medicament.

Referring now to FIGS. 12, 14, 15, 19, and 20, in some embodiments the drive assembly 2700 includes a set of actuation members, such as a first actuation member 2724, a second actuation member 2744, and/or a third actuation member 2764. Each actuation member circumscribes a corresponding drive member and is coaxial with the drive member in a nested arrangement. Each actuation member can have an inner diameter that is greater than an outer diameter of the cylindrical body of the corresponding drive member but less than a maximal diameter of a retention portion 2778. Accordingly, the first actuation member 2724 can circumscribe the first drive member 2710, the second actuation member 2744 can circumscribe the second drive member 2730, and the third actuation member 2764 can circumscribe the third drive member 2750.

Each actuation member includes at least one engagement lug 2706 with a diameter that is greater than a maximal diameter of the contact face 2776. The engagement lugs 2706 are positioned to be received by one of the plurality of actuation slots 2142 (FIGS. 7 and 8B) of the cartridge assembly 2010. Accordingly, the engagement lugs 2706 are positioned to exert an actuation force F_{A}, as depicted in FIG. 14B, on the container body of each cartridge 2200 in response to the pump assembly 2020 being actuated. The actuation force F_{A} causes the cartridge 2202 to move longitudinally in a distal direction. This longitudinal movement causes the plurality of puncturers 2320 to penetrate the corresponding axially aligned frangible seals of the cartridges 2200, thereby placing each cartridge 2200 in fluid communication with the manifold assembly 2300. For example, the engagement lug(s) 2706 of the first actuation member 2724 is positioned to exert a first actuation force F_{A} on the first container body 2212 to cause the first container 2210 to be placed in fluid communication with the manifold assembly 2300 via one of the puncturers 2320. Similarly, the engagement lug(s) 2706 of the second actuation member 2744 is positioned to exert a second actuation force F_{A} on the second container body 2232 to cause the second container 2230 to be placed in fluid communication with the manifold assembly 2300 via one of the puncturers 2320. Additionally, the engagement lug(s) 2706 of the third actuation member 2764 is positioned to exert a third actuation force F_{A} on the third container body 2252 to cause the third container 2250 to be placed in fluid communication with manifold assembly 2300 via one of the puncturers 2320.

In some embodiments, such as depicted in FIGS. 14A-14C, an energy storage device 2726 is positioned between the wall 2602 of the pump housing 2600 and each actuation member (e.g., the first actuation member 2724, the second actuation member 2744, and/or the third actuation member 2764). For example, the energy storage devices 2726 can be positioned at least partially within a corresponding recess defined by the wall 2602 of the pump housing 2600. The energy storage devices 2726 are configured to generate the actuation force F_{A} in a distal direction when actuated. In some embodiments, as depicted in FIG. 14A, each energy storage devices 2726 can be a spring or elastomeric member that has a compressed state prior to actuation and, as depicted in FIGS. 14B and 14C, an extended state when actuated.

In some embodiments, the energy storage device 2726 at least partially circumscribes the corresponding drive member. For example, a first energy storage device 2726 can surround a portion of the first drive member 2710, a second energy storage device 2726 can surround a portion of the second drive member 2730, and a third energy storage device 2726 can surround a portion of the third drive member 2750. Said another way, each energy storage device 2726 can be a spring that is positioned to be coaxial with a corresponding drive member.

As depicted in FIGS. 14 and 19, each drive member includes a retention portion 2778. The retention portion thousand 2778 can extend radially outward from the cylindrical body of the drive member to a maximal diameter. The retention portion 2778 can be a lip, a ledge, a protrusion, or other similar structure configured to maintain the actuation member at an initial longitudinal position, as depicted in FIG. 14A, when the drive member is in a ready state. As such, the retention portion 2778 is positioned distal to at least a portion of the corresponding actuation member. The engagement of the actuation member by the retention portion 2778 establishes a separation distance SD between the engagement lug 2706 and the aligned container body. For example, the first drive member 2710 can include a first retention portion 2778 positioned to engage the first actuation member 2724 when the first drive member 2710 is in the first (i.e., loaded) configuration (as depicted in FIG. 14A) to establish the separation distance SD between the engagement lug(s) 2706 of the first actuation member 2724 and the first container body 2212.

As depicted in FIG. 14B, when actuated, the drive assembly 2700 is configured to move each drive member in the distal direction. This longitudinal movement of the drive member, and the retention portion 2778 thereof, facilitates a corresponding longitudinal movement in the distal direction of each actuation member. The longitudinal movement of the drive member positions the contact face 2776 within the corresponding container opening and in contact with the corresponding elastomeric member. The longitudinal movement of the drive member also facilitates placing the actuation member in contact with the corresponding container body. When in contact with the container body, the engagement lug(s) 2706 (FIG. 12) of the actuation member exerts the actuation force F_{A} on the container body causing the cartridge 2200 to transition to the second longitudinal position LP₂. Said another way, the first drive member 2710 can be configured to disengage from the first actuation member 2724 and exert a first delivery force F_{D} on the first elastomeric member 2216 of the first container 2210 when in an actuated state. In conjunction therewith, the engagement lug(s) 2706 of the first actuation member 2724 is configured to exert the first actuation force F_{A} on the first container body 2212 when in the actuated state. Similarly, the second drive member 2730 can be configured to disengage from the second actuation member 2744 and exert a second delivery force F_{D} on the second elastomeric member 2236 of the second container 2230 when in an actuated state. In conjunction therewith, the engagement lug(s) 2706 of the second actuation member 2744 is configured to exert the second actuation force F_{A} on the second container body 2232 when in the actuated state. Additionally, the third drive member 2750 can be configured to disengage from the third actuation member 2764 and exert a third delivery force F_{D} on the third elastomeric member 2256 of the third container 2250 when in an actuated state. In conjunction therewith, the engagement lug(s) 2706 of the third actuation member 2764 is configured to exert the third actuation force F_{A} on the third container body 2252 when in the actuated state.

As depicted in FIG. 14C, in some embodiments, each energy storage device 2726 is positioned to bias a corresponding container body against the manifold assembly 2300 when in the actuated state. In other words, even after the actuation force F_{A} places the cartridges 2200 in fluid communication with the manifold assembly 2300, the actuation force F_{A} is maintained throughout the employment of the system 2000. It should be appreciated that maintaining the actuation force F_{A} fixes the cartridges 2200 at the second longitudinal position LP₂ and, thus, facilitates the accurate conveying of doses from the cartridges 2200.

As described herein, when fluidically coupled to the manifold assembly 2300, the cartridges 2200 are operably coupled to a medical port (not shown) via the tube set 2400. When the cartridge assembly 2010 is in the stored state, the tube set 2400 is contained within a coil recess 2150 (see e.g., FIG. 3) defined by the housing 2100. As depicted in FIG. 8A, the housing 2100 defines an opening 2163 to the coil recess 2150 at the distal end portion 2119 of the housing 2100. In some embodiments, the opening 2163 can be occluded by a removable cover (not shown). For example, the removable cover can be a peel-away seal that is removed in order to access the tube set 2400. In some embodiments, the removable cover can be coupled to the tube set 2400 so that removal of the removable cover can facilitate the extraction of a portion of the tube set from the coil recess 2150.

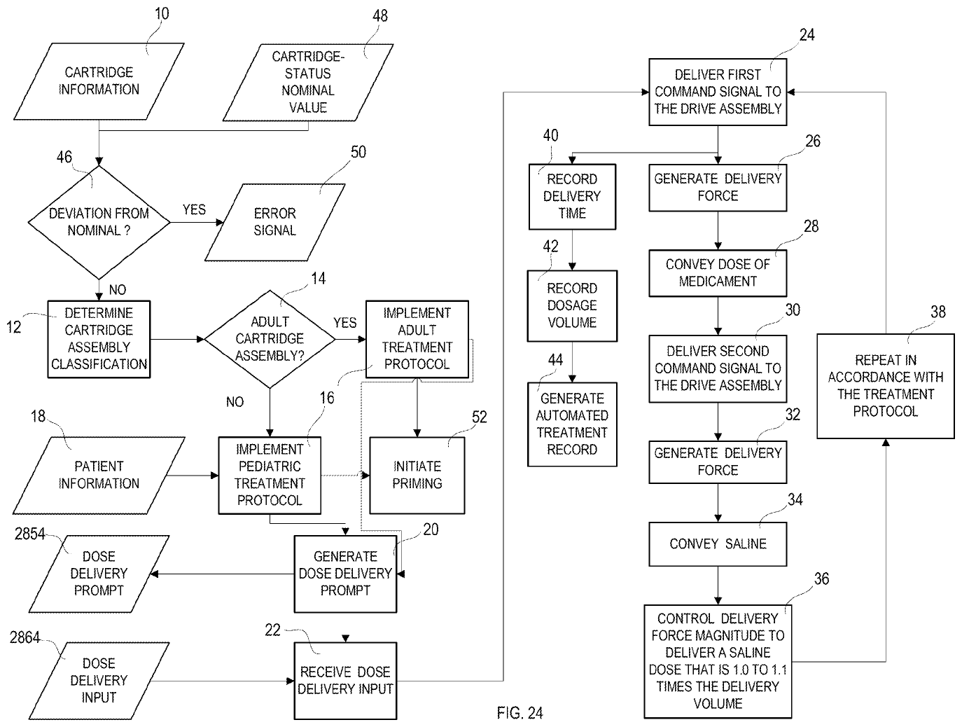

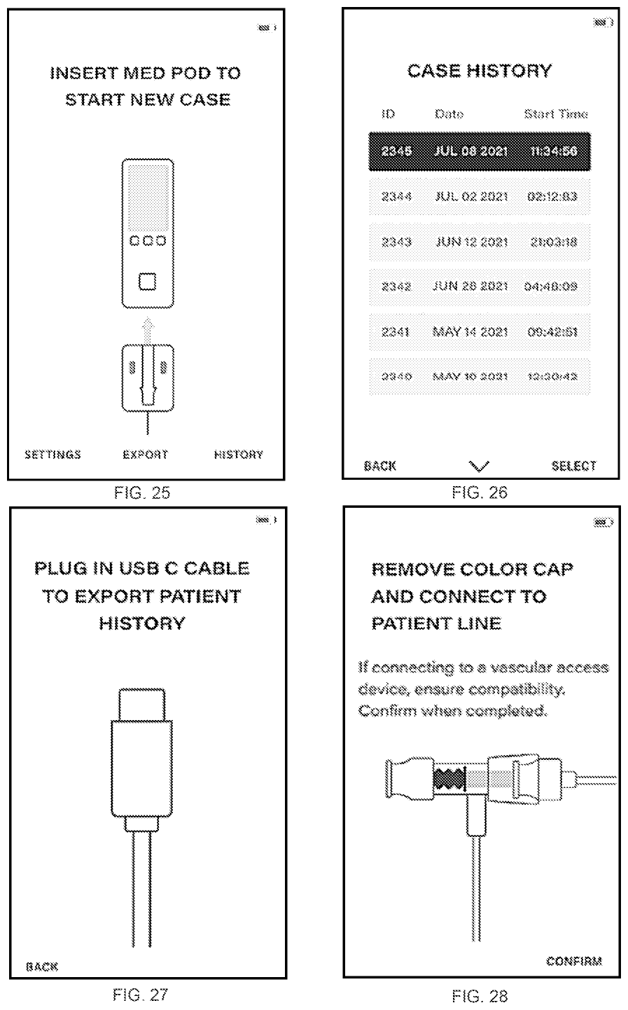

Referring to FIGS. 21 and 22, the tube set 2400 includes a tube 2410 and a T-connector 2420. The tube 2410 has a proximal tube end 2412 and a distal tube end 2414. In some embodiments, the proximal tube end 2412 is fixedly coupled to the manifold housing 2310. For example, the proximal tube end 2412 can be affixed to the manifold housing 2310 in fluid communication with the receiving volume by any suitable coupling mechanism (e.g., via an adhesive, epoxy, plastic weld, or mechanical clamp intended to be permanent).

The T-connector 2420 of the tube set 2400 includes a central connector portion 2422 extending between a proximal connector end 2424 and a distal connector end 2426. The distal tube end 2414 is fixedly coupled to the central connector portion 2422. For example, in some embodiments, the distal tube end 2414 can be coupled to the central connector portion 2422 via an adhesive, rather than via a reversible coupling member (e.g., a Luer lock or other similar structure). However, in some embodiments, the distal tube end 2414 is removably coupled to the central connector portion 2422 (such as via a Luer lock). An IV check-valve 2430 is removably coupled at the distal connector end 2426. The IV check-valve 2430 can facilitate the coupling of the cartridge assembly 2010 to the medical port (e.g., a vascular access device). The proximal connector end 2424 includes an access port 2440 (e.g., an auxiliary port). The access port 2440 can facilitate the introduction of additional medicaments and/or fluids during the response to the acute medical condition without necessitating the decoupling of the cartridge assembly 2010 from the medical port. For example, in some embodiments, a secondary saline supply 2040 can be removably coupled to the access port 2440 of the T-connector 2420. This can be accomplished while the T-connector 2420 remains coupled to the medical port and the tube 2410. The employment of the secondary saline supply 2040 can facilitate delivery of the medicament from the cartridge assembly 2010 by maintaining an open line (i.e., maintaining the patency of the medical port) during intervals between doses.

When actuated, the delivery force F_{D} exerted on each of the elastomeric members is configured to convey the dose from the corresponding cartridge 2200 to the patient via the manifold assembly 2300 and the tube set 2400. For example, the second delivery force F_{D} exerted on the second elastomeric member 2236 is configured to convey the dose of the medicament from the second container 2230 to the patient via the manifold assembly 2300 and the tube set 2400. Following the conveyance of the dose of the medicament, the first delivery force F_{D} exerted on the first elastomeric member 2216 is configured to flush the dose of the medicament from the manifold assembly 2300, the tube set 2400, and the medical port via a dose of saline drawn from the first container 2210 of the cartridge assembly 2010.

Each of the components of the cartridge assembly 2010 and the medical port through which the medicament is conveyed during delivery to a patient is characterized by a volume and a flow surface area, which can impact the accuracy and/or dose availability of the delivered medicament. Specifically, a delivery volume of the cartridge assembly 2010 is defined by a fluid volume of the manifold assembly 2300, a fluid volume of the tube set 2400, and the fluid volume of the medical port. Accordingly, in some embodiments, a magnitude and/or duration of the first delivery force F_{D} is such that the resultant dose of saline from the first container 2210 has a magnitude that is greater than or equal to 1.0 and less than or equal to 1.1 times the delivery volume of the cartridge assembly 2010. In that the volume of the dose of saline at least equals the delivery volume of the cartridge assembly, the dose of saline can ensure complete delivery of the dose of medicament to the patient by flushing the entirety of the delivery volume. Insofar as the total volume of saline available to flush the delivery volume following the conveying of a dose of medicament is established by the total volume of saline contained within the first container 2210, limiting the dose magnitude to no more than 1.1 times the delivery volume of the cartridge assembly 2010 maximizes the number of doses of saline that are available to the system 2000. This, in turn maximizes the number of doses of the medicament that can be delivered and flushed from the delivery volume.

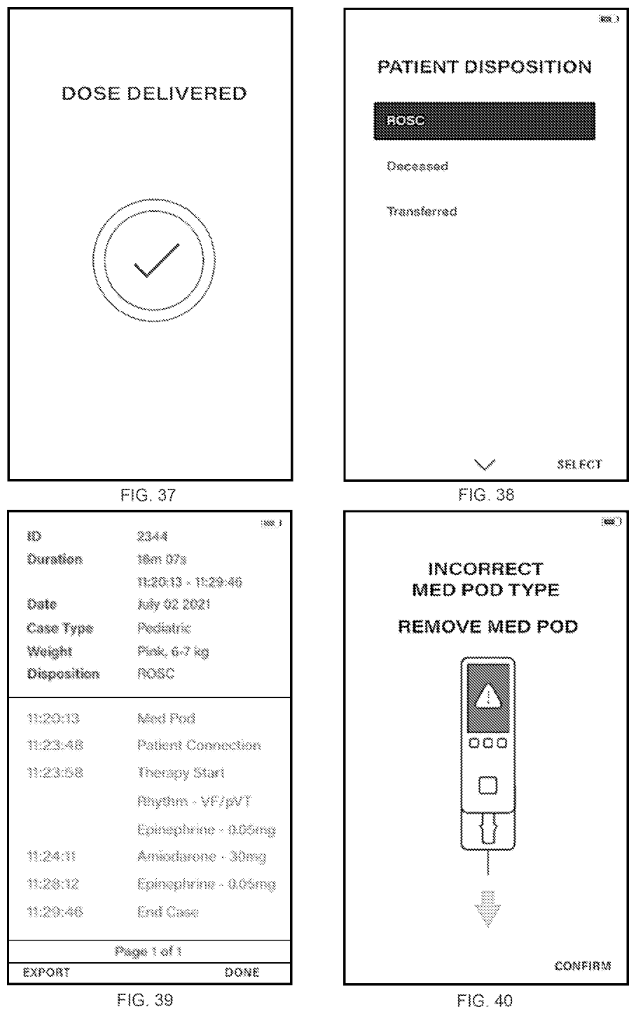

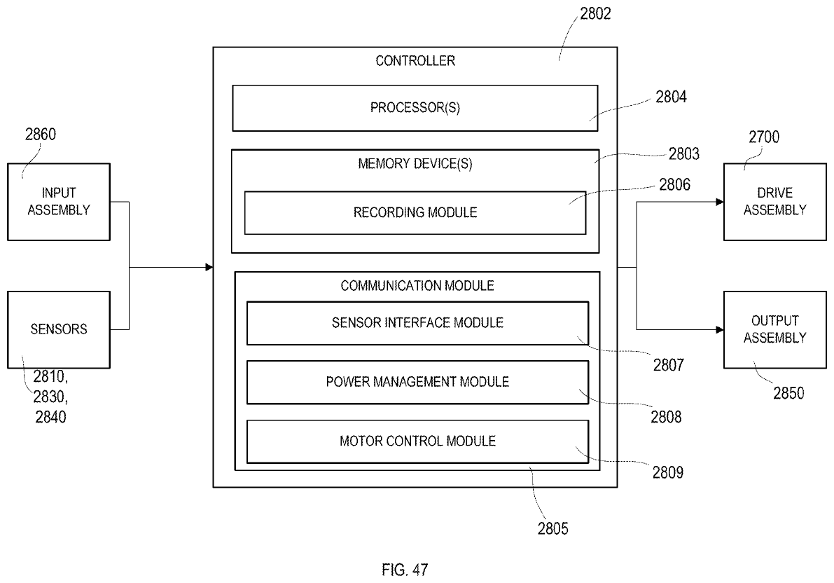

FIG. 23 is a system diagram of the system 2000. In order to automate certain aspects of a response to an acute medical condition, such as cardiac arrest, the system 2000 includes the controller 2802 that is operably coupled to an output assembly 2850, an input assembly 2860, a drive assembly 2700, and the various sensors and sensing elements described herein. Details of the controller 2802 are described below with reference to FIG. 47. The output assembly 2850 is configured to deliver a plurality of outputs 2852 to an operator of the pump assembly 2020. The output assembly 2850 can include display elements (e.g., screens and/or lights), auditory elements (e.g., speakers and/or buzzers), and/or haptic feedback elements (e.g., a vibration generator). The plurality of outputs 2852 can include audible and visual indications, such as depicted in FIGS. 25-40. The input assembly 2860 is configured to receive a plurality of inputs 2862 (e.g., button actuations) from the operator of the system 2000.

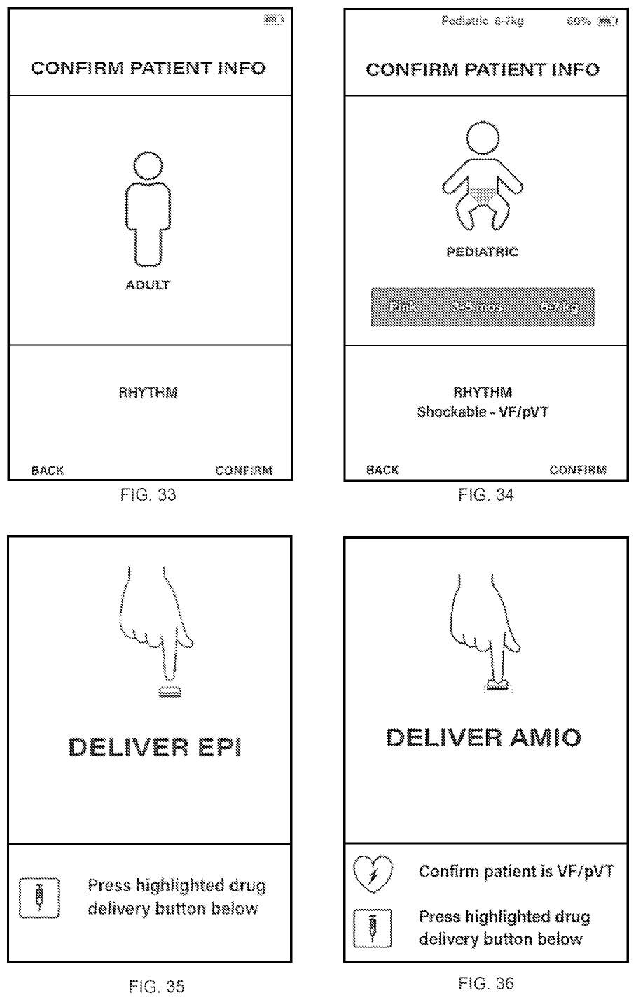

FIG. 24 is a flow chart depicting a plurality of operations of operations implemented by controller 2802 in response to an acute medical condition. As depicted, the controller 2802 receives cartridge information 10 from the sensing element 2810. At step 12, the controller 2802 determines a cartridge assembly classification of the cartridge assembly 2010 based on the input (e.g., the cartridge information 10) from the sensing element 2810. At step 14, the controller determines whether the cartridge assembly 2010 is configured to treat an adult patient. As depicted at step 16, the controller 2802 implements a treatment protocol based on the cartridge assembly classification. The treatment protocol includes at least medicament dosage information (e.g., dose magnitude and frequency) and a delivery schedule (e.g., a quantity of doses to be delivered over a specified treatment period).

For example, if the cartridge assembly 2010 is configured for the treatment of an adult patient, the implemented treatment protocol can be the Advanced Cardiac Life Support (ACLS) algorithm. Similarly, if the cartridge assembly 2010 is configured for the treatment of a pediatric patient, the implemented treatment protocol can be the Pediatric Advanced Life Support (PALS) algorithm. When the cartridge assembly 2010 is configured for the treatment of pediatric patient, patient information 18 can be provided to the controller 2802 via the input assembly 2860. The patient information can include the fix as to the age of the patient and/or the weight of the patient. The patient information 18 can be utilized to tailor the treatment protocol (e.g., FIG. 31).

As depicted at step 20, the controller 2802 generates least one output of the plurality of outputs 2852 in accordance with the treatment protocol. The output(s) corresponds to a dose delivery prompt 2854 (see e.g., FIGS. 35 and 36). In some embodiments, the dose delivery prompt 2854 is a display indication presented by the output assembly 2850. However, in some embodiments, the dose delivery prompt 2854 can be an audible prompt, a haptic prompt or a combination that includes the visual prompt. Following the generation of the dose delivery prompt 2854, the controller 2802 receives a dose-delivery input 2864 from the operator of the system 2000 at step 22. The dose-delivery input 2864 can, in some embodiments, correspond to an actuation of a dose-delivery button or combination of buttons of the input assembly 2860. In some embodiments, the dose-delivery button can include markings that indicate that actuation of the button generates the dose-delivery input 2864. In some embodiments, the dose-delivery button may be illuminated when the dose-delivery input 2864 is required in accordance with the treatment protocol.

In response to the dose-delivery input 2864, the controller 2802 delivers, at step 24. A first command signal to the drive assembly 2700. The first command signal causes the second drive member 2730 or the third drive member 2750 to exert the delivery force F_{D} (e.g., the second delivery force) on the corresponding elastomeric member the corresponding cartridge 2200. The generation of the delivery force F_{D} at step 26 conveys a dose of medicament at step 28. For example, in some embodiments the first command signal causes the second drive member 2730 to exert the second delivery force F_{D} on the second elastomeric member 2236 of the second container 2230 to convey a dose of the medicament contained within the second container 2230.

Following the conveying of the dose of medicament at step 28, the controller 2802, at step 30, delivers a second command signal to the drive assembly 2700. The second command signal causes the drive assembly 2700 to generate the second delivery force at step 32 via the first drive member 2710. The first drive member 2710 exerts the first delivery force F_{D} on the first elastomeric member 2216 of the first container 2210 to convey saline from the first container 2210 at step 34. As depicted at step 36, the first delivery force resulting from the second command signal is controlled to establish the dose of saline conveyed from the first container 2210 at a magnitude that is greater than or equal to 1.0 and less than or equal to 1.1 times the delivery volume of the cartridge assembly 2010. The conveying of the dose of saline at step 34 flushes the dose of medicament conveyed at step 28 from the cartridge assembly 2010 and ensures delivery of the complete dose of medicament to the patient.

As depicted at step 38, steps 24-36 are repeated in accordance with the treatment protocol. Accordingly, additional doses of the medicament are conveyed in accordance with the treatment protocol. Each conveyed dose of the medicament is followed by a conveyed dose of saline.

In some embodiments, the controller 2802, at step 40, can be configured to record a delivery time of each dose of the medicament. Additionally, at step 32, the controller 2802 can record a dosage volume for each dose of the medicament delivered. Further, the controller 2802 and be configured to generate an automated treatment record at step 44 (see e.g., FIG. 39). In some embodiments, the controller 2802 may utilize a recording module 2806 to record the delivery time and dosage volume for each dose of the medicament delivered.

Referring still to FIG. 24, in some embodiments, a cartridge-status input (e.g., cartridge information 10) can be received by the controller 2802 from the sensing element 2810. The cartridge-status input can include information or about the cartridge assembly 2010, such as an expiration date, a manufacture date, lot information, a storage compliance indication, a recall status, and/or a prior actuation of the cartridge. At step 46, the controller 2802 compares the cartridge-status input to a cartridge-status design value 48. When a deviation between the cartridge-status input and the cartridge-status design value 48 is detected, the controller 2802 delivers an error signal 50 to the output assembly 2850. For example, a comparison of the cartridge-status input to the cartridge-status design value 48 may indicate that the cartridge assembly 2010 is expired, is recalled, has been improperly stored, and/or has been previously actuated, and is, therefore, no longer suitable for patient treatment.

In some embodiments, the controller 2802 can deliver third command signal to the drive assembly 2700. The third command signal can, at step 52, initiate a priming sequence for the cartridge assembly 2010. In accordance with the priming sequence, each drive member and be moved a specified longitudinal distance and preparation for the generation of the delivery force F_{D}. The priming sequence may be accomplished prior to the coupling of the tube set 2402 the medical port and may convey a bolus of air from at least one of the cartridges 2200.

FIG. 25 is a screen display of the plurality of outputs 2852 of the output assembly 2850 instructing an operator of the system 2000 to couple the cartridge assembly 2010 to the pump assembly 2020.

FIG. 26 is a screen display of the plurality of outputs 2852 of the output assembly 2850 indicating a plurality of automated treatment records stored within the memory device(s) 2803 of the controller 2802.

FIG. 27 is a screen display of the plurality of outputs 2852 of the output assembly 2850 relating to the export of automated treatment records from the system 2000.

FIG. 28 is a screen display of the plurality of outputs 2852 of the output assembly 2850 providing instructions to an operator of the system 2004 coupling the tube set 2400 to the medical port.

FIG. 29 is a screen display of the plurality of outputs 2852 of the output assembly 2850 indicating that the cartridge assembly 2010 coupled to the pump assembly 2020 is configured for the treatment of an adult patient and requesting confirmation that the patient is an adult.

FIG. 30 is a screen display of the plurality of outputs 2852 of the output assembly 2850 indicating that the cartridge assembly 2010 coupled to the pump assembly 2020 is configured for the treatment of a pediatric patient and requesting confirmation that the patient is pediatric patient.

FIG. 31 is a screen display of the plurality of outputs 2852 of the output assembly 2850 requesting the selection of patient information corresponding to a pediatric patient.

FIG. 32 is a screen display of the plurality of outputs 2852 of the output assembly 2850 requesting that the operator of the system 2000 select the detected rhythm. The implementation of the treatment protocol in very depending on the detected cardiac rhythm.

FIGS. 33 and 34 are screen displays of the plurality of outputs 2852 of the output assembly 2850 that present patient information and require confirmation thereof prior to protocol implementation.

FIG. 35 is a screen display of the plurality of outputs 2852 of the output assembly 2850 indicative of a dose delivery prompt 2854 corresponding to a dose of epinephrine.

FIG. 36 is a screen display of the plurality of outputs 2852 of the output assembly 2850 indicative of a dose delivery prompt 2854 corresponding to a dose of amiodarone hydrochloride

FIG. 37 is a screen display of the plurality of outputs 2852 of the output assembly 2850 indicative of a dose delivery from the system 2000.

FIG. 38 is a screen display of the plurality of outputs 2852 of the output assembly 2850 requesting information from the operator of the system 2000 for the automated treatment record.

FIG. 39 is a screen display of the plurality of outputs 2852 of the output assembly 2850 corresponding to an automated treatment record.

FIG. 37 is a screen display of the plurality of outputs 2852 of the output assembly 2850 corresponding to an error signal.

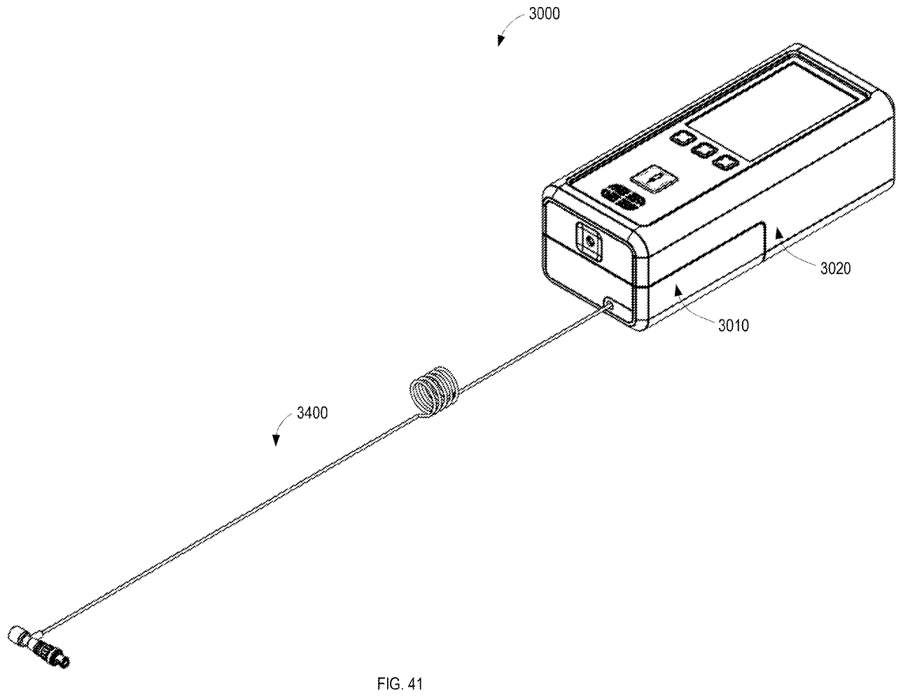

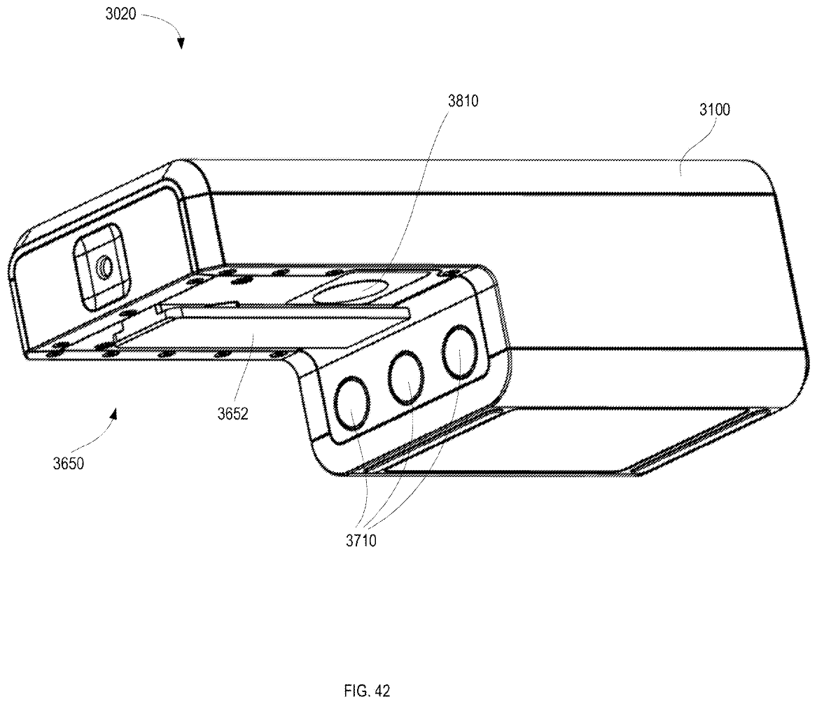

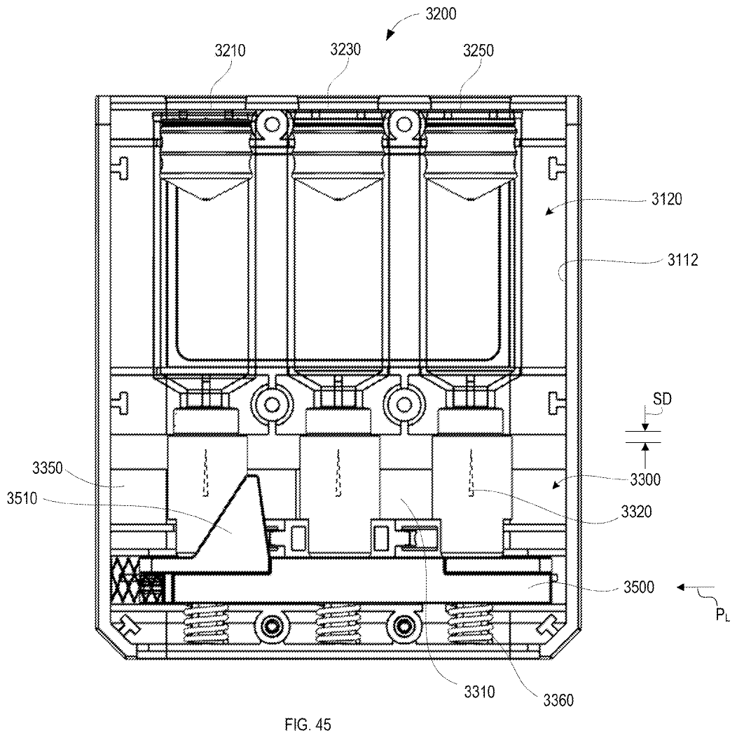

In some embodiments, a cartridge assembly can include one or more energy storage members or biasing members to facilitate actuation of the cartridge assembly after the cartridge assembly is coupled to a pump assembly. For example, FIGS. 41-46 depict various aspects of an acute medical condition response system (system) (also referred to as an Automated Medication Administration System (AMAS) or “system”) 3000. As described herein, the system 3000 is configured to automate certain aspects of a response to an acute medical condition, such as cardiac arrest. Accordingly, the system 3000 utilizes a sealed cartridge assembly 3010 containing more than one dose of a drug (e.g., epinephrine, amiodarone hydrochloride, or other desired medicament) and more than one dose of a separate saline solution. The cartridge assembly 3010 is configured to be operably coupled between a pump assembly 3020 and a medical port (not shown) that is inserted into or attached to a patient. In this manner, the system 3000 can be used with a conventional medical port. The cartridge assembly 3010 includes a housing 3100 having a wall that defines an internal volume 3120 (see FIG. 45) and includes a coupling member 3130. The coupling member 3130 is positioned to engage a coupling interface 3650 of the pump assembly 3020. A set of cartridges 3200 is positioned within the internal volume 3120. The cartridges 3200 of the cartridge assembly 3010 are substantially similar to the cartridges 2200 described above. In this embodiment, the cartridge assembly 3010 includes a first container 3210 that contains saline, a second container 3230 that contains a first medicament, and a third container 3250) that contains a second medicament. A manifold assembly 3300 is also positioned within the internal volume 3120. The manifold assembly 3300 includes a set of puncturers 3320 oriented to fluidically couple each of the cartridges 3200 to a tube set 3400 when the cartridge assembly 3010 is actuated. However, a gate member 3500 is also positioned within the internal volume 3120 to maintain a separation distance SD (see FIG. 45) between the set of puncturers 3320 and the set of cartridges 3200 when the cartridge assembly 3010 is in a storage configuration as depicted in FIG. 45. In this manner, the gate member 3500 functions as a safety to prevent the undesired or premature actuation of the cartridge assembly 3010.

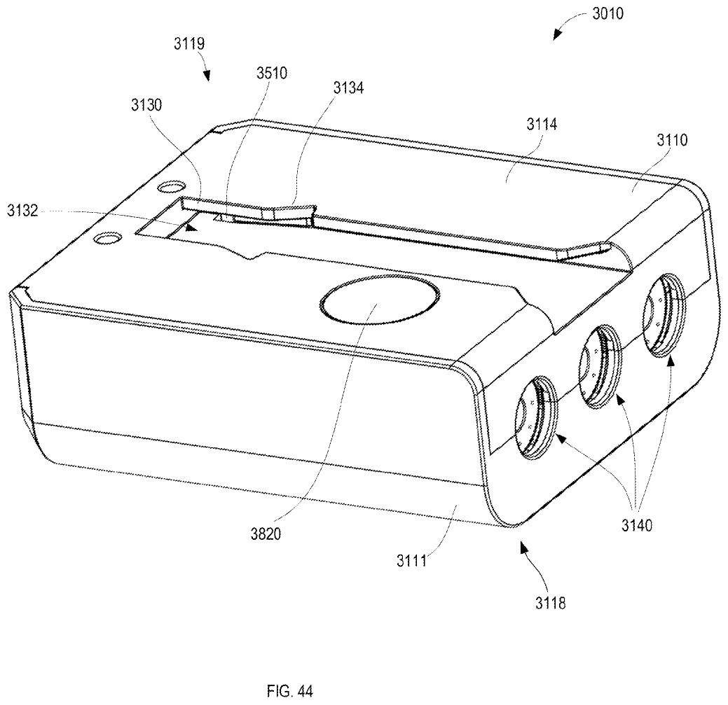

Referring to FIGS. 44-46, the cartridge assembly 3010 includes a first wall piece 3110 and a second wall piece 3111 that are coupled together to form a wall of the housing 3100. In some embodiments, the first wall piece 3110 is formed as an upper wall segment extending between a proximal portion 3118 and a distal end portion 3119 of the housing 3100. Similarly, the second wall piece 3111 can be formed as a lower wall segment extending between the proximal portion 3118 and the distal end portion 3119 of the housing 3100. The wall formed by the first wall piece 3110 and a second wall piece 3111 includes an inner face 3112. The inner face 3112 defines the internal volume 3120.

As depicted in FIG. 44 for example, an outer face 3114 of the wall (e.g., of the first wall piece 3110) includes the coupling member (or feature) 3130. The coupling member 3130 includes a keyway 3132 and a receiver structure 3134. The keyway 3132 is configured to receive a mating coupling protrusion 3652 (FIG. 27) of the pump assembly 3020 configured as a T-track. The receiver structure 3134 is configured to receive a latch member 3660 of the pump assembly 3020. Further functions and features of the coupling member 3130 are as described above with regards to the coupling member 2130.

The first wall piece 3110 and the second wall piece 3111 are formed with multiple sets of cradles. The cradles are sized and positioned to at least partially surround the plurality of cartridges 3200. As such, the plurality of cradles can align and secure the plurality of cartridges 3200 in a fixed longitudinal position. The set of cradles at the proximal portion 3118 define a portion of a boundary of a set of access orifices 3140. Thus, when the first wall piece 3110 is coupled to the second wall piece 3111, the housing 3100 defines the set of access orifices 3140 that are aligned with the elastomeric members of each cartridge 3200. In this manner, in some embodiments, a drive member 3710 of the pump assembly 3020 can exert a force on the elastomeric member of each cartridge 3200 to cause delivery of the medicament therefrom. Additionally, the housing 3100 can be formed with a status opening (not shown). The status opening can be utilized by a user of the cartridge assembly 3010 to verify the contents of the cartridge assembly 3010 before or after employment. In some embodiments, the housing 3100 can also define and/or support a sensor interface 3820 for engaging with a sensing element 3810 of the pump assembly 3020. The sensor interface 3820 can be as described above with reference to the sensor interface 2820.

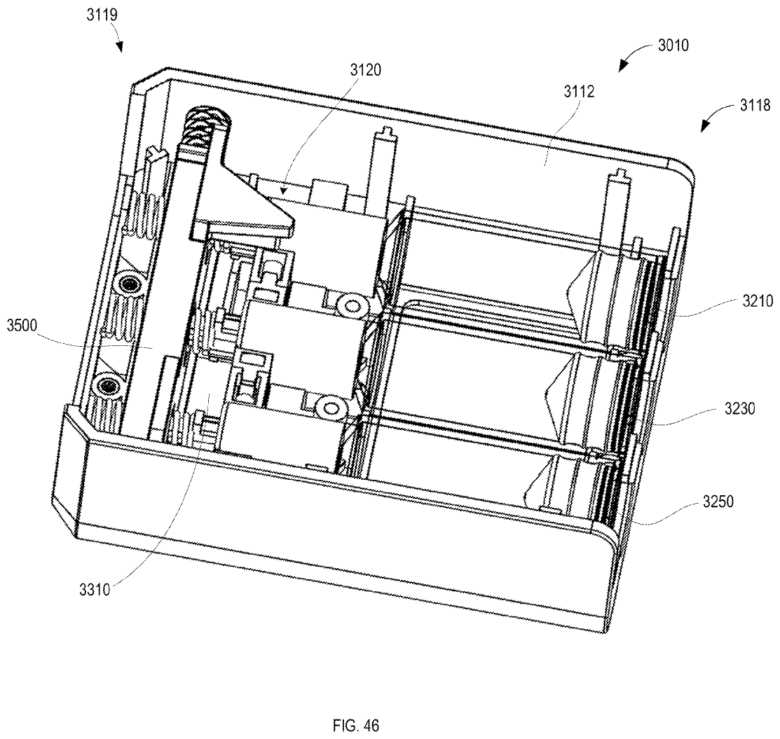

Referring to FIGS. 45-46, the manifold assembly 3300 includes a set of manifold segments 3350, a set of one-way valves (not shown), and a set of puncturers 3320. Each puncturer 3320 includes a needle circumscribed by a cylindrical housing. The one-way valves are positioned between each manifold segment 3350 and the puncturer 3320 coupled thereto. The set of manifold segments 3350 are, as depicted in FIG. 45, intercoupled to form a manifold housing 3310. The manifold housing 3310 defines a receiving volume, which is in fluid communication with the tube set 3400. The tube set 3400 is substantially similar to the tube set 2400 described above.

In some embodiments, at least one energy storage device 3360 (e.g., a spring or other biasing member) is positioned within the internal volume 3120 between the inner face 3112 of the wall and the manifold housing 3310. The energy storage device(s) 3360 is in contact with the manifold housing 3310. The energy storage device(s) 3360 is configured to exert a force on the manifold housing 3310. In response to the force and when the gate member 3500 is moved, the manifold assembly 3300 moves in a proximal direction from a stored position, as depicted in FIG. 45. Said another way, upon actuation of the cartridge assembly 3010, the manifold assembly 3300 moves in a proximal direction toward the cartridges 3200, which remain at a fixed longitudinal position relative to the housing 3100 as depicted in FIG. 46.

In some embodiments, the cartridge assembly 3010 is transitioned from a stored state to an actuated state (FIG. 46) via the coupling of the cartridge assembly 3010 to the pump assembly 3020. When the cartridge assembly 3010 is in the stored state, the separation distance SD is maintained between the puncturers 3320 and the cartridges 3200 by the gate member 3500. In other words, the proximal end of each of the needle 3322 is separated from a corresponding axially-aligned cartridge 3200 prior to the actuation of the cartridge assembly 3010. However, as depicted in FIG. 46, when the cartridge assembly 3010 is actuated, via the coupling to the pump assembly 3020, the plurality of cartridges 3200 are fluidically coupled to the receiving volume 3312 via the puncturers 3320.

As depicted in FIG. 45, the gate member 3500 is positioned within the internal volume 3120. When in a locked position P_{L} (e.g., a locked orientation, an initial orientation, or a design position), the gate member 3500 maintains the separation distance SD between the puncturers 3320 and the cartridges 3200. The gate member 2500 is in the locked position P_{L}, as depicted in FIG. 32, when the cartridge assembly 3010 is in the stored state Ss.

In the lock position P_{L}, the gate member 3500 has an actuation portion 3510 that is at a position between the outer face 3114 and the coupling member. In other words, the actuation portion 3510 extends from the internal volume 3120 through the first wall piece 3110. The actuation portion 3510 is positioned such that when the cartridge assembly 3010 is coupled to the pump assembly 3020, the actuation portion 3510 is engaged by a coupling protrusion 3652 of the coupling interface 3650 of the pump assembly 3020. The engagement of the actuation portion 3510 by the coupling protrusion 3652 results in a lateral movement of the actuation portion 3510 and thus the gate member 3500. This lateral movement transitions the gate member 3500 from the locked position P_{L}, at least temporarily, to an unlocked position, and thereby, releases the manifold assembly 3300 to respond to the force exerted by the energy storage device(s) 3360. That another way, the cartridge assembly 3010 includes a manifold assembly 3300 configured to move in a proximal direction and into fluid communication with the first container 3210, the second container 3230, and the third container 3250 in response to the gate member 3500 being moved to the unlock position as depicted in FIG. 46.

Any of the cartridge assemblies or cartridges shown and described herein can include any suitable medicament. For example, in some embodiments, a cartridge assembly (e.g., the cartridge assembly 2010 and 3010) can include one cartridge containing saline (e.g., 0.9%), one cartridge containing epinephrine and one cartridge containing amiodarone hydrochloride. In some embodiments, a cartridge assembly can include any suitable amount, concentration, and/or formulation of epinephrine, amiodarone hydrochloride, or saline. For example, in some embodiments, any of the cartridge assemblies described herein can be configured for either an adult dosage or a pediatric dosage. Table 1 below provides one example of medicaments and saline that can be included in such configurations.

TABLE 1

| Cartridge | Cartridge contents | Adult Configuration | Pediatric Configuration |
|---|---|---|---|
| 1 | Epinephrine | 1.0 mg/mL, 12.0 mL | 0.1 mg/mL 12.0 mL |
| 2 | Amiodarone HCl | 50 mg/mL, 12.0 mL | 50 mg/mL, 12.0 mL |
| 3 | 0.9% Sodium Chloride | 9 mg/mL, 12.0 mL | 9 mg/mL, 12.0 mL |

In some embodiments, any of the cartridge assemblies or any of the cartridges can contain amiodarone hydrochloride having the formulation as indicated in Table 2.

TABLE 2

| | | Amiodarone Hydrochloride Injection | |
|---|---|---|---|
| | | USP, 50 mg/mL, Single patient use | |
| Product | | cartridge | |
| Active Ingr. | Amiodarone Hydrochloride | 50 mg/mL (as the HCl) | |
| | USP | | |
| Excipients | Sulfobutylether β-cyclodextrin | 225 | mg/mL |
| | Citric Acid Monohydrate | 3.77 | mg/mL |
| | Sodium Citrate Dihydrate | 2.10 | mg/mL |

| | |
|---|---|
| Sodium Hydroxide | qs to adjust pH |
| Sodium Citrate Dihydrate | qs to adjust pH |

| Fill Volume | 12.0 | mL* |
|---|---|---|
| Dosage Form | Injection (Solution) | |
| Configuration | 600 mg/12.0 mL* in clear glass | |
| | cartridge | |
| Route of Administration | Intravenous (Bolus) | |
| Strength | 50 | mg/mL |
| Strength Concentration for Administration to | 50 | mg/mL |

| Patient | |
|---|---|
| Indication | Acute treatment of ventricular |
| | arrhythmias refractory to |
| | defibrillation, in adult and pediatric |
| | patients in cardiac arrest. |

While various embodiments have been described above, it should be understood that they have been presented by way of example only, and not limitation. Where methods and/or schematics described above indicate certain events and/or flow patterns occurring in certain order, the ordering of certain events and/or operations may be modified. While the embodiments have been particularly shown and described, it will be understood that various changes in form and details may be made.

For example, although the cartridges contained within the cartridge assemblies are shown as being the same size and having the same features, in other embodiments, any of the cartridge assemblies described herein can have one cartridge that is of a different size than the other cartridges within the cartridge assembly. For example, in some embodiments, a cartridge assembly (e.g., the cartridge assembly 2010 or 3010) can include a first container for containing saline that has a greater volume than the cartridges that contain medicaments to be delivered.

Although the cartridges assemblies are shown herein as having a cartridges containing medicaments therein, in other embodiments, a cartridge assembly can include one or more cartridges that are devoid of any medicament that can be used for training purposes. For example, in some embodiments a training cartridge assembly can include cartridges that have the color-coded crimp seals and a viewing window (as described herein), but can be devoid of any medicament. Such a training cartridge assembly can allow a user to practice coupling and decoupling a cartridge assembly to the pump assembly, as well as to simulate actual use (e.g., by actuating the pump assembly).

In some embodiments, a system can include one or more cartridge assembly blanks that are devoid of any cartridges. A cartridge assembly blank can have similar external features as any of the cartridge assemblies described herein (e.g., a coupling member similar to the coupling member 2130). The cartridge assembly blank can be coupled to the pump assembly to limit any damage to the drive portions of the pump assembly when the system is not in use.

As shown particularly in FIG. 47, a schematic diagram of one embodiment of suitable components that may be included within the controller 2802 is illustrated. As depicted, the controller 2802 includes one or more processor(s) 2804 and associated memory device(s) 2803 configured to perform a variety of computer implemented functions (e.g., performing the methods, steps, calculations and the like and storing relevant data as disclosed herein). Additionally, in some embodiments, the controller 2802 includes a communication module 2805 to facilitate communications between the controller 2802 and the various components of the system 2000.

As used herein, the term “processor” refers not only to integrated circuits referred to in the art as being included in a computer, but also refers to a controller, a microcontroller, a microcomputer, a programmable logic controller (PLC), an application specific integrated circuit, and other programmable circuits. Additionally, the memory device(s) 2803 may generally comprise memory element(s) including, but not limited to, computer readable medium (e.g., random access memory (RAM)), computer readable nonvolatile medium (e.g., a flash memory), a floppy disk, a compact disc read only memory (CD ROM), a magneto optical disk (MOD), a digital versatile disc (DVD) and/or other suitable memory elements. Such memory device(s) 2803 may generally be configured to store suitable computer readable instructions that, when implemented by the processor(s) 2804, configure the controller 2802 to perform various functions.

In some embodiments, the controller 2802 includes a recording module 2806. The recording module 2806 may be configured to record at least a delivery time and dosage volume for each dose of the medicament delivered. The recording module 1806 may, thus, facilitate the generation of an automated treatment record of the response to the acute medical condition with the system 2000. In some embodiments, recording module 2806 may be an independent module of the controller 2802. However, in some embodiments the recording module 2806 may be included within the memory device(s) 2803.

The communication module 2805 may include a sensor interface module 2807 (e.g., one or more analog to digital converters) to permit signals transmitted from one or more sensors (e.g., the longitudinal position sensors 2830, the sensing element 2810, and/or the load cell assembly 2840) to be converted into signals that can be understood and processed by the processors 2804. The sensors may be communicatively coupled to the communication module 2805 using any suitable means. For example, the sensors may be coupled to the communication module 2805 via a wired connection and/or via a wireless connection, such as by using any suitable wireless communications protocol known in the art.

The communication module 2805 can also include a power management module 2808 that may be operably coupled to the power supply 2801. In some embodiments in which the power supply 2801 is configured as a battery, the power management module 2808 can affect the power drawn from the power supply 2801 and/or provide occasion of the remaining power level. Additionally, the communication module 2805 can include a motor control module 2809 that is operably coupled to the drive assembly 2700. The motor control module 2809 may generate the command signals that affect a rotational state of each of the motors of the drive assembly 2700 in order to modify a longitudinal position of the drive members.

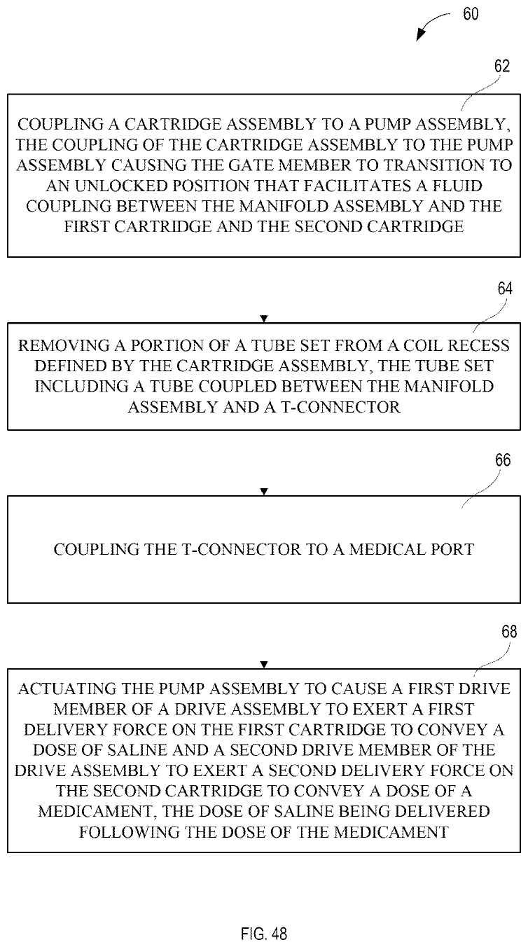

FIG. 48 is a flow chart of a method 64 administering a medication to a patient experiencing an acute medical condition. The method 60 may, in an embodiment, be performed via the Automated Medication Administration System (AMAS) 2000 and Automated Medication Administration System (AMAS) 3000 as described above with reference to FIGS. 1-47. However, it should be appreciated that in various embodiments, aspects of the method 60 may be accomplished via additional embodiments of the Automated Medication Administration System (AMAS) or components thereof, as described herein. Accordingly, the method 60 may be implemented on any suitable device as described herein. Those of ordinary skill in the art, using the disclosures provided herein, will understand that various steps of the method 60 or any of the other methods disclosed herein may be adapted, modified, rearranged, performed simultaneously, or modified in various ways without deviating from the scope of the present disclosure.

As shown at (62), the method 60 includes coupling a cartridge assembly to a pump assembly. The cartridge assembly has a gate member that maintains a separation between a manifold assembly and a first container and a second container when the gate member is in a locked position. The coupling of the cartridge assembly to the pump assembly causes the gate member to transition to an unlocked position that facilitates a fluid coupling between the manifold assembly and the first container and the second container. As shown at (64), the method 60 includes removing a portion of a tube set from a coil recess defined by the cartridge assembly. The tube set includes a tube coupled between the manifold assembly and a T-connector. Additionally, as shown at (66), the method 60 includes coupling the T-connector to a medical port. Further, as shown at (68), the method 60 includes actuating the pump assembly to cause a first drive member of a drive assembly to exert a first delivery force on the first container to convey a dose of saline and a second drive member of the drive assembly to exert a second delivery force on the second container to convey a dose of a medicament. The dose of saline being delivered following the dose of the medicament.

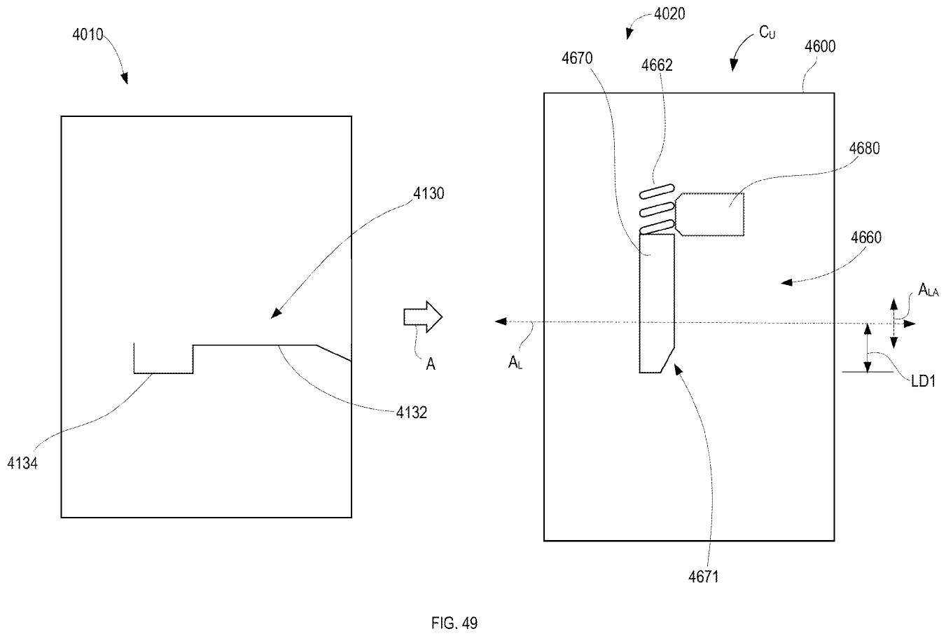

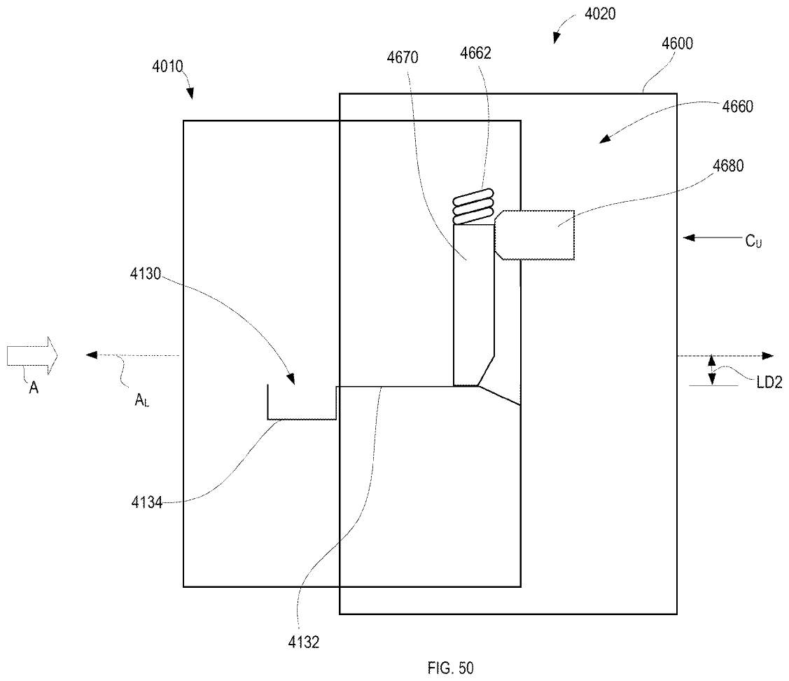

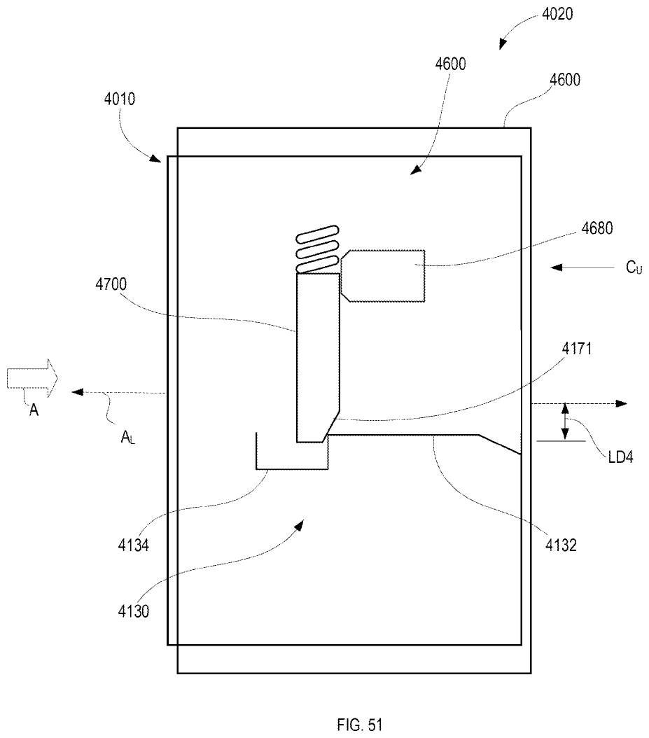

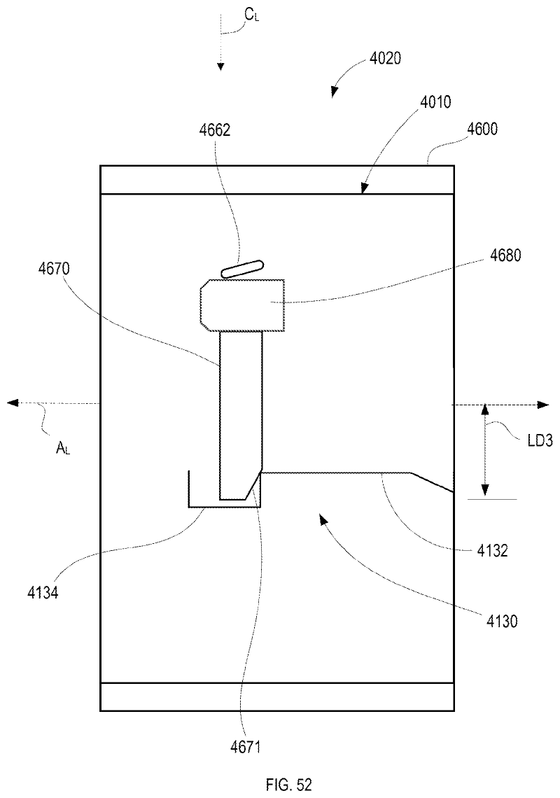

To facilitate accurate and repeatable actuation and delivery of medicament, the pump assemblies described herein are configured to receive and maintain the cartridges and cartridge assemblies described herein at a specified longitudinal location relative to the pump housing (and/or certain components of the pump assembly therein). Accordingly, in some embodiments, any of the pump assemblies described herein can include a latch member configured to engage the cartridges in cartridge assemblies to secure the cartridge at the specified longitudinal location and preclude removal from the coupling interface of the pump assembly. For example, FIGS. 49-53 are schematic illustrations of a pump assembly 4020 with a latch assembly 4660 according to an embodiment, in a first configuration (FIG. 49), a second configuration (FIG. 50), a third configuration (FIG. 52), and a fourth configuration (FIG. 51), respectively. The latch assembly 4660 can be configured to selectively couple a cartridge 4010 to the pump assembly 4020. Accordingly, the latch assembly 4460 can be configured to secure the cartridge 4010 within the pump assembly 4020 and to move the cartridge 4010 to a designated longitudinal position relative to the pump assembly 4020. Once established at the designated longitudinal position, the cartridge 4010 can be maintained in a fixed position relative to the pump assembly 4020 via the latch assembly 4660. By fixing the cartridge 4010 relative to the pump assembly 4020, the latch assembly can facilitate the dispensing of an accurate dose of a medicament from the cartridge 4010 by the pump assembly 4020.

As depicted in FIGS. 49-53, the latch assembly includes a latch arm 4670, an energy storage device 4662, and a lock member 4680. The latch arm 4670 is movably coupled to a housing 4600 of the pump assembly 4020. In some embodiments, the latch arm 4670 can, for example, be translatable along an axis that is orthogonal to a first axis (e.g., the longitudinal axis A_{L}) of the pump assembly 4020, such as a lateral axis ALA. In some embodiments, the latch arm 4670 can be translatable along an axis that is generally vertical on a condition that the pump assembly 4020 is in a generally horizontal orientation. In some embodiments, the movable coupling of the latch arm 4670 to the housing 4600 can be a rotational coupling such that the latch arm 4670 can pivot relative to the housing 4600.

In some embodiments, the movement of the latch arm 4670 can be in response to a motive force applied to the latch arm 4670 by the energy storage device 4662. Said another way, the energy storage device 4662 is operably coupled to the latch arm 4670. In some embodiments, the energy storage device 4662 can be operably coupled between the latch arm 4670 and the housing 4600. The energy storage device 4662 is configured to produce a movement of an engagement portion 4671 of the latch arm 4670. For example, the energy storage device 4662 can bias latch arm 4670 to position the engagement portion 4671 at a maximal lateral distance from a midline of the pump assembly 4020 (e.g., from the longitudinal axis A_{L}) on a condition that the cartridge 4010 is separated from the pump assembly 4020 as depicted in FIG. 49. The energy storage device 4662 can, for example, be a spring, an elastomeric member, a pneumatic member, or other suitable mechanical energy storage device.

The latch arm 4670 includes an engagement portion 4671. The engagement portion 4671 can be configured (e.g., shaped and/or positioned) to selectively engage a coupling member 4130 of the cartridge 4010. In some embodiments, the coupling member 4130 can include a keyway 4132 and a receiver structure 4134. The keyway 4132 can be configured to receive a meeting coupling protrusion (not shown) of the pump assembly 4020. The keyway 4132 can include a tapered opening to facilitate easy coupling of the cartridge 4010 to the pump assembly 4020. The coupling member 4130 also includes shoulders or other similar structures that are coupled within corresponding channels defined by the coupling protrusion of the pump assembly 4020 to limit movement of the cartridge 4010 relative to the pump assembly 4020 at least in any direction normal to the longitudinal axis A_{L} of the pump assembly 4020. The receiver structure 4134 can be configured to receive the engagement portion 4671 of the latch arm 4670.

In some embodiments, the lock member 4680 is movably coupled to the housing 4600 of the pump assembly 4020. The lock member 4680 is movable between a locked configuration C_{L} (FIG. 52) and an unlocked configuration C_{U} (FIGS. 49-51). The lock member 4680 limits the movement of the engagement portion 4671 when the lock member 4680 is in the locked configuration C_{L}. For example, in the locked configuration C_{L}, the lock member 4680 can preclude a lateral movement of the latch arm 4670 thereby establishing the engagement portion 4671 at a fixed location.

FIG. 49 depicts the pump assembly 4020 in a first configuration in which the cartridge 4010 is separated from the pump assembly 4020. The first configuration can, for example, correspond to a storage configuration of the cartridge 4010 and/or the pump assembly 4020. On a condition that the cartridge 4010 is separated from the pump assembly 4020, the engagement portion 4671 is at a first lateral distance LD1 relative to the longitudinal axis A_{L} (e.g., the midline) of the pump assembly 4020. In some embodiments, the first lateral distance LD1 can correspond to a maximal lateral position of the engagement portion 4671. In some embodiments, the first lateral distance LD1 can correspond to a maximal travel limit of the latch arm 4670. For example, the first lateral distance LD1 can correspond to a point of maximal extension of the latch arm 4670. The engagement portion 4671 can be positioned at the first lateral distance LD1 by the energy storage device 4662. Accordingly, the energy storage device 4662 can be in a discharged state on a condition that the engagement portion 4671 is at the first lateral distance LD1.

FIG. 50 depicts the pump assembly 4020 in a second configuration in which the cartridge 4010 is partially received by the pump assembly 4020 (e.g., received by a coupling interface (not shown) of the pump assembly 4020 via a movement of the cartridge 4010 in the direction indicated by arrow A). In the second configuration, the engagement portion 4671 of the latch arm 4670 is placed in contact with the coupling member 4130 (e.g., with the keyway 4132) of the cartridge 4010. The contact between the engagement portion 4671 and the coupling member 4130 moves the engagement portion 4671 from the first lateral distance LD1 to a second lateral distance LD2 relative to the longitudinal axis A_{L} of the pump assembly 4020. The second lateral distance LD2 is less than the first lateral distance LD1. In other words, the second lateral distance LD2 can correspond to a position that is closer to the midline of the pump assembly 4020 than the first lateral distance LD1. Therefore, in some embodiments, the movement of the engagement portion 4671, and thus the latch arm 4670, from the first lateral distance LD1 to the second lateral distance LD2 places energy storage device 4662 in a stored state. The stored state of the energy storage device 4662 can correspond to a compressed state of the energy storage device 4662.

FIG. 52 depicts the pump assembly 4020 in a third configuration in which the cartridge 4010 is fully inserted (e.g., positioned at the specified longitudinal location) within the pump assembly 4020 (e.g., within the coupling interface). In the third configuration, the lock member 4680 is in the locked configuration C_{L} and the engagement portion 4671 is at a third lateral distance LD3 relative to the longitudinal axis A_{L} of the pump assembly 4020. The third lateral distance LD3 is greater than the second lateral distance LD2. In some embodiments, the third lateral distance LD3 is less than the first lateral distance LD1. In some embodiments, the third lateral distance LD3 is both greater than the second lateral distance LD2 and less than the first lateral distance LD1. At the third lateral distance LD3 the engagement portion 4671 is in contact with the receiver structure 4134 of the cartridge 4010. In the locked configuration C_{L}, the lock member 4680 precludes or limits movement of the engagement portion 4671 from the third lateral distance LD3. Therefore, the lock member 4680, in the locked configuration C_{L}, maintains the contact between the engagement portion 4671 and the receiver structure of 4134 to fix the position of the cartridge 4010 relative to the housing 4600 of the pump assembly 4020. The engagement between the engagement portion 4671 and the receiver structure 4134 at the third lateral distance LD3, as maintained by the lock member 4680 limits or precludes movement of the cartridge 4010 relative to the pump assembly 4020 in a direction parallel to the longitudinal axis A_{L}. In this manner, the axial position of the cartridge 4010 can be fixed relative to the pump assembly 4020 during use, which enhances the accuracy of the dose delivered.

FIG. 51 depicts the pump assembly 4020 in a fourth configuration in which the cartridge 4010 is at least partially inserted within the pump assembly 4020 and the lock member 4680 is in the unlocked configuration C_{U}. In the fourth configuration, a greater portion of the cartridge 4010 can be inserted than in the second configuration depicted in FIG. 50. In other words, the fourth configuration can correspond to an optional intermediate position of the cartridge 4010 between the position of the cartridge 4010 in the second configuration and the position of the cartridge 4010 in the third configuration, such as may be encountered during the insertion of the cartridge 4010 into the pump assembly 4020. Therefore, in some embodiments, the cartridge 4010 can be fully inserted within the pump assembly 4020 but with the lock member in the unlocked configuration C_{U}. In the fourth configuration, the engagement portion 4671 is at a fourth lateral distance LD4 relative to the longitudinal axis A_{L} of the pump assembly 4020. The fourth lateral distance LD4 is less than the third lateral distance LD3.

At the fourth lateral distance LD4 the engagement portion 4671 is in contact with the receiver structure 4134 of the coupling member 4130 of the cartridge 4010. The contact between the engagement portion 4671 and the receiver structure 4134 develops a retention force. The retention force has a magnitude that is greater than the weight of the cartridge 4010. Therefore, the retention force of the fourth configuration, as depicted in FIG. 51, can function as a safety feature of the pump assembly 4020 that precludes an unintentional removal of the cartridge 4010 from the pump assembly 4020. However, in some embodiments, the retention force can be overcome via a force applied by the user of the pump assembly 4020. Accordingly, the fourth configuration can function as an intermediate hold state in which the cartridge 4010 is retained by the retention force prior to the actuation of the lock member 4680 until a verification sequence is completed. In the event an error is detected, or it is otherwise desirable to remove the cartridge 4010 held by the pump assembly 4020 in the fourth configuration, the retention force can be overcome by the force applied by the user (i.e., a manual force applied by the user) and the cartridge 4010 separated from the pump assembly 4020.

In some embodiments, positioning the cartridge 4010 at the specified longitudinal location relative to the pump housing 4600 can include moving the cartridge 4010 along the longitudinal axis A_{L} in the direction indicated by arrow A in FIGS. 49, 50, and 51. Accordingly, the pump assembly 4020 can be transitioned from the first configuration (FIG. 49) to the second configuration (FIG. 50), from the second configuration to the fourth configuration (FIG. 51), and from the fourth configuration to the third configuration (FIG. 52). In some embodiments, the transition of the engagement portion 4671 from the fourth lateral distance LD4 of the fourth configuration to the third lateral distance LD3 of the third configuration corresponds to a longitudinal movement of the cartridge 4010 in a proximal direction within the coupling interface of the pump assembly 4020. For example, the transition of the lock member 4680 from the unlock configuration C_{U} to the lock configuration C_{L} can produce the movement of the engagement portion 4671 from the fourth lateral distance that the third lateral distance LD3. As the engagement portion 4671 can, as depicted in FIG. 52, have an angular face, the engagement portion 4671 can, thus, act as a wedge upon the receiver structure 4134 to produce the longitudinal movement of the cartridge 4010.

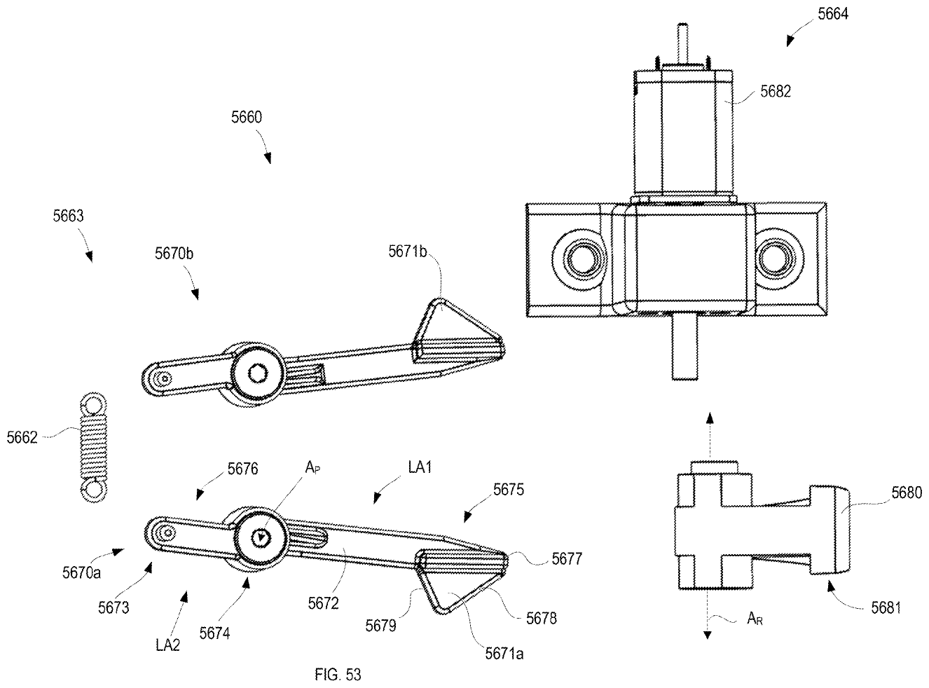

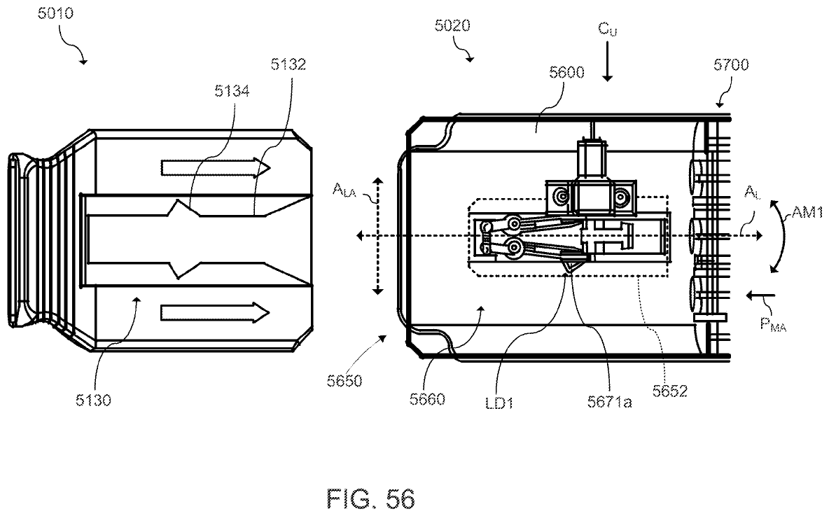

As depicted in FIGS. 53-59 in some embodiments, a latch assembly 5660 can be used to selectively couple any cartridge or cartridge assembly described herein to any pump assembly described herein. For example, as depicted in FIGS. 56-59, the latch assembly 5660 can be used to selectively couple the cartridge 5010 to a pump assembly 5020. The use of the latch assembly 5660 can facilitate accurate and repeatable actuation and delivery of medicament by the pump assembly 5020 by maintaining the cartridge 5010 at a specified longitudinal location within the pump assembly 5020. In some embodiments, the latch assembly 5660 can be configured to move the cartridge 5010 from a first position into the specified longitudinal location (e.g., to seat the cartridge 5010) via the actuation of a lock member 5680.

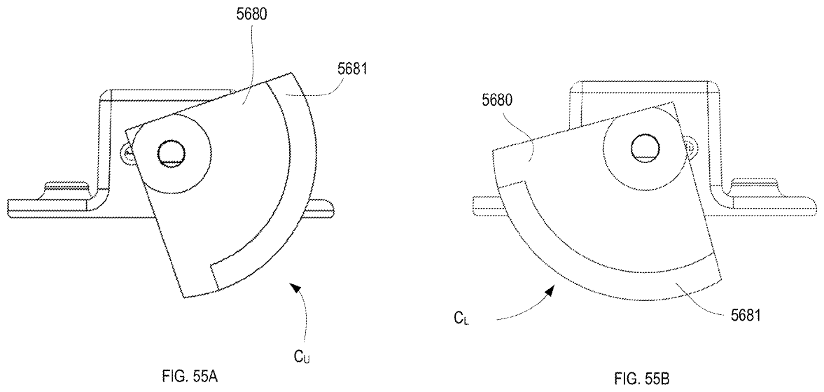

As depicted in FIG. 53, the latch assembly 5660 includes at least a first latch arm 5670a. The first latch arm 5670a is movably coupled to a housing 5600 (FIG. 56) of the pump assembly 5020 (FIG. 56). The first latch arm 5670a includes a first engagement portion 5671a that is configured (e.g., positioned and/or shaped) to selectively engage the coupling member 5130 (FIG. 56) of the cartridge 5010 (FIG. 56). The latch assembly 5660 also includes an energy storage device 5662 that is operably coupled to the first latch arm 5670a. The energy storage device 5662 is configured to produce a first movement of the first engagement portion 5671a (e.g., a lateral movement of the first engagement portion 5671a). Additionally, the latch assembly 5660 includes a lock member 5680. The lock member 5680 is movably coupled to the housing 5600 of the pump assembly 5020. The lock member 5680 is movable between a locked configuration C_{L} (FIG. 55B) and an unlocked configuration C_{U} (FIG. 55A). In the locked configuration C_{L}, the lock member 5680 engages a portion of the first latch arm 5670a. This engagement limits and/or precludes a second movement of the first engagement portion 5671a. In other words, the first engagement portion 5671a can be maintained in contact with the coupling member 5130 when the lock member 5680 is in the locked configuration C_{L}.

In some embodiments, the first latch arm 5670a includes a support structure 5672 to which the first engagement portion 5671a is coupled. For example, as depicted in FIG. 53, the first engagement portion 5671a can be coupled to a first end portion 5675 of the support structure 5672. The support structure 5672 also defines a receiving portion 5673. The receiving portion 5673 is coupled to the energy storage device 5662 and configured to receive a motive force from the energy storage device 5662. In other words, the energy storage device 5662 can apply the motive force to the receiving portion 5673 to produce the movement of the first engagement portion 5671a. The energy storage device 5662 can, for example, be a spring, an elastomeric member, a pneumatic member, or other suitable mechanical energy storage device.

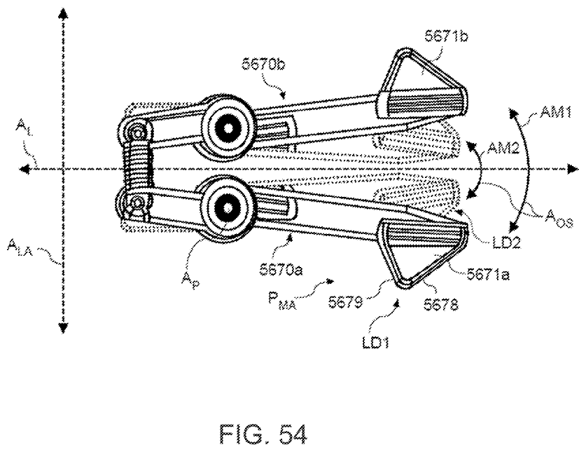

Additionally, the support structure 5672 includes and/or defines a coupling portion 5674. The first latch arm 5670a is movably coupled to the housing of the pump assembly via the coupling portion 5674. For example, in some embodiments, the coupling portion 5674 is an orifice or through hole defined by the support structure 5672 through which a fastener can be inserted to movably couple the first latch arm 5670a to the housing of the pump assembly. In some embodiments, the coupling portion 5674 defines a pivot axis Ap about which the first latch arm 5670a can rotate relative to the housing of the pump assembly, such as in response to the motive force applied by the energy storage device 5662. The pivot axis Ap can, for example, be normal to a plane defined by the longitudinal axis A_{L} and the lateral axis ALA (FIG. 54) of the pump assembly. In other words, the pivot axis Ap can form a yaw axis about which the first latch arm 5670a is configured to yaw at least in response to the motive force applied by the energy storage device 5662.

As depicted in FIG. 53, in some embodiments, the receiving portion 5673 is coupled to the support structure 5672 at a second end portion 5676. In such embodiments, the coupling portion 5674 is positioned along a length of the support structure 5672 between the first engagement portion 5671a and the receiving portion 5673. Accordingly, the support structure 5672 can define a first lever arm LA1 extending between the coupling portion 5674 and the first end portion 5675. The support structure can also define a second lever arm LA2 extending between the coupling portion 5674 and the second end portion 5676. As depicted, the length of the first lever arm LA1 is greater than the length of the second lever arm LA2. However, in some embodiments, the coupling portion 5674 can be positioned at the second end portion 5676 such that the receiving portion 5673 is positioned along the length of the support structure 5672 between the first engagement portion 5671a and the coupling portion 5674 to form a third-class lever.

In some embodiments, the support structure 5672 includes a stop protrusion 5677 extending from the support structure 5672. The stop protrusion 5677 is positioned to contact the housing 5600 of the pump assembly 5020 to establish a maximum lateral position PMA of the first engagement portion 5671a (FIG. 56). The contact between the stop protrusion 5677 and the housing 5600 can, thus, limit the movement (e.g., the lateral movement) of the first engagement portion 5671a in response to the motive force applied to the support structure 5672 by the energy storage device 5662.

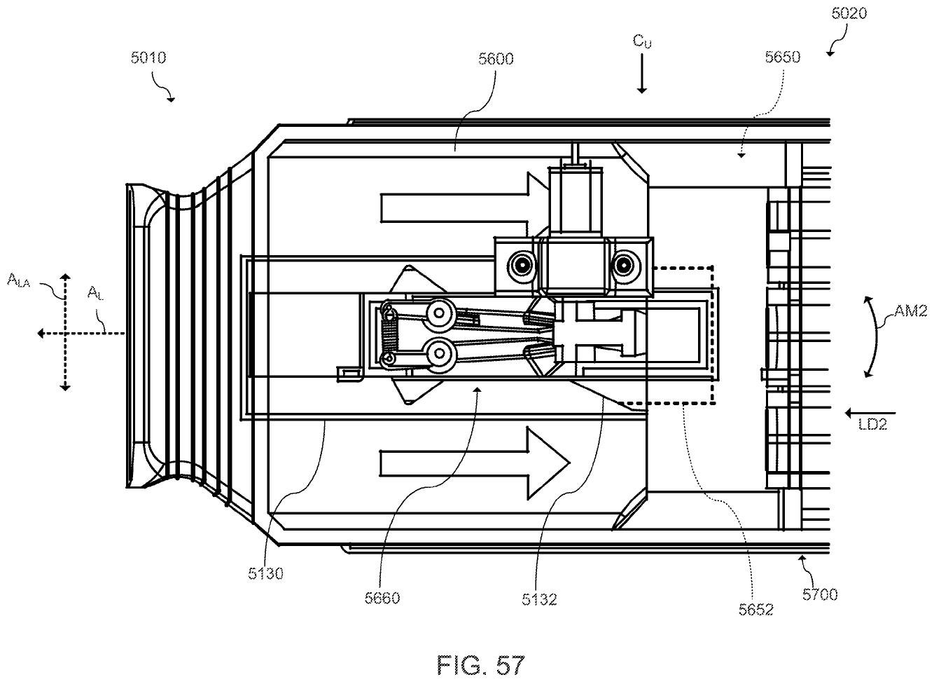

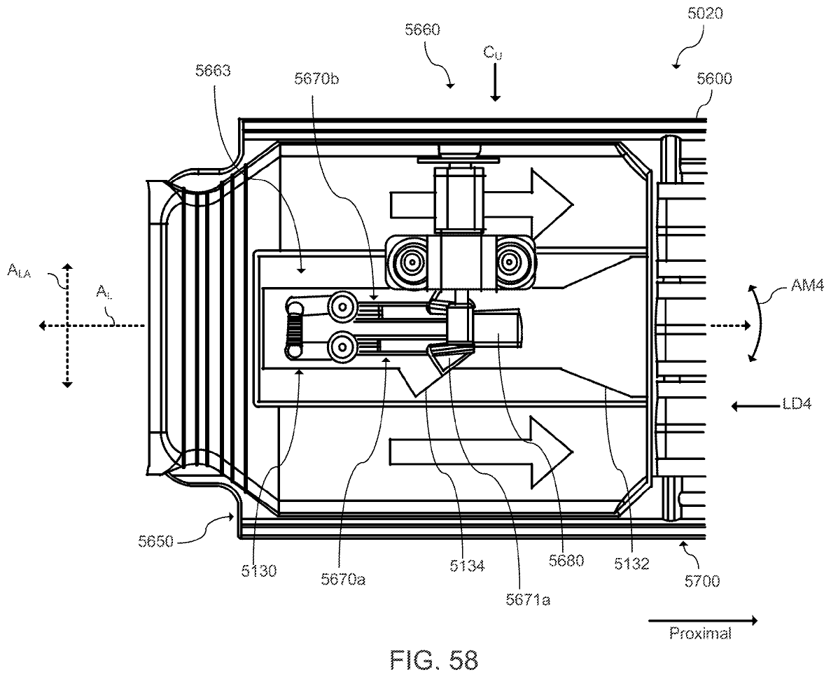

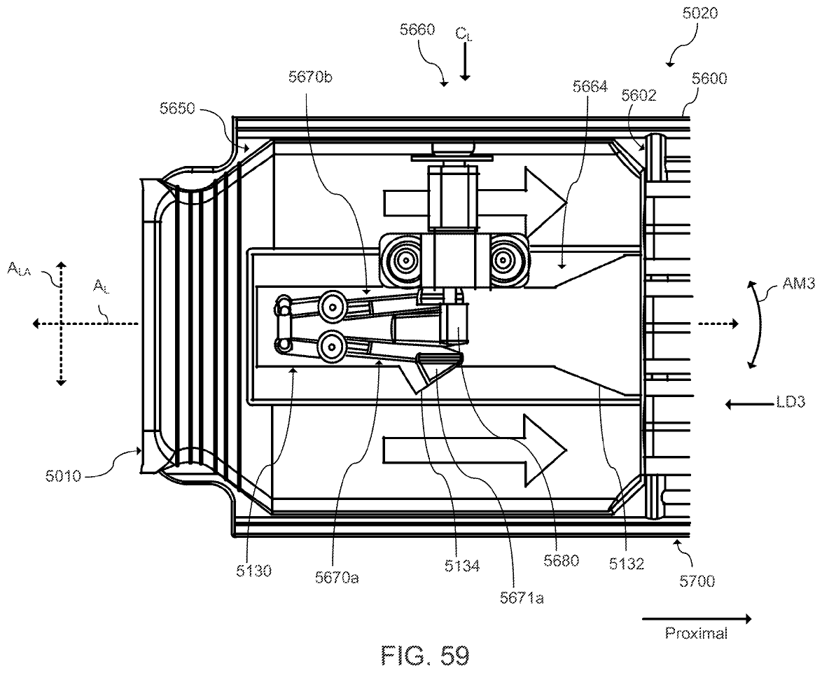

As depicted in FIG. 53, in some embodiments, the first engagement portion 5671a includes a first planar surface 5678 and a second planar surface 5679. The first planar surface 5678 is positioned to engage a corresponding surface of the coupling member 5130 (e.g., an angular feature of a receiver structure 5134) of the cartridge 5010 on a condition that the cartridge 5010 is inserted within the pump assembly 5020 as depicted in FIGS. 58 and 59. The second planar surface 5679 is positioned to engage a surface (e.g., a keyway 5132) of the cartridge 5010 to move the first engagement portion 5671a toward a midline of the pump assembly 5020 during insertion of the cartridge 5010 as depicted in FIG. 57. Accordingly, a normal vector of the first planar surface 5678 intersects with a normal vector of the second planar surface 5679. Although the surfaces 5678 and 5679 are described as being planar, in other embodiments, the first surface 5678 and/or the second surface 5679 can be slightly curved or have a non-planar portion. For example, in some embodiments, the first surface 5678 and/or the second surface 5679 can include a crowned portion.

As depicted in FIGS. 53, 55A, and 55B, in some embodiments, the lock member 5680 is a helical wedge. For example, the lock member 5680 can include at least one curved ramp 5681 on an otherwise substantially planar surface. The movement of the lock member 5680 between the unlocked configuration C_{U} depicted in FIG. 55A and the locked configuration C_{L} depicted in FIG. 55B includes a rotation of the helical wedge. In some embodiments, the helical wedge rotates about an axis of rotation A_{R} that is parallel to the lateral axis ALA (FIG. 56) of the pump assembly. In such embodiments, a design point of contact between the helical wedge and the support structure 5672 can have a first lateral distance from a midline of the pump assembly on a condition that the lock member 5680 is in the unlocked configuration C_{U} and a second, greater lateral distance from the midline on a condition that the lock member 5680 is in the locked configuration C_{L}. In some embodiments, the helical wedge rotates about an axis of rotation A_{R} that is parallel to the longitudinal axis A_{L} (FIG. 56) of the pump assembly. In still further embodiments, the helical wedge rotates about an axis of rotation A_{R} that is orthogonal to both the longitudinal axis A_{L} and the lateral axis AL_{A} of the pump assembly.

To move the lock member 5680, the latch assembly 5660 can include a motor 5682 that is operably coupled to the lock member 5680. The motor 5682 can be configured to move (e.g., rotate) the lock member 5680 between the locked configuration C_{L} and the unlocked configuration C_{U}. The lock member 5680 is separated from the cartridge 5010 when the lock member 5680 is in the locked configuration C_{L}, the unlocked configuration C_{U}, and a transition therebetween.

As depicted in FIGS. 56 and 57, in some embodiments, the first engagement portion 5671a is positioned at least partially between the housing 5600 and a portion of a coupling protrusion 5652 of the pump assembly 5020 in a manner substantially similar to that depicted in FIG. 6 with reference to the positioning of the latch assembly 2660 and the coupling protrusion 2652. Said another way, first latch arm 5670a can be positioned at least partially within the coupling interface 5650 of the pump assembly 5020. In an embodiment wherein the first engagement portion 5671a is positioned at least partially between the housing 5600 and a portion of coupling protrusion 5652, the energy storage device 5662 can be coupled between the first latch arm 5670a and the housing 5600. In such embodiments, the energy storage device 5662 can be configured to react off of the coupling with the housing 5600 to move the first engagement portion 5671a as described herein. However, in additional embodiments as described more fully below, the energy storage device 5662 can be configured to react between the first latch arm 5670a and a second latch arm 5670b.

FIG. 56 depicts the pump assembly 5020 in a first configuration in which the cartridge 5010 is separated from the pump assembly 5020. The first configuration can, for example, correspond to a storage configuration of the cartridge 5010 and/or the pump assembly 5020. On a condition that the cartridge 5010 is separated from the pump assembly 5020, the first engagement portion 5671a is at a first lateral distance LD1 relative to the longitudinal axis A_{L} (e.g., the midline) of the pump assembly 5020 as depicted in FIGS. 54 and 56. In some embodiments, the first lateral distance LD1 can correspond to a maximal lateral position P_{MA} of the first engagement portion 5671a. In some embodiments, the first lateral distance LD1 can correspond to a maximal travel limit of the first latch arm 5670a. For example, the first lateral distance LD1 can correspond to a point of maximal extension of the first latch arm 5670a. The maximal travel limit of the first latch arm 5670a can be established by contact between the housing 5600 and the stop protrusion 5677. The first engagement portion 5671a can be positioned at the first lateral distance LD1 by the energy storage device 5662. Accordingly, the energy storage device 5662 can be in a discharged state on a condition that the first engagement portion 5671a is at the first lateral distance LD1.

FIG. 57 depicts the pump assembly 5020 in a second configuration in which the cartridge 5010 is partially received by the pump assembly 5020 (e.g., received by the coupling interface 5650 of the pump assembly 5020). In the second configuration, the first engagement portion 5671a of the first latch arm 5670a is placed in contact with the coupling member 5130 of the cartridge 5010. More specifically, the second planar surface 5679 is placed in contact with the keyway 5132. The contact between the first engagement portion 5671a and the coupling member 5130 moves the first engagement portion 5671a from the first lateral distance LD1 to a second lateral distance LD2 relative to the longitudinal axis A_{L} of the pump assembly 5020. The second lateral distance LD2 is less than the first lateral distance LD1. In other words, the second lateral distance LD2 can correspond to a position that is closer to the midline of the pump assembly 5020 than the first lateral distance LD1. In some embodiments, the second lateral distance LD2 can correspond to a minimal lateral distance from the midline. Positioning the first engagement portion 5671a at the second lateral distance LD2 can correspondingly position the receiving portion 5673 of the support structure 5672 further from the midline than is the case when the first engagement portion 5671a is at the first lateral distance LD1, as is depicted by the dashed latch arm in FIG. 54. Therefore, in some embodiments, the movement of the first engagement portion 5671a, and thus the first latch arm 5670a, from the first lateral distance LD1 to the second lateral distance LD2 places energy storage device 5662 in a stored state. The stored state of the energy storage device 5662 can correspond to a compressed state of the energy storage device 5662.

FIG. 59 depicts the pump assembly 5020 in a third configuration in which the cartridge 5010 is fully inserted (e.g., positioned at the specified longitudinal location) within the pump assembly 5020 (e.g., within the coupling interface 5650). The third configuration can, for example, correspond to a ready and/or actuated configuration of the pump assembly 5020. In some embodiments, in the third configuration, a proximal portion of the cartridge 5010 can be placed in contact with (e.g., abutting) a portion of the wall 5602 of the housing 5600 forming a proximal boundary of the coupling interface 5650. In the third configuration, the lock member 5680 is in the locked configuration C_{L} and the first engagement portion 5671a is at a third lateral distance LD3 relative to the longitudinal axis A_{L} of the pump assembly 5020. The third lateral distance LD3 is greater than the second lateral distance LD2. In some embodiments, the third lateral distance LD3 is less than the first lateral distance LD1. In some embodiments, the third lateral distance LD3 is both greater than the second lateral distance LD2 and less than the first lateral distance LD1. At the third lateral distance LD3 the first engagement portion 5671a is in contact with the receiver structure 5134 of the cartridge 5010. In the locked configuration C_{L}, the lock member 5680 precludes or limits movement of the first engagement portion 5671a from the third lateral distance LD3. Therefore, the lock member 5680, in the locked configuration C_{L}, maintains the contact between the first engagement portion 5671 a and the receiver structure of 5134 to fix the position of the cartridge 5010 relative to the housing 5600 of the pump assembly 5020. The engagement between the first engagement portion 5671a and the receiver structure 5134 at the third lateral distance LD3, as maintained by the lock member 5680 limits or precludes movement of the cartridge 5010 relative to the pump assembly 5020 in a direction parallel to the longitudinal axis A_{L}. In this manner, the axial position of the cartridge 5010 can be fixed relative to the pump assembly 5020 during use, which enhances the accuracy of the dose delivered.

FIG. 58 depicts the pump assembly 5020 in a fourth configuration in which the cartridge 5010 is at least partially inserted within the pump assembly 5020 and the lock member 5680 is in the unlocked configuration C_{U}. In the fourth configuration, a greater portion of the cartridge 5010 can be inserted than in the second configuration depicted in FIG. 57. In other words, the fourth configuration can correspond to an intermediate position of the cartridge 5010 between the position of the cartridge 5010 in the second configuration and the position of the cartridge 5010 in the third configuration. This intermediate position can be encountered during the insertion of the cartridge 5010 into the pump assembly 5020. Therefore, in some embodiments, the cartridge 5010 can be fully inserted within the pump assembly 5020 but with the lock member in the unlocked configuration C_{U}. In the fourth configuration, the first engagement portion 5671a is at a fourth lateral distance LD4 relative to the longitudinal axis A_{L} of the pump assembly 5020. The fourth lateral distance LD4 is less than the third lateral distance LD3.

At the fourth lateral distance LD4 the first engagement portion 5671a is in contact with the receiver structure 5134 of the coupling member 5130 of the cartridge 5010. More specifically, the first planar surface 5678 is in contact with a corresponding annular surface of the receiver structure 5134 of the coupling member 5130. The contact between the first engagement portion 5671a and the receiver structure 5134 develops a retention force. The retention force has a magnitude that is greater than the weight of the cartridge 5010. Therefore, the retention force of the fourth configuration, as depicted in FIG. 58, can function as a safety feature of the pump assembly 5020 that precludes an unintentional removal of the cartridge 5010 from the pump assembly 5020. However, in some embodiments, the retention force can be overcome via a force applied by the user of the pump assembly 5020. Accordingly, the fourth configuration can function as an intermediate hold state in which the cartridge 5010 is retained by the retention force prior to the actuation of the lock member 5680 until a verification sequence is completed. In the event an error is detected, or it is otherwise desirable to remove the cartridge 5010 held by the pump assembly 5020 in the fourth configuration, the retention force can be overcome by the force applied by the user and the cartridge 5010 separated from the pump assembly 5020.

In some embodiments, positioning the cartridge 5010 at the specified longitudinal location relative to the pump housing 5600 can include moving the cartridge 5010 along the longitudinal axis AL. Accordingly, the pump assembly 5020 can be transitioned from the first configuration (FIG. 56) to the second configuration (FIG. 57), from the second configuration to the fourth configuration (FIG. 58), and from the fourth configuration to the third configuration (FIG. 59). In some embodiments, the transition of the first engagement portion 5671a from the fourth lateral distance LD4 of the fourth configuration to the third lateral distance LD3 of the third configuration corresponds to a longitudinal movement of the cartridge 5010 in a proximal direction within the coupling interface 5650 of the pump assembly 5020.

For example, the transition of the lock member 5680 from the unlock configuration C_{U} to the lock configuration C_{L} can produce the movement of the first engagement portion 5671a from the fourth lateral distance to the third lateral distance LD3. In some embodiments, the rotation of the helical wedge generates a force that acts against the support structure 5672 to produce a lateral movement of the first engagement portion 5671a away from the midline of the pump assembly 5020 in the direction of the third lateral distance LD3. Accordingly, the first planar surface 5678 (e.g., an angular face) of the first engagement portion 5671a can act as a wedge upon the receiver structure 5134 to produce the longitudinal movement of the cartridge 5010 in response to the lateral movement of the first engagement portion 5671a, which is, in turn, a response to the force developed by the rotation of the helical wedge. It should be appreciated that in an embodiment in which the cartridge 5010 is at the designated longitudinal position, as depicted in FIG. 59, additional longitudinal movement of the cartridge 5010 in the proximal direction is limited by the housing 5600 of the pump assembly 5020. Therefore, the rotation of the helical wedge results in the development of a fixing force between the first engagement portion 5671a and the receiver structure 5134 to maintain the cartridge 5010 at the designated longitudinal position.

As depicted in FIGS. 53-59, in some embodiments the first engagement portion 5671a is configured to move in a first lateral direction relative to the midline of the pump assembly 5020. In such embodiments, the latch assembly 5660 includes a second latch arm 5670b. In a manner substantially similar to the first latch arm 5670a, the second latch arm 5670b is movably coupled to the housing 5600 of the pump assembly 5020. In some embodiments, the first latch arm 5670a and the second latch arm 5670b are coupled to the housing 5600 on opposing sides of the longitudinal axis A_{L} and equidistant therefrom. The second latch arm 5670b can, for example, be a mirror copy of the first latch arm 5670a including the same structures, features, and/or components as described herein with reference to the first latch arm 5670a. Accordingly, the second latch arm 5670b includes a second engagement portion 5671b. The second engagement portion 5671b is configured to selectively engage the coupling member 5130 of the cartridge 5010.

In some embodiments, the second latch arm 5670b is movable in a second lateral direction via the energy storage device 5662. In some embodiments, the energy storage device 5662 is coupled between the first latch arm 5670a and the second latch arm 5670b. Therefore, the second lateral direction is opposite the first lateral direction of the first latch arm 5670a. Additionally, a displacement of the first engagement portion 5671a in the first lateral direction is substantially equal to a displacement of the second engagement portion 5671b in the second lateral direction. On a condition that the lock member 5680 is in the locked configuration C_{L}, the lock member 5680 can limit and/or preclude movement of both the first engagement portion 5671a and the second engagement portion 5671b away from the coupling member 5130. In other words, the lock member 5680 in the locked configuration C_{L} can preclude lateral movement of the first engagement portion 5671a and the second engagement portion 5671b toward the longitudinal axis AL (e.g., the midline) of the pump assembly 5020.

In some embodiments, the energy storage device 5662, the lock member 5680 in the locked configuration C_{L}, and/or contact between the first and second engagement portions 5671a, 5671b and the coupling member 5130 establish an angular separation AoS (FIG. 54) between the first engagement portion 5671a and the second engagement portion 5671b. As depicted in FIG. 56, on a condition in which the cartridge 5010 is separated from the pump assembly 5020 (i.e., the pump assembly 5020 is in the first configuration), the angular separation AoS between the first engagement portion 5671a and the second engagement portion 5671b has a first angular magnitude AM1.

During the insertion of the cartridge 5010 into the coupling interface 5650 of the pump assembly 5020, the first engagement portion 5671a and the second engagement portion 5671b are brought into contact with the coupling member 5130 (e.g., with the keyway 5132). The longitudinal movement in the proximal direction of the cartridge 5010 during insertion results in lateral movements of both the first engagement portion 5671a and the second engagement portion 5671b toward the longitudinal axis A_{L} of the pump assembly 5020 as depicted in FIG. 57 (e.g., the establishment of the pump assembly 5020 in the second configuration). This lateral movement toward the midline of the pump assembly 5020 establishes the angular separation AoS at a second angular magnitude AM2. The second angular magnitude AM2 is less than the first angular magnitude AM1.

As depicted in FIG. 59, on a condition in which the lock member 5680 is in the locked configuration C_{L}, (i.e., the pump assembly 5020 is in the third configuration) the angular separation AoS between the first engagement portion 5671a and the second engagement portion 5671b has a third angular magnitude AM3. The third angular magnitude AM3 is greater than the second angular magnitude AM2. In other words, the lateral distance between the first engagement portion 5671 a and the second engagement portion 5671b is centered about the midline of the pump assembly 5020 and is greater in the third configuration depicted by FIG. 59 than the second configuration depicted by FIG. 57. However, to ensure that the force developed between the first and second engagement portions 5671a, 5671b and the receiver structure 5134 is not limited by contact between the stop protrusion(s) 5677 and the housing 5600, the first angular magnitude AM1 is the maximal angular magnitude. Accordingly, the third angular magnitude AM3 is less than the first angular magnitude AM1. Therefore, the force developed on the first and second engagement portions 5671a, 5671b by the rotation of the lock member 5680, which wedges the first engagement portion 5671 a away from the second engagement portion 5671b, is transferred to the and the receiver structure 5134 rather than the housing 5600.

FIG. 58 depicts the pump assembly 5020 in the fourth configuration as previously described. On a condition in which the first engagement portion 5671a and the second engagement portion 5671b are in contact with a receiver structure 5134 of the coupling member 5130 of the cartridge 5010 and the lock member 5680 is in the unlocked configuration C_{U}, the angular separation AoS has a fourth angular magnitude AM4. The fourth angular magnitude AM4 is less than the third angular magnitude AM3. In other words, the lateral separation between the first engagement portion 5671a and the second engagement portion 5671b indicated by the fourth angular magnitude AM4 when the pump assembly is in the fourth configuration is less than the lateral separation indicated by the third angular magnitude AM3 and the first angular magnitude AM1. However, the fourth angular magnitude AM4 can be greater than the second angular magnitude AM2.

On a condition that the angular separation AoS has the fourth angular magnitude AM4, the first engagement portion 5671a and second engagement portion 5671b are in contact with the receiver structure 5134 of the coupling member 5130 of the cartridge 5010 to develop a retention force. The retention force has a magnitude that is greater than the weight of the cartridge 5010. Therefore, the retention force of the fourth configuration, as depicted in FIG. 58, can function as a safety feature of the pump assembly 5020 that precludes an unintentional removal of the cartridge 5010 from the pump assembly 5020. However, in some embodiments, the retention force can be overcome via a force applied by the user of the pump assembly 5022 the cartridge 5010. Accordingly, the fourth configuration can function as an intermediate hold state in which the cartridge 5010 is retained by the retention force prior to the actuation of the lock member 5680 until a verification sequence is completed. In the event an error is detected, or it is otherwise desirable to remove the cartridge 5010 held by the pump assembly 5020 in the fourth configuration, the retention force can be overcome by the force applied by the user and the cartridge 5010 separated from the pump assembly 5020.

In some embodiments, an increase in the angular separation AoS from the fourth angular magnitude AM4 to the third angular magnitude AM3 corresponds to a longitudinal movement of the cartridge 5010 within the coupling interface 5650 in a proximal direction. For example, insofar as the lock member 5680 is configured as a helical wedge, the rotation of the lock member 5680 toward the locked configuration C_{L} results in an increase in the thickness of the lock member 5680 at a point of contact between the lock member 5680 and the first and second latch arms 5670a, 5670b. In embodiments wherein the lock member 5680 is positioned along the longitudinal axis A_{L} of the pump assembly 5020, the increase in the thickness of the lock member 5680 resulting from the rotation of the helical wedge increases the separation between the first engagement portion 5671a and the second engagement portion 5671b. As the first engagement portion 5671a and the second engagement portion 5671b can, as depicted in FIG. 58, have an angular face, the first and second engagement portions 5671a, 5671b can, thus, act as wedges that act upon the corresponding surfaces of the receiver structure 5134 to produce the longitudinal movement of the cartridge 5010 until the specified longitudinal position is obtained. At which point, the same interaction resulting from separation between the first engagement portion 5671a and the second engagement portion 5671b in response to the rotation of the lock member 5680 serves to fix the cartridge 5010 at the specified longitudinal position as depicted in FIG. 59. For example, a proximal end portion of the cartridge 5010 can abut a portion of the wall 5602 of the housing 5600 at the specified longitudinal position. In other words, the portion of the wall 5602 can limit the longitudinal movement of the cartridge 5010 in the proximal direction.

As depicted in FIGS. 53-59, in some embodiments, the pump assembly 5020 is a pump assembly for delivering a medicament. The pump assembly 5020 can include the pump housing 5600. The pump housing can include the coupling interface 5650 configured to receive the cartridge 5010 and/or a cartridge assembly, such as described herein. The pump assembly 5020 can also include a drive assembly 5700 positioned within the pump housing 5600 and configured to cause the delivery of the dose of medicament from the cartridge 5010 positioned at the specified longitudinal location relative to the pump housing 5600. Additionally, the pump assembly 5020 can, as described herein, include the latch assembly 5660. Latch assembly 5660 is positioned to selectively secure the cartridge 5010 within the coupling interface 5650. The latch assembly 5660 includes a passive lock mechanism 5663 and an active lock mechanism 5664. The passive lock mechanism 5663 is configured to retain the cartridge assembly 5010 within the coupling interface 5650 while still permitting the removal of the cartridge assembly 5010 in response to an external force applied to the cartridge assembly 5010. In other words, the passive lock mechanism 5663 is configured to retain the cartridge assembly 5010 on a condition that the pump assembly 5020 is in the fourth configuration as depicted in FIG. 58. The active lock mechanism 5664 is configured to secure the cartridge assembly 5010 at the specified longitudinal location and preclude removal of the cartridge assembly 5010 from the coupling interface 5650. In other words, the active lock mechanism 5664 is configured to secure the cartridge assembly 5010 on a condition that the pump assembly 5020 is in the third configuration as depicted in FIG. 59. Accordingly, the active lock mechanism 5664 can include the lock member 5680 that is movable between the locked configuration C_{L} depicted in FIG. 59 and the unlocked configuration C_{U} depicted in FIG. 58.

In some embodiments, the passive lock mechanism 5663 can include the energy storage device 5662 operably coupled to at least the first latch arm 5670a as described herein. The energy storage device 5662 is configured to establish the first engagement portion 5671a in contact with the receiver structure 5134 of the cartridge assembly 5010 to develop the retention force. As previously described, the retention force has a magnitude that is greater than the weight of the cartridge assembly 5010. On a condition that the lock member 5680 of the active lock mechanism 5664 is in the unlocked configuration C_{U} the cartridge assembly 5010 is retained within the coupling interface 5650 via the passive lock mechanism 5663. However, on a condition that the lock member 5680 is in the locked configuration C_{L}, the lock member 5680 maintains the first engagement portion 5671a in contact with the receiver structure 5134 so that the cartridge assembly 5010 is fixed at the specified longitudinal location.

Although various embodiments have been described as having particular features and/or combinations of components, other embodiments are possible having a combination of any features and/or components from any of embodiments as discussed above.

1. A pump assembly for delivering a medicament, the pump assembly comprising:
a pump housing having a wall, the wall including an inner face defining an internal volume, the wall including a coupling interface positioned to engage a coupling member of a cartridge assembly having a first container and a second container, the first container containing saline and the second container containing a medicament, the coupling interface including a coupling protrusion positioned to engage an actuation portion of a gate member of the cartridge assembly, the engagement of the actuation portion by the coupling protrusion transitions the gate member from a locked position to an unlocked position; and
a drive assembly positioned within the internal volume, the drive assembly including a first drive member and a second drive member, the first drive member operably coupled to the first container and configured to produce a first delivery force to convey a dose of saline from the first container when the gate member is in the unlocked position, the second drive member operably coupled to the second container and configured to produce a second delivery force to convey a dose of the medicament from the second container when the gate member is in the unlocked position, the first drive member being actuatable independently of the second drive member.

2. The pump assembly of claim 1, wherein:
the first drive member is a first piston configured to exert the first delivery force on a first elastomeric member of the first container to convey the dose of saline; and
the second drive member is a second piston configured to exert a second delivery force on a second elastomeric member of the second container to convey a dose of medicament.

3. The pump assembly of claim 2, wherein:
the cartridge assembly includes a manifold assembly in fluid communications with a tube set;
the tube set is configured to be coupled to a medical port of a patient;
the second delivery force exerted on the second elastomeric member is configured to convey the dose of the medicament from the second container to the patient via the manifold assembly and the tube set; and
the first delivery force exerted on the first elastomeric member is configured to flush the dose of the medicament from the manifold assembly, the tube set, and the medical port via the dose of saline from the first container.

4. The pump assembly of claim 3, wherein a delivery volume of the cartridge assembly is defined by a fluid volume of the manifold assembly, a fluid volume of the tube set, and the fluid volume of the medical port, the pump assembly further comprising:
a controller operably coupled to the drive assembly, the controller comprising at least one processor configured to perform a plurality of operations, the plurality of operations comprising controlling the first delivery force such that a magnitude of the dose of saline is greater than or equal to 1.0 and less than or equal to 1.1 times the delivery volume.

5. The pump assembly of claim 1, wherein:
the drive assembly includes a first actuation member surrounding the first drive member and a second actuation member surrounding the second drive member,
the first actuation member having at least one engagement lug positioned to be received by one of a plurality of actuation slots of the cartridge assembly, the at least one engagement lug of the first actuation member is positioned to exert a first actuation force on a first container body of the first container in response to the pump assembly being actuated, the first actuation force causing the first container to be placed in fluid communication with a manifold assembly of the cartridge assembly,
the second actuation member having at least one engagement lug positioned to be received by one of the plurality of actuation slots of the cartridge assembly, the at least one engagement lug of the second actuation member is positioned to exert a second actuation force on a second container body of the second container in response to the pump assembly being actuated the second actuation force causing the second container to be placed in fluid communication with the manifold assembly.

6. The pump assembly of claim 5, wherein:
the first actuation force causes the first container to move into contact with a first puncturer of the cartridge assembly; and
the second actuation force causes the second container to move into contact with a second puncturer of the cartridge assembly.

7. The pump assembly of claim 5, further comprising:
a first energy storage device surrounding the first drive member, the first energy storage device being positioned between the wall and the first actuation member to generate the first actuation force in a distal direction when actuated; and
a second energy storage device surrounding the second drive member, the second energy storage device being positioned between the wall and the second actuation member to generate the second actuation force in the distal direction when actuated.

8. The pump assembly of claim 7, wherein:
the first energy storage device and the second energy storage device are positioned at least partially within corresponding recesses defined by the wall.

9. The pump assembly of claim 7, wherein:
the first drive member includes a first retention portion positioned to engage the first actuation member when the first drive member is in a ready state to establish a separation distance between the at least one engagement lug of the first actuation member and the first container body; and
the second drive member includes a second retention portion positioned to engage the second actuation member when the second drive member is in the ready state to establish the separation distance between the at least one engagement lug of the second actuation member and the second container body.

10. The pump assembly of claim 9, wherein:
the first drive member is configured to disengage from the first actuation member and is positioned to exert the first delivery force on a first elastomeric member of the first container when in an actuated state;
the at least one engagement lug of the first actuation member is configured to exert the first actuation force on the first container body when in the actuated state;
the second drive member is configured to disengage from the second actuation member and is positioned to exert the second delivery force on a second elastomeric member of the second container when in the actuated state; and
the at least one engagement lug of the second actuation member is configured to exert the second actuation force on the second container body when in the actuated state.

11. The pump assembly of claim 10, wherein:
the first energy storage device and the second energy storage device are positioned to bias the first container body and the second container body against the manifold assembly when in the actuated state.

12. The pump assembly of claim 1, wherein:
the cartridge assembly includes a manifold assembly configured to move in a proximal direction and into fluid communication with the first container and the second container in response to the gate member being moved to the unlocked position.

13. The pump assembly of claim 1, further comprising:
a cap member sized to selectively occlude the coupling interface when the pump assembly is in a stored state.

14. The pump assembly of claim 1, wherein the coupling interface includes:
a latch assembly positioned to selectively couple the cartridge assembly to the pump assembly.

15. The pump assembly of claim 1, wherein the drive assembly includes:
a drive assembly chassis defining a plurality of guide slots extending parallel to a longitudinal axis of the pump assembly;
a first motor and a first lead screw supported by the drive assembly chassis and operably coupled to the first drive member, the first drive member includes a first piston guide block configured to be received by one of the plurality of guide slots; and
a second motor and a second lead screw supported by the drive assembly chassis and operably coupled to the second drive member, the second drive member includes a second piston guide block configured to be received by one of the plurality of guide slots defined by the drive assembly chassis wherein:
each of the first motor and the second motor define a motor axis that is parallel to the longitudinal axis,
each of the first lead screw and the second lead screw define a lead-screw axis that is parallel to the longitudinal axis and is displaced from the motor axis, and
each guide slot of the plurality of guide slots is displaced from each motor axis and each lead screw axis.

16. The pump assembly of claim 15, further comprising:
a plurality of longitudinal-position sensors coupled to the drive assembly chassis, the plurality of longitudinal-position sensors being positioned to monitor a longitudinal position of the first drive member and the second drive member.

17. The pump assembly of claim 15, further comprising:
a load cell assembly positioned to monitor an axial load of the first lead screw and the second lead screw in a proximal direction.

18. The pump assembly of claim 1, further comprising:
an output assembly configured to deliver a plurality of outputs to an operator of the pump assembly;
an input assembly configured to receive a plurality of inputs from the operator; and
a controller operably coupled to the output assembly, the input assembly, and the drive assembly, the controller comprising at least one processor configured to perform a plurality of operations, the plurality of operations comprising:
determining a cartridge assembly classification of the cartridge assembly based on an input from a sensing element,
implementing a treatment protocol based on the cartridge assembly classification, the treatment protocol including a medicament dosage and delivery schedule,
generating at least one output of the plurality of outputs that corresponds to a dose-delivery prompt in accordance with the treatment protocol,
receiving a dose-delivery input from the operator,
in response to the dose-delivery input, delivering a first command signal to the drive assembly, the first command signal causing the second drive member to exert the second delivery force on a second elastomeric member of the second container to convey the dose of the medicament,
following the conveying of the dose of medicament, delivering a second command signal to the drive assembly, the second command signal causing the first drive member to exert the first delivery force on a first elastomeric member of the first container to convey the dose of saline, and
delivering additional doses of the medicament in accordance with the treatment protocol followed by additional doses of saline.

19. The pump assembly of claim 18, wherein the plurality of operations include:
recording a delivery time and dosage volume for each dose of the medicament delivered; and
generating an automated treatment record.

20. The pump assembly of claim 18, wherein the plurality of operations include:
receiving a cartridge-status input from the sensing element;
determining a deviation of the cartridge-status input from a cartridge-status design value; and
delivering an error signal to the output assembly in response to the deviation between the cartridge-status input and the cartridge-status design value.

21. The pump assembly of claim 18, wherein the plurality of operations include:
delivering a third command signal to the drive assembly to implement a priming of the cartridge assembly.

22. The pump assembly of claim 1, wherein the pump housing has a ratio of a lateral length to a longitudinal length that is between 1:2.40 and 1:2.60.

23. A method for administering a medication to a patient experiencing an acute medical condition, the method comprising:
coupling a cartridge assembly to a pump assembly, the cartridge assembly having a gate member that maintains a separation between a manifold assembly and a first container and a second container when the gate member is in a locked position, the coupling of the cartridge assembly to the pump assembly causing the gate member to transition to an unlocked position that facilitates a fluid coupling between the manifold assembly and the first container and the second container;
removing a portion of a tube set from a coil recess defined by the cartridge assembly, the tube set including a tube coupled between the manifold assembly and a T-connector;
coupling the T-connector to a medical port; and
actuating the pump assembly to cause a first drive member of a drive assembly to exert a first delivery force on the first container to convey a dose of saline and a second drive member of the drive assembly to exert a second delivery force on the second container to convey a dose of a medicament, the dose of saline being delivered following the dose of the medicament.

24-52. (canceled)
